SEQUENCE LISTING (119 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2-3 - skipped (2 entries)

SEQ ID NO: 4 - protein, 13 aa, synthetic construct
  SITE 13: note = C-terminus can be linked to N-terminus of SEQ ID NO: 5 via a Trioxatridecan-succinamic acid linker to form Peptide 11
NNQGNMQREPNQA

SEQ ID NO: 5 - protein, 15 aa, Homo sapiens
  MOD_RES 6: note = PHOSPHORYLATION
  MOD_RES 10: note = PHOSPHORYLATION
  SITE 1: note = N-terminus can be linked to C-terminus of SEQ ID NO: 4 or 6 via a Trioxatridecan-succinamic acid linker to form Peptide 11 or 23, respectively
GNNSYSGSNSGAAIG

SEQ ID NO: 6 - protein, 31 aa, synthetic construct
  SITE 31: note = C-terminus linked to N-terminus of SEQ ID NO: 5 via a Trioxatridecan-succinamic acid linker to form Peptide 23
SKQSQDEPLRSRKVFVTEDMTEDELREFFSQ

SEQ ID NO: 7 - protein, 28 aa, synthetic construct
  SITE 28: note = C-terminus linked to N-terminus of SEQ ID NO: 8 or 9 via a Trioxatridecan-succinamic acid linker to form Peptide 28 or 29, respectively
TEDMTEDELREFFSQNNQGNMQREPNQA

SEQ ID NO: 8 - protein, 14 aa, Homo sapiens
  MOD_RES 8: note = Phosphorylated serine
  MOD_RES 9: note = Phosphorylated serine
  SITE 1: note = N-terminus can be linked to SEQ ID NO: 7 C-terminus to form Peptide 28 via a Trioxatridecan-succinamic acid linker
GFNGGFGSSMDSKS

SEQ ID NO: 9 - protein, 13 aa, Homo sapiens
  MOD_RES 9: note = Phosphorylated serine
  MOD_RES 10: note = Phosphorylated serine
  SITE 1: note = N-terminus can be linked to SEQ ID NO: 3 C-terminus to form Peptide 29 via a Trioxatridecan-succinamic acid linker
FGSSMDSKSSGWG

SEQ ID NO: 10 - protein, 116 aa, synthetic construct
EVQLVESGGGLVHPKGSLQLSCAASGFTFNTYTMHWVRQAPGKGLEWVARIRSKRSNYATYYADSVKDRFTISRDDSQSMLYLQMNNLKTEDTAIYYCVRGTGSYWGQGTSVTVSS

SEQ ID NO: 11 - protein, 5 aa, synthetic construct
TYTMH

SEQ ID NO: 12 - protein, 19 aa, synthetic construct
RIRSKRSNYATYYADSVKD

SEQ ID NO: 13 - protein, 5 aa, synthetic construct
GTGSY

SEQ ID NO: 14 - protein, 112 aa, synthetic construct
DVVMTQTPLTLSVTIGQPASISCRSSQSLLDRDGETYLNWLLQRPGQSPKRLIYLVSKLDSGVPDRFTGSGSGTDFTLKISRVEAEDLGVYYCWEGTHLPFAFGSGTKLEIK

SEQ ID NO: 15 - protein, 16 aa, synthetic construct
RSSQSLLDRDGETYLN

SEQ ID NO: 16 - protein, 7 aa, synthetic construct
LVSKLDS

SEQ ID NO: 17 - protein, 9 aa, synthetic construct
WEGTHLPFA

SEQ ID NO: 18 - DNA, 348 bp, synthetic construct
  source 1..348: mol_type = other DNA
gaggtgcagcttgttgagtctggtggaggattggtgcaccctaaaggatcattgcaactctcatgtgccgcctctggtttcaccttcaatacctataccatgcactgggtccgccaggctccaggaaagggtttggaatgggttgctcgcataagaagtaaaaggagtaattatgcaacatattatgccgattcagtgaaagacagattcaccatctccagagatgattcacaaagcatgctctatctgcaaatgaacaacctgaaaactgaggacacagccatatattactgtgtgagaggcacgggaagttactggggtcaaggaacctcagtcaccgtctcctca

SEQ ID NO: 19 - DNA, 336 bp, synthetic construct
  source 1..336: mol_type = other DNA
gatgttgtgatgacccagactccactcactttgtcggttaccattggacagccagcctccatctcttgcaggtcaagtcagagcctcttagatcgtgatggagagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctgggaaggtacacatcttccattcgcgttcggctcggggacaaagttggaaataaaa

SEQ ID NO: 20 - protein, 117 aa, synthetic construct
EIQLQQSGTVMARPGASVKMSCKTSGYIFTNYWMHWVKQRPGQGLDWIGTIYPGNSDTDYNQNFKGKAKLTAVTSASTAYMELSSLTNEDSAVYFCIRGGWGGFPYWGQGTLVTVSA

SEQ ID NO: 21 - protein, 5 aa, synthetic construct
NYWMH

SEQ ID NO: 22 - protein, 17 aa, synthetic construct
TIYPGNSDTDYNQNFKG

SEQ ID NO: 23 - protein, 8 aa, synthetic construct
GGWGGFPY

SEQ ID NO: 24 - protein, 112 aa, synthetic construct
DVVMTQIPLSLPVSLGDQASISCRSSQRLVHSNGNTYLHWYLQKPGQSPKFLIHKVSNRFSGVPDRFSGSGSGTDFTLKIRRVEAEDLGVYFCCQSTHVPYTFGGGTKLEIK

SEQ ID NO: 25 - protein, 16 aa, synthetic construct
RSSQRLVHSNGNTYLH

SEQ ID NO: 26 - protein, 7 aa, synthetic construct
KVSNRFS

SEQ ID NO: 27 - protein, 9 aa, synthetic construct
CQSTHVPYT

SEQ ID NO: 28 - DNA, 351 bp, synthetic construct
  source 1..351: mol_type = other DNA
gaaattcagctccagcagtctgggactgtgatggcaaggcctggggcttcagtgaagatgtcctgcaagacttctggctacatatttaccaactactggatgcactgggtaaaacagaggcctggacagggtctggactggatagggactatttatcctggaaatagtgatactgactacaaccagaactttaagggcaaggccaaactgactgcagtcacatccgccagcactgcctacatggagctcagcagcctgacaaatgaggactctgcggtctatttctgtataagagggggatggggagggtttccttactggggccaagggactctggtcactgtctctgca

SEQ ID NO: 29 - DNA, 336 bp, synthetic construct
  source 1..336: mol_type = other DNA
gatgttgtgatgacccaaattccactctccctgcctgtcagtcttggagatcaagcctccatctcttgcagatctagtcagagacttgtacacagtaatggaaacacctatttacattggtacctgcagaagccaggccagtctccaaagttcctgatccacaaagtttccaaccgattttctggggtcccagacaggttcagtggcagtggatcagggacagatttcacactcaagatcagaagagtggaggctgaggatctgggagtatatttctgctgtcaaagtacacatgttccgtacacgttcggaggggggaccaagctggaaataaaa

SEQ ID NO: 30 - protein, 114 aa, synthetic construct
QVQLQQPGTELVKPGASVKLSCKASGYTFTRYWMHWMKQRPGQGLEWIGNINPSDGGTNYNEKFKNKASLTVDKSSSTAYMQLSRLTSEDSAVYYCARRGSGYWGQGTTLTVSS

SEQ ID NO: 31 - protein, 5 aa, synthetic construct
RYWMH

SEQ ID NO: 32 - protein, 17 aa, synthetic construct
NINPSDGGTNYNEKFKN

SEQ ID NO: 33 - protein, 5 aa, synthetic construct
RGSGY

SEQ ID NO: 34 - protein, 112 aa, synthetic construct
DVVMTQTPLILSVTIGQPASISCKSGQSLLDSDGWTYLNWMFQRPGQSPKRLIYLVSKLDSGVPDRFTGSGSGTDFTLKISRVEAEDLGVYHCWQGTHLPYTFGGGTTLEIK

SEQ ID NO: 35 - protein, 16 aa, synthetic construct
KSGQSLLDSDGWTYLN

SEQ ID NO: 36 - skipped

SEQ ID NO: 37 - protein, 9 aa, synthetic construct
WQGTHLPYT

SEQ ID NO: 38 - DNA, 342 bp, synthetic construct
  source 1..342: mol_type = other DNA
caggtccaattacagcagcctgggactgaactggtgaagcctggggcttcagtgaaactgtcctgcaaggcttctggctacaccttcaccaggtactggatgcactggatgaaacagaggccaggacaaggccttgagtggattggaaatattaatcctagcgatggtggtaccaactacaatgagaagttcaagaataaggcctcactgactgtagacaaatcctccagtacagcctacatgcagctcagcaggctgacatctgaggactcggcggtctattattgtgcaagacgcggctcgggctactggggccaaggcaccactctcacagtctcctca

SEQ ID NO: 39 - DNA, 336 bp, synthetic construct
  source 1..336: mol_type = other DNA
gatgttgtgatgacccagactccactcattttgtcggttaccattggacaaccagcctccatctcttgcaagtcaggtcagagcctcttagatagtgatggatggacatatcttaattggatgttccagcggccaggccagtctccaaagcgcctaatctatctggtgtctaaactggactctggagtccctgacaggttcactggcagtggatcagggacagatttcacactgaagatcagcagagtggaggctgaggatttgggagtttatcattgctggcaaggtacacatcttccgtacacgttcggaggggggaccacgctggaaataaaa

SEQ ID NO: 40 - protein, 114 aa, synthetic construct
QVQLQQPGTELVKPGASVKLSCKASGYTFTSYWMHWVKQRPGQGLEWIGNINPINSDTNYNEKFKTKATLTVDKSSSTAYMQLSSLTSEDSAVYYCARRGSGYWGQGTTLTVSS

SEQ ID NO: 41 - protein, 5 aa, synthetic construct
SYWMH

SEQ ID NO: 42 - protein, 17 aa, synthetic construct
NINPINSDTNYNEKFKT

SEQ ID NO: 43 - skipped

SEQ ID NO: 44 - protein, 112 aa, synthetic construct
DVVMTQTPLTLSVTIGQPAFISCKSSQSLLDSNGKTYLNWMLQRPGQSPKRLIYLVAKLDSGVPDRLTGSGSGTDYTLKISRVEAEDLGVYYCWQGTHIPYTFGGGTKLEIK

SEQ ID NO: 45 - protein, 16 aa, synthetic construct
KSSQSLLDSNGKTYLN

SEQ ID NO: 46 - protein, 7 aa, synthetic construct
LVAKLDS

SEQ ID NO: 47 - protein, 9 aa, synthetic construct
WQGTHIPYT

SEQ ID NO: 48 - DNA, 342 bp, synthetic construct
  source 1..342: mol_type = other DNA
caggtccaactgcagcagcctgggactgaactggtgaagcctggggcttcagtgaagctgtcctgcaaggcttctggctacaccttcaccagctactggatgcactgggtgaagcagaggcctggacaaggccttgagtggattggaaatattaatcctatcaatagtgatactaactacaatgagaagttcaagaccaaggccacactgactgtagacaaatcctccagcacagcctacatgcagctcagcagcctgacatctgaggactctgcggtctattattgtgcaagacgcggctcgggctactggggccaaggcaccactctcacagtctcctca

SEQ ID NO: 49 - DNA, 336 bp, synthetic construct
  source 1..336: mol_type = other DNA
gatgttgtgatgacccagactccactcactttgtcggttaccattggacaaccagccttcatctcttgcaagtcaagtcagagcctcttagatagtaatggaaagacatatctgaattggatgttacagaggccaggccagtctccaaagcgcctaatctatctggtggctaaactggactctggagtccctgacaggctcactggcagcggatcagggacagattacacactgaaaatcagcagagtggaggctgaggatttgggagtttattattgctggcaaggtacacatattccgtacacgttcggaggggggaccaagctggaaataaaa

SEQ ID NO: 50 - protein, 113 aa, synthetic construct
EEHLVESGGGLVKPGGSLKLSCAASGFTFSDYGMQWVRQAPEKGLEWVAYISSGSSTIYYEDTVKGRFTISRDNAKNTLFLQMTSLRSEDTAMYYCVRPYDNYGQGTLVTVSA

SEQ ID NO: 51 - protein, 5 aa, synthetic construct
DYGMQ

SEQ ID NO: 52 - protein, 17 aa, synthetic construct
YISSGSSTIYYEDTVKG

SEQ ID NO: 53 - protein, 5 aa, synthetic construct
PYDNY

SEQ ID NO: 54 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDVGVYYCFQGSHVPRTFGGGTKLEIK

SEQ ID NO: 55 - protein, 16 aa, synthetic construct
RSSQSIVHSNGNTYLE

SEQ ID NO: 56 - skipped

SEQ ID NO: 57 - protein, 9 aa, synthetic construct
FQGSHVPRT

SEQ ID NO: 58 - DNA, 339 bp, synthetic construct
  source 1..339: mol_type = other DNA
gaggagcacctggtggagtctgggggaggcttagtgaagcctggagggtccctgaaactctcctgtgcagcctctggattcactttcagtgactatggaatgcagtgggttcgtcaggctccagagaaggggctggagtgggttgcatacattagtagtggcagtagtaccatctactatgaagacacagtgaagggccgattcaccatctccagagacaatgccaagaacaccctgttcctgcaaatgaccagtctgaggtctgaggacacggccatgtactactgtgtaaggccctatgataactacggccaagggactctggtcactgtctctgca

SEQ ID NO: 59 - DNA, 336 bp, synthetic construct
  source 1..336: mol_type = other DNA
gatgttttgatgacccagactccactctccctgcctgtcagtcttggagatcaagcctccatctcttgcagatctagtcagagtattgtccatagtaatggaaacacctatttagaatggtacctgcagaaacctggccagtctccaaagctcctgatctacaaagtttccaatcgattttctggggtcccagacaggttcagtggcagtggatcagggacagatttcacactgaagatcagtagagtggaggctgaggatgtgggagtttattactgctttcaaggttcacatgttcctcggacgttcgggggaggcaccaagctggaaatcaaa

SEQ ID NO: 60 - protein, 113 aa, synthetic construct
EVQLVESGGGLVKPGGSLKLSCAASGFTFSDYGMHWVRQAPEKGLEWVAYISSGSSTIYYADTVKGRFTMSRDNAKNTLFLQMTSLRSEDTAMYYCSRPYGNYGQGTLVTVSA

SEQ ID NO: 61 - protein, 5 aa, synthetic construct
DYGMH

SEQ ID NO: 62 - protein, 17 aa, synthetic construct
YISSGSSTIYYADTVKG

SEQ ID NO: 63 - protein, 5 aa, synthetic construct
PYGNY

SEQ ID NO: 64 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 65-67 - skipped (3 entries)

SEQ ID NO: 68 - DNA, 339 bp, synthetic construct
  source 1..339: mol_type = other DNA
gaggtgcagctggtggagtctgggggaggcttagtgaagcctggagggtccctgaaactctcctgtgcagcctctggattcactttcagtgactatggaatgcactgggttcgtcaggctccagagaaggggctggagtgggttgcatacattagtagtggcagtagtaccatctactatgcagacacagtgaagggccgattcaccatgtccagagacaatgccaagaacaccctgttcctgcaaatgaccagtctgaggtctgaggacacggccatgtattactgttcaaggccctatggaaactacggccaagggactctggtcactgtctctgca

SEQ ID NO: 69 - DNA, 336 bp, synthetic construct
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 70-73 - skipped (4 entries)

SEQ ID NO: 74 - protein, 111 aa, synthetic construct
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPWTFGGGTKLEIK

SEQ ID NO: 75 - protein, 15 aa, synthetic construct
RASKSVSTSGYSYMH

SEQ ID NO: 76 - protein, 7 aa, synthetic construct
LASNLES

SEQ ID NO: 77 - protein, 9 aa, synthetic construct
QHSRELPWT

SEQ ID NO: 78 - skipped

SEQ ID NO: 79 - DNA, 333 bp, synthetic construct
  source 1..333: mol_type = other DNA
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccaagctggaaatcaaa

SEQ ID NO: 80 - protein, 113 aa, synthetic construct
QVQLKQSGPELVKPGASVKLSCKASGYTFTTYDINWVKQRPGQGPEWIGWIYPRVGNTKYNEKFKDKATLTVDTSSSTAYMELHSLTSEDSAVYFCASALTYWGQGTLVTVSA

SEQ ID NO: 81 - protein, 5 aa, synthetic construct
TYDIN

SEQ ID NO: 82 - protein, 17 aa, synthetic construct
WIYPRVGNTKYNEKFKD

SEQ ID NO: 83 - protein, 4 aa, synthetic construct
ALTY

SEQ ID NO: 84 - protein, 112 aa, synthetic construct
DVLMTQTPLTLSVTIGQPASISCKSSQSLLDSDGKTYLNWLFQRPGQSPKRLIYLVSKLDSGVPDRFTGSGSGTDFTLKISRVEAEDLGVYYCFQGSHVPLTFGAGTKLELK

SEQ ID NO: 85 - protein, 16 aa, synthetic construct
KSSQSLLDSDGKTYLN

SEQ ID NO: 86 - skipped

SEQ ID NO: 87 - protein, 9 aa, synthetic construct
FQGSHVPLT

SEQ ID NO: 88 - DNA, 339 bp, synthetic construct
  source 1..339: mol_type = other DNA
caggtgcagctgaagcagtctggacctgagctggtgaagcctggggcttcagtgaagttgtcctgcaaggcttctggctacaccttcacaacctacgatataaactgggtgaagcagaggcctggacagggacctgagtggattggatggatttatcctagagttggtaatactaagtacaatgagaagttcaaggacaaggccacattgactgtagacacatcctccagcacagcgtacatggagctccacagcctgacatctgaggactctgcggtctatttctgtgcaagcgcgttgacctactggggccaagggactctggtcactgtctctgca

SEQ ID NO: 89 - DNA, 336 bp, synthetic construct
  source 1..336: mol_type = other DNA
gatgttttgatgacccaaactccactcactttgtcggttaccattggacaaccagcctccatctcttgcaagtcaagtcagagcctcttagatagtgatggaaagacatatttgaattggttgtttcagaggccaggccagtctccaaagcgcctaatctatctggtgtctaaactggactctggagtccctgacaggttcactggcagtggatcagggacagatttcacactgaaaatcagcagagtggaggctgaggatttgggagtttattactgctttcaaggttcacatgttcctctcacgttcggtgctgggaccaagctggagctgaaa

SEQ ID NO: 90 - protein, 120 aa, synthetic construct
EVHLVESGGDLVMPGGSLKLSCAASGFTFSNYGMSWVRQTPDKRLEWVATISSGGKYINYLDSLKGRFTISRDNAKNTLYLQMSSLKSEDTAMYYCAKDYGSGWAWFAYWGQGTLVTVSA

SEQ ID NO: 91 - protein, 5 aa, synthetic construct
NYGMS

SEQ ID NO: 92 - protein, 17 aa, synthetic construct
TISSGGKYINYLDSLKG

SEQ ID NO: 93 - protein, 11 aa, synthetic construct
DYGSGWAWFAY

SEQ ID NO: 94 - protein, 115 aa, synthetic construct
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVL

SEQ ID NO: 95 - protein, 12 aa, synthetic construct
TLSSQHSTYTIE

SEQ ID NO: 96 - protein, 7 aa, synthetic construct
GSHSTGD

SEQ ID NO: 97 - protein, 13 aa, synthetic construct
GVGDTIKEQFVYV

SEQ ID NO: 98 - DNA, 360 bp, synthetic construct
  source 1..360: mol_type = other DNA
gaggtacatctggtggagtctgggggagacttagtgatgcctggagggtccctgaagctctcctgtgcagcctctggattcactttcagtaactatggcatgtcttgggttcgccagactccagacaagaggctggagtgggtcgcaaccattagtagtggtggtaaatatatcaactacttagacagtttgaaggggcgattcaccatctccagagacaatgccaagaacaccctatacctgcaaatgagcagtctgaagtctgaggatacagccatgtattactgtgcaaaagactacggtagtggctgggcctggtttgcttactggggccaagggactctggtcactgtctctgca

SEQ ID NO: 99 - DNA, 345 bp, synthetic construct
  source 1..345: mol_type = other DNA
caacttgtgctcactcagtcatcttcagcctctttctccctgggagcctcagcaaaactcacgtgcaccttgagtagtcagcacagtacgtacaccattgaatggtatcagcaacagccactcaagcctcctaagtatgtgatggagcttaagaaagatggaagccacagcacaggtgatgggattcctgatcgcttctctggatccagctctggtgctgatcgctaccttagcatttccaacatccagcctgaagatgaagcaatatacatctgtggtgtgggtgatacaattaaggaacaatttgtgtatgttttcggcggtggaaccaaggtcactgtccta

SEQ ID NO: 100 - protein, 121 aa, synthetic construct
QVQLKESGPGLVAPSQSLSITCTVSGFSLNRNGVQWVRQPPGKGLEWLGVIWPGGSTNCNSALMSRLSISKDNSKSQVFLKMNSLHTDDTGIYYCARVGGNYVWDYNNYAWGQGTLVTVSA

SEQ ID NO: 101 - protein, 5 aa, synthetic construct
RNGVQ

SEQ ID NO: 102 - protein, 16 aa, synthetic construct
VIWPGGSTNCNSALMS

SEQ ID NO: 103 - protein, 13 aa, synthetic construct
VGGNYVWDYNNYA

SEQ ID NO: 104 - protein, 112 aa, synthetic construct
DVLMTQTPLSLPVSLGDQASISCRSSQNIVHSIGNTYLEWYLQKPGQSPKLLIYKVSNRFSGVPDRFSGSGSGTDFTLKISRVEAEDLGVYYCFQGSHVPYTFGGGTKLEIR

SEQ ID NO: 105 - protein, 16 aa, synthetic construct
RSSQNIVHSIGNTYLE

SEQ ID NO: 106 - skipped

SEQ ID NO: 107 - protein, 9 aa, synthetic construct
FQGSHVPYT

SEQ ID NO: 108 - DNA, 363 bp, synthetic construct
  source 1..363: mol_type = other DNA
caggtgcagctgaaggagtcaggacctggcctggtggcgccctcacagagcctgtccatcacttgcactgtctctgggttttcgttaaacagaaatggtgtacagtgggttcgccagcctccaggaaagggtctggagtggctgggagtaatatggcctggcggaagcacaaattgtaattcggctctcatgtccagactgagcatcagcaaagacaactccaagagtcaagttttcttaaaaatgaacagtctgcacactgatgacacaggcatatattactgtgccagagtagggggtaactacgtgtgggactataataactacgcctggggccaagggactctggtcactgtctctgca

SEQ ID NO: 109 - DNA, 336 bp, synthetic construct
  source 1..336: mol_type = other DNA
gatgttttgatgacccaaactccactctccctgcctgtcagtcttggagatcaggcctccatctcttgcagatctagtcagaacattgtacatagtattggaaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctagaaataaga

SEQ ID NO: 110 - protein, 116 aa, synthetic construct
DVQLVESGGGLVQPGGSRKLSCEASGFTFSSIGMHWVRQAPEKGLEWVAYITSGSSTIYYADTVKGRFTISRDNPKNTLFLQMTSLRSEDTAIYYCAGSGPGTDYWGQGTTLTVSS

SEQ ID NO: 111 - protein, 5 aa, synthetic construct
SIGMH

SEQ ID NO: 112 - protein, 17 aa, synthetic construct
YITSGSSTIYYADTVKG

SEQ ID NO: 113 - protein, 7 aa, synthetic construct
SGPGTDY

SEQ ID NO: 114 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELK

SEQ ID NO: 115 - protein, 11 aa, synthetic construct
KASQDINSYLS

SEQ ID NO: 116 - protein, 7 aa, synthetic construct
RANRLVD

SEQ ID NO: 117 - protein, 9 aa, synthetic construct
LQYDEFPLT

SEQ ID NO: 118 - DNA, 348 bp, synthetic construct
  source 1..348: mol_type = other DNA
gatgtgcagctggtggagtctgggggaggcttagtgcagcctggagggtcccggaaactctcctgtgaagcctctggattcactttcagtagcattggaatgcactgggttcgtcaggctccagagaaggggctggagtgggtcgcatatattactagtggcagtagtaccatctactatgcagacacagtgaagggccgattcaccatctccagagacaatcccaagaacaccctgttcctgcaaatgaccagtctaaggtctgaggacacggccatctattactgtgcaggatctggacctgggactgactactggggccaaggcaccactctcacagtctcctca

SEQ ID NO: 119 - DNA, 321 bp, synthetic construct
  source 1..321: mol_type = other DNA
gacatcaagatgacccagtctccatcttccatgtatgcgtctctaggagagagagtcactatcacttgcaaggcgagtcaggacattaatagctatttaagctggttccagcagaaaccagggaaatctcctaagaccctgatctatcgtgcaaacagattggttgatggggtcccatcaaggttcagtggcagtggatctgggcaagattattctctcaccatcagcagcctggagtatgaagatatgggaatttattattgtctacagtatgatgagtttcctctcacgttcggtgctgggaccaagctggagctgaaa

(19) United States
(12) Patent Application Publication
(10) Pub. No.: US 2026/0043816 A1
(43) Pub. Date: Feb. 12, 2026

(54) Anti-TDP-43 Binding Molecules

(71) Applicant: AC Immune SA, Lausanne (CH)

(72) Inventors: Oskar Adolfsson, Lausanne (CH); Ruth Luthi-Carter, Lausanne (CH); Tamara Seredenin, Lausanne (CH)

(21) Appl. No.: 18/854,434

(22) PCT Filed: Apr. 6, 2023
(86) PCT No.: PCT/EP2023/059222

§ 371 Date: Oct. 4, 2024

(30) Foreign Application Priority Data
Apr. 8, 2022 (EP) 22167341.1
Mar. 28, 2023 (EP) 23164858.5

(51) Int. Cl. G01N 33/68; C07K 16/18; G01N 33/58

(52) U.S. Cl. CPC G01N 33/6896; C07K 16/18; G01N 33/581; G01N 33/6857; C07K 2317/34; C07K 2317/565; C07K 2317/92; G01N 2440/14; G01N 2800/2821

(57) ABSTRACT

TDP-43 binding molecules specifically binding phosphorylated TDP-43 are provided, together with the nucleic acid molecules that encode the binding molecules. These binding molecules are useful in diagnostic and therapeutic applications and may be included in suitable compositions and kits. They may be used in pairing assays involving use of capture and detect antibody pairs. They may be used to monitor diseases associated with TDP-43, including for testing candidate therapeutic agents.

### FIELD OF THE INVENTION

The present invention is in the field of transactive response DNA binding protein with a molecular weight of 43 kDa (TDP-43 or TARDBP). The invention relates to molecules that bind to TDP-43 and related TARDBP gene products, in particular to anti-TDP-43 antibodies or an antigen-binding fragment or a derivative thereof and uses thereof. The present invention provides means and methods to diagnose, prevent, alleviate and/or treat a disorder and/or abnormality associated with TDP-43 and its various isoforms including, but not limited to, Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Alzheimer's Disease (AD), Down Syndrome (DS), Parkinson's Disease (PD) and related disorders (including PD with Dementia (PDD), dementia with Lewy Bodies (DLB), multiple system atrophy (MSA)), limbic-predominant age-related TDP-43 encephalopathy (LATE), myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy, and diseases of both sporadic and hereditary origins, including genetic cases arising from mutations or variant-associated risk alleles of the progranulin (GRN), TARDBP, C9ORF72, valosin-containing protein (VCP), angiogenin (ANG), desmin (DES), myotilin (MYOT), TMEM106B genes. The present invention provides novel, high-affinity antibodies and antibody derivatives with unique sequences, structures and stabilities for use as tools in the diagnosis, prevention, monitoring, alleviation and/or treatment of TDP-43-related disorders and/or abnormalities. The invention also provides novel epitope-specific TDP-43-binding molecules to allow targeting and detection of previously inaccessible forms of TDP-43 in vivo or in human biological samples, either alone or in combination.

### REFERENCE TO ELECTRONIC SEQUENCE LISTING

The contents of the electronic sequence listing (BOULT-069_SEQUENCE LISTING; Size: 108,642 bytes; and Date of Creation: Oct. 4, 2024) is herein incorporated by reference in its entirety.

### BACKGROUND

### TDP-43 and its Role in Central Nervous System Proteinopathies

Disorders (proteinopathies) characterized by pathological aggregation of proteins in the central nervous system (CNS) and peripheral organs represent one of the leading causes of disability and mortality in the world. One of the proteins that undergoes pathological aggregation is the transactive response DNA binding protein with a molecular weight of 43 kDa (TARDBP or TDP-43). TDP-43 proteinopathies (arising from aggregated forms of TDP-43) include, but are not limited to, Amyotrophic Lateral Sclerosis (ALS), Frontotemporal Dementias (FTDs, including Argyrophilic grain disease), Frontotemporal Lobar Degeneration with Motor Neuron Disease FTLD-MND, Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD) dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy (Lagier-Tourenne et al., Human Molecular Genetics, 2010, Vol. 19, Review Issue 1 R46-R64; de Boer et al., Journal of Neurology, Neurosurgery and Psychiatry 2020 Vol. 92, Issue 1, 86-95, Nelson et al., Journal of Neuropathology & Experimental Neurology 2020, Vol. 75, Issue 6, June 2016, Pages 482-498). These comprise diseases of both sporadic and hereditary origins, including genetic cases arising from mutations or variant-associated risk alleles of the progranulin (GRN), TARDBP, valosin-containing protein (VCP), angiogenin (ANG), huntingtin (HTT), ataxin 3 (ATXN3), desmin (DES) or myotilin (MYOT) genes.

In 2006, TDP-43 was identified as the protein that accumulates in the vast majority of cases of frontotemporal lobar degeneration (FTLD) with tau-negative, ubiquitin-positive inclusions (then referred to as FTLD-TDP), and in most cases of amyotrophic lateral sclerosis (ALS) (Arai et al., Biochemical and Biophysical Research Communications 351 (2006) 602-611; Neumann et al., Science 314, (2006), 130-133).

Aggregated TDP-43 from patient brains shows a number of abnormal modifications, including hyperphosphorylation, ubiquitination, acetylation and C-terminal fragments through proteolytic cleavage (Arai et al., Biochemical and Biophysical Research Communications 351 (2006) 602-611; Neumann et al., Science 314, (2006), 130-133; Neumann et al., Acta Neuropathol. (2009) 117: 137-149; Hasegawa et al., (2008) Annals of Neurology Vol 64 No 1, 60-70; Cohen et al., Nat Commun. 6: 5845, 2015). Another characteristic feature of TDP-43 pathology is redistribution and accumulation of TDP-43 from nucleus to cytoplasm. The hallmark lesions of FTLD-TDP are neuronal and glial cytoplasmic inclusions (NCI and GCI, respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology are described so far by histological classification (Mackenzie and Neumann, J. Neurochem. (2016) 138 (Suppl. 1), 54-70). FTLD-TDP type A cases are characterized by abundant short dystrophic neuritis (DN) and compact oval or crescentic NCI, predominantly in layer II of the neocortex (FIG. 2f in Mackenzie et al., 2016 J. Neurochem. 138 (Suppl. 1), 54-70). Cases with this pathology usually present clinically with either behavioural-variant frontotemporal dementia (bvFTD) or nonfluent/agrammatic variants of Primary Progressive Aphasia (nfvPPA) and are associated with progranulin (GRN) mutations. Type B cases show moderate numbers of compact or granular NCI in both superficial and deep cortical layers with relatively few DN and NII (neuronal intranuclear inclusions; FIG. 2g in Mackenzie et al., 2016 J. Neurochem. 138 (Suppl. 1), 54-70). Most cases with co-appearance of FTD and ALS symptoms are found to have FTLD-TDP type B pathology. Type C cases have an abundance of long, tortuous neurites, predominantly in the superficial cortical laminae, with few or no NCI (FIG. 2j in Mackenzie et al., 2016 J. Neurochem. 138 (Suppl. 1), 54-70). This pathology is particularly found in cases presenting with semantic variant of primary progressive aphasia (svPPA). FTLD-TDP type D displays with abundant lentiform neuronal intranuclear inclusions (NII) and short DN in the neocortex with only rare NCI (FIG. 2k in Mackenzie et al., 2016 J. Neurochem. 138 (Suppl. 1), 54-70). This pattern of pathology is only found in cases with VCP in association with inclusion body myositis (IBM).

### Genetic Forms of TDP-43 Proteinopathies

Thirty-eight dominant-negative mutations in TARDBP have been identified in sporadic and familial ALS patients as well as in patients with inherited FTD mainly located in the sequence encoding the glycine rich domain (FIG. 1 in Lagier-Tourenne and Cleveland, Cell 136, 2009, 1001-1004). ALS-associated TARDBP mutations render the protein more aggregation-prone (Ticozzi et al., CNS Neurol. Disord. Drug Targets. 2010, 9(3), 285-296.) connecting TDP-43 aggregation with clinical disease manifestation.

### TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique 169 (2013) 811-819). FTD is presented by several syndromes including bvFTD which is characterized by changes in personality and behaviour; semantic dementia (SD) and progressive nonfluent aphasia (PNFA) characterized by changes in the language function; corticobasal syndrome (CBS), progressive supranuclear palsy syndrome and motor neuron disease (FTD-MND) characterized by movement dysfunction. Clinical diagnosis of these syndromes is complicated and final conclusion can only be achieved through postmortem histopathological analysis to detect aggregated protein and define affected brain regions. In terms of pathological, proteinaceous inclusions, about 45% of cases show pathological accumulation of misfolded Tau, 45% of cases have pathological TDP-43 and a smaller subgroup has aggregates of fused in sarcoma (FUS) protein and other proteins.

### TDP-43 in ALS

Amyotrophic lateral sclerosis (ALS) is a neurodegenerative disorder characterized by the premature loss of upper and lower motor neurons. The progression of ALS is marked by fatal paralysis and respiratory failure with a disease course from diagnosis to death of 1 to 5 years. In most cases of sporadic ALS, the neuropathology is characterized by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of antibodies that specifically bind to phosphorylated TDP-43 in nuclear and cytoplasmic inclusions with amino acids S379, S403, S404, S409, S410 as the major sites of phosphorylation of TDP-43 (Hasegawa et al., Ann Neurol 2008; 64: 60-70; Neumann et al., Acta Neuropathol (2009) 117: 137-149).

### TDP-43 in AD

TDP-43 pathology occurs in up to 57% of brains of patients with Alzheimer's disease (Josephs K A et al., Acta Neuropathol. 2014; 127(6): 811-824; Josephs K A et al., Acta Neuropathol. 2014; 127(3): 441-450; McAleese et al., Brain Pathol. 2017 July; 27(4): 472-479). TDP-43 aggregation is associated with patient's age and correlates with cognitive decline, memory loss and medial temporal atrophy in AD. It appears that in AD TDP-43 represents a secondary or independent pathology that shares overlapping brain distribution with amyloid beta and Tau pathologies in the medial temporal lobe. AD-associated pathologic TDP-43 follows a stereotypical pattern of progressive deposition that has been described by the so-called TDP-43 in AD (TAD) staging scheme: TDP-43 first deposits in the amygdala (stage I) followed by hippocampus, limbic, temporal, and finally the frontostriatum (stage V) (Josephs K A et al., Acta Neuropathol. 2014; 127(6): 811-824; Josephs K A et al., Acta Neuropathol. 2014; 127(3): 441-450).

### Pathologic Spreading of Aggregated TDP-43

Although ALS and FTD onset and first symptoms vary significantly between patients, the common feature of disease progression is spreading of pathology from an initial focal area to most neurons. The continuous worsening of symptoms might be explained by the progressive spread of TDP-43 pathology. TDP-43 pathology in an ALS patient's brain appears to be spreading in a four-stage process and it is believed that propagation occurs transynaptically via corticofugal axonal projections using anterograde axonal transport (Brettschneider et al., Ann Neurol. 2013 July; 74(1): 20-38.). Recent experimental evidence indicates that pathological protein propagation of amyloid-beta, tau, alpha-synuclein and TDP-43 in neural tissue occurs by shared prion-like mechanisms (Hasegawa et al., 2017), but with distinct starting points and topographical spreading patterns (Brettschneider J et al., Nature Rev. Neuroscience, 2015, 109). One facet of these shared propagation mechanisms is believed to be the cell-to-cell spreading of pathological protein aggregates. This process consists of the release of aggregates from a diseased cell, uptake by a naïve (or less-affected) cell and subsequent seeding of the pathological protein conformation by a templated conformational change in intracellular proteins. In the case of TDP-43, the sequestration of functional TDP-43 protein may be sufficiently extreme to cause a cellular deficit (comprising a loss-of-function mechanism).

Cell-to-cell spreading of TDP-43 has been studied at a molecular level in several in vitro models, where insoluble TDP-43 preparations from patient brain are able to induce intracellular aggregate formation in receptor cells (Nonaka et al., Cell Reports 4 (2013), 124-134; Feiler et al., 2015; Porta et al., Nat. Comm., 2018). Further it has been observed that intracellular TDP-43 aggregates can be released in association with exosomes prior to spreading to the next cell (Nonaka et al., Cell Reports 4 (2013, 124-134)). Similarly, adenovirus-transduced TDP-43 expression leads to cytoplasmic aggregates which were phosphorylated and ubiquitinated, and, more importantly, were capable of acting as seeds for initiating cell-to-cell spreading (Ishii et al., PLoS ONE 12(6): e0179375, 2017). The patient-derived pathological TDP-43 can also lead to widespread deposition of endogenous TDP-43 following intracerebral inoculation into transgenic and wildtype mice (Porta et al., Nat. Comm., 2018).

### Differential Diagnostic Strategies for Neurodegenerative Diseases Including TDP-43 Proteinopathies

The diagnosis of FTD based on clinical manifestations is insufficient since the clinical representation can overlap with other diseases, particularly in the earlier stages. The development of biochemical biomarkers to distinguish different types of FTD pathology is therefore an important health-related goal. Development of antibodies against different conformations of TDP-43 will be an essential step in generating more sensitive and specific diagnostic tools. In parallel to biochemical biomarkers, the development of imaging biomarkers may enable early and specific detection of the pathology in TDP-43 proteinopathies. The ability to image TDP-43 deposition in the brain may be a substantial achievement for diagnosis and drug development for TDP-43 proteinopathies. The use of cell permeable antibody fragments could enable such detection.

The earliest event in neurodegenerative diseases is the acquisition of an alternative conformation that renders the protein toxic (aggregation). Moreover, this aggregated conformation can self-propagate by recruiting the endogenous, normal protein into the misfolded conformation as mechanistic basis for the observed spread through affected tissue.

Antibodies that allow the differential targeting of specific isoforms or post-translational modifications of TDP-43 offer many advantages since they can discriminate between the disease-associated and the functional, endogenous conformations of these proteins. These unique features offer the potential for the improvement of diagnostic and therapeutic applications since such antibodies can discriminate between previously inaccessible TDP-43 and TDP-43-derived targets. As such, these features are paramount to developing sensitive and specific diagnostics and therapeutics. The high-affinity and selective targeting of such TDP-43 subspecies is particularly important for TDP-43 proteinopathies, because TDP-43 aggregation can lead to dominant-negative effects on TDP-43 function, which could exacerbate disease if combined with the inadvertent targeting of functional TDP-43.

### Prevention and Treatment of TDP-43 Proteinopathies

TDP-43 aggregation and spreading of pathology are major hallmarks of ALS and FTD, fatal diseases for which currently no cure is available. Slowing or preventing these pathogenic processes using TDP-43-binding molecules has the potential to prevent or treat ALS, FTD and other TDP-43 proteinopathies mentioned above.

### Normal Cellular Functions and Distribution of TDP-43

Transactive response (TAR) DNA binding protein 43 kDa (TDP-43) is a 414-amino acid protein encoded by the TARDBP gene on chromosome 1p36.2 (also known as ALS10). Multiple TARDBP mRNAs are comprised by differential splicing of six (or potentially seven) exons, encoding a heterogeneous set of polypeptide isoforms with shared amino acid sequences (D'Alton et al., RNA, 2015). In the present application, TDP-43 refers to the human reference sequence (Q13148) and its isoforms, including polypeptides arising from differential splicing, proteolytic fragments and post-translationally modified polypeptides. TDP-43 belongs to the family of heterogeneous ribonucleoprotein (hnRNP) RNA binding proteins (Wang et al., Trends in Molecular Medicine Vol. 14 No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology 42 (2010) 1606-1609): two RNA recognition motifs (RRM1 and RRM2), which have two highly conserved hexameric ribonucleoprotein 2 (RNP2) and octameric ribonucleoprotein 1 (RNP1) regions, a nuclear export signal (NES) and a nuclear localization signal (NLS) enabling it to shuttle between the nucleus and the cytoplasm transporting bound mRNA, and a glycine-rich domain at the C-terminal, which mediates protein-protein interactions. TDP-43 is involved in multiple aspects of RNA processing, including transcription, splicing, transport, and stabilization (Buratti and Baralle, FEBS Journal 277 (2010) 2268-2281). It is a highly conserved, ubiquitously expressed protein with a tightly autoregulated expression and shuttling between the nucleus and cytoplasm. TDP-43 participates in multiple phases of RNA processing, including transcription, splicing, transport, and stabilization, all of which are crucial for neuronal function. Therefore, maintaining TDP-43 function is essential for producing structurally correct neuronal gene products and avoiding the production of potentially detrimental ones (including aggregate-prone forms of TDP-43 itself).

Intracellular elimination of TDP-43 and its aggregate-prone forms is accomplished by several different degradation pathways, including the ubiquitin proteasome system (UPS), the autophagy-lysosomal pathway (ALP), and chaperone mediated autophagy (CMA), as shown in both in vitro cell-cultures and in vivo studies (Wang et al., 2010, Ormeno et al., 2020). The efficacies of these intracellular mechanisms of eliminating TDP-43 are governed by the differences in TDP-43 isoform production and post-translational modifications. Of particular note, the burden of aggregated TDP-43 already present in the cell has a significant negative effect on its rate of elimination.

The possible role of TDP-43 in the pathology of neurodegenerative diseases has been reported with the accumulation of hyper-phosphorylated TDP-43 protein inclusions shown in subtypes of frontotemporal degeneration (FTD) and in most cases of amyotrophic lateral sclerosis (ALS; Arai et al., 2006; Neumann et al., 2006). In addition to FTD and ALS, further biochemical and immunohistochemical analyses have likewise demonstrated phospho-TDP-43 as a major constituent of cytoplasmic ubiquitin immunoreactive lesions in the cerebral cortex and hippocampus in patients with motor neuron disease (MND), Alzheimer's disease (AD), dementia with Lewy bodies, Parkinson's disease (PD), Progressive Supranuclear Palsy (PSP), Pick's disease (PiD), Multiple system Atrophy (MSA), and in patients with Huntington's disease (HD; Davidson et al., 2009). This pathology has been observed in both glia and neurons, with TDP-43 positive inclusions often, and at least partially, colocalized with the more characteristic inclusions that are found in these diseases, such as Tau, alpha-synuclein, beta-amyloid, and expanded polyglutamines (Amador-Oriz et al., 2007, Nakashima-Yasuda et al., 2007, Higashi et al., 2007, Schwab et al., 2008, Uryu et al., 2008).

### PRIOR ART

Patent application WO2009/008529 discloses antibody which binds specifically to an abnormal aggregate of TDP-43 protein.

Patent application WO2011005628A1 discloses antibody which bind to TDP-43 and methods of assessing the absence or presence of a neurodegenerative disease in a subject comprising characterizing TDP-43 in a tissue sample of the subject, wherein the tissue is cerebrospinal fluid.

Patent application WO2008/042190 discloses antibodies that bind to TDP-43 and methods of assessing the absence or presence of a neurodegenerative disease in a subject comprising characterizing TDP-43 in a tissue sample from said subject.

Patent application WO2020/234473 discloses TDP-43 specific binding molecules, in particular to anti-TDP-43 antibodies or an antigen-binding fragment or a derivative thereof and uses thereof, and methods to diagnose, prevent, alleviate and/or treat a disease, disorder and/or abnormality associated with TDP-43 aggregates including but not limited to Frontotemporal dementia (FTD), amyotrophic lateral sclerosis (ALS), Alzheimer's disease (AD), Parkinson's disease (PD), Chronic Traumatic Encelopathy (CTE), and limbic-predominant age-related TDP-43 encephalopathy (LATE).

### SUMMARY

In view of the foregoing, there is a need for TDP-43 binding molecules that selectively target specific TDP-43 isoforms and/or post-translationally modified forms of TDP-43 (i.e. phosphorylated forms) present in human tissues and biofluids. Such binding molecules can be used to detect, measure or eliminate TDP-43 species in situ in neuronal cells or TDP-43 species stored or released into other tissues or biological fluids. Moreover, they can be used as tools to evaluate or screen the activities of other TDP-43-binding molecules.

The development of sensitive and specific biofluid immunoassays using said TDP-43 binding molecules is an urgent task and will allow for the differentiation between individuals or groups of individuals with pathologies where TDP-43 proteinopathies play a role in disease etiology or disease progression. Similar assays are also imminently required for the evaluation of candidate therapeutic strategies targeting TDP-43 or TDP-43-associated pathways that can distinguish between disease-associated pathological processes and normal cellular functions. The ability to discriminate between health-promoting and pathological forms of TDP-43 is paramount, given that TDP-43 proteinopathies are largely accompanied by an overall loss of TDP-43 function.

The invention relates to TDP-43 binding molecules. The TDP-43 binding molecules herein described binds to an epitope within amino acids residues 361-414 of human TDP-43 (SEQ ID NO: 1) or to an equivalent epitope in non-human TDP-43. In one embodiment, the TDP-43 binding molecule binds to an epitope comprising at least one, two three, or four of the phosphorylated amino acids residues pS403, pS404, pS409 and/or pS410 of human TDP-43 (SEQ ID NO: 1) or to an equivalent epitope in non-human TDP-43. In another embodiment, the TDP-43 binding molecule binds to an epitope comprising at least one or two of the phosphorylated amino acids residues pS375 and/or pS379 of human TDP-43 (SEQ ID NO: 1) or to an equivalent epitope in non-human TDP-43. As used herein, phosphorylated serine residues are described as pS, with the amino acid residue numbered according to the human TDP-43 amino acid sequence, which is set forth in SEQ ID NO: 1. In one embodiment, the TDP-43 binding molecule binds to an epitope comprising phosphorylated amino acids residues pS403 and/or pS404, of human TDP-43 (SEQ ID NO: 1) or to an equivalent epitope in non-human TDP-43. In another embodiment, the TDP-43 binding molecule binds to an epitope comprising phosphorylated amino acids residues pS409 and/or pS410 of human TDP-43 (SEQ ID NO: 1) or to an equivalent epitope in non-human TDP-43. In yet another embodiment, the TDP-43 binding molecule binds to an epitope comprising phosphorylated amino acids residues pS375 and/or pS379 of human TDP-43 (SEQ ID NO: 1) or to an equivalent epitope in non-human TDP-43. Independent of the combination of one, two, three or four above listed characteristics, the binding molecules, preferably antibodies or antigen-binding fragments thereof, of the invention may reduce levels of TDP-43 within TDP-43-specific inclusion and/or phosphorylated TDP-43 in vivo.

The TDP-43 binding molecules of the invention are useful in drug screening assays. Thus the invention also provides for the use of TDP-43 binding molecules of the invention in assays or methods for screening TDP-43-targeted drugs, such as in assays or methods for the assessment of TDP-43-targeted drug efficacy.

The present invention is summarized in the following numbered embodiments:
1. A TDP-43 binding molecule which specifically binds phosphorylated TDP-43, wherein the binding molecule comprises:
   a) a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
   b) a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
   c) a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
   d) a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
   e) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
   f) a VH-CDR1 comprising the amino acid sequence of SEQ ID NO: 61, a VH-CDR2 comprising the amino acid sequence of SEQ ID NO: 62, VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
   g) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77.
1′. A TDP-43 binding molecule which specifically binds phosphorylated TDP-43, wherein the binding molecule comprises:
   a) a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
   b) a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
   c) a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16 and a VL-CDR3 comprising the amino acid sequence of SEQ ID NO: 37; or
   d) a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
   e) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
   f) a VH-CDR1 comprising the amino acid sequence of SEQ ID NO: 61, a VH-CDR2 comprising the amino acid sequence of SEQ ID NO: 62, VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
   g) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
   h) a VH-CDR1 comprising the amino acid sequence of SEQ ID NO: 111, a VH-CDR2 comprising the amino acid sequence of SEQ ID NO: 112, a VH-CDR3 comprising the amino acid sequence of SEQ ID NO: 113, a VL-CDR1 comprising the amino acid sequence of SEQ ID NO: 115, a VL-CDR2 comprising the amino acid sequence of SEQ ID NO: 116 and a VL-CDR3 comprising the amino acid sequence of SEQ ID NO: 117.
2. The TDP-43 binding molecule of embodiment 1, wherein the binding molecule according to any one of (a), (c), (f) or (g) binds TDP-43 positive inclusions.
3. The TDP-43 binding molecule of embodiment 2, wherein binding to TDP-43 positive inclusions is determined by immunohistochemistry.
4. The TDP-43 binding molecule of any one of the preceding embodiments, which binds to an epitope comprising phosphorylated amino acid residues pS403, pS404, pS409 and/or pS410 of human TDP-43 (SEQ ID NO: 1) or to an equivalent epitope in non-human TDP-43.
4′. The TDP-43 binding molecule of any one of embodiments 1 to 3, which binds to an epitope comprising phosphorylated amino acid residues pS375, pS379, pS403, pS404, pS409 and/or pS410 of human TDP-43 (SEQ ID NO: 1) or to an equivalent epitope in non-human TDP-43.
5. The TDP-43 binding molecule of any one of the preceding embodiments, which binds to an epitope comprising phosphorylated amino acid residues pS403 and/or pS404 of human TDP-43 (SEQ ID NO: 1) or to an equivalent epitope in non-human TDP-43.
6. The TDP-43 binding molecule of embodiment 5, which binds to the epitope with a KD of 13 nM or less, preferably 5 nM or less, preferably 2 nM or less, preferably wherein the epitope comprises or consists of GFNGGFG(pS)(pS)MDSKS (SEQ ID NO: 8) corresponding to amino acid positions 396 to 409 of SEQ ID NO: 1, more preferably wherein the KD is measured by surface plasmon resonance.
7. The TDP-43 binding molecule of any of embodiments 1 to 4, which binds to an epitope comprising phosphorylated amino acid residues pS409 and/or pS410 of human TDP-43 (SEQ ID NO: 1) or to an equivalent epitope in non-human TDP-43.
8. The TDP-43 binding molecule of embodiment 7, which binds the epitope with a KD of 3.5 nM or less, preferably 2.5 nM or less, preferably 1.7 nM or less, preferably wherein the epitope comprises or consists of FGSSMDSK(pS)(pS)GWG (SEQ ID NO: 9) corresponding to amino acid positions 401 to 413 of SEQ ID NO: 1, more preferably wherein the KD is measured by surface plasmon resonance.
8′. The TDP-43 binding molecule of any of embodiments 1 to 4, which binds to an epitope comprising phosphorylated amino acid residues pS375 and/or pS379 of human TDP-43 (SEQ ID NO: 1) or to an equivalent epitope in non-human TDP-43.
8″. The TDP-43 binding molecule of embodiment 8′, which binds the epitope with a KD of 21.5 nM or less, preferably wherein the epitope comprises or consists of GNNSY(pS)GSN(pS)GAAIG (SEQ ID NO: 5) corresponding to amino acid positions 370 to 384 of SEQ ID NO: 1, more preferably wherein the KD is measured by surface plasmon resonance.
9. A TDP-43 binding molecule, preferably which binds phosphorylated TDP-43, which comprises:
   a. a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
   b. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
   c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
   d. a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
   e. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
   f. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
   g. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, comprising a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
   h. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.
9′. A TDP-43 binding molecule, preferably which binds phosphorylated TDP-43, which comprises:
   a. a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
   b. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
   c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
   d. a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
   e. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
   f. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
   g. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, comprising a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
   h. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
   i. a VH-CDR1 comprising the amino acid sequence of SEQ ID NO: 111, a VH-CDR2 comprising the amino acid sequence of SEQ ID NO: 112, a VH-CDR3 comprising the amino acid sequence of SEQ ID NO: 113, a VL-CDR1 comprising the amino acid sequence of SEQ ID NO: 115, a VL-CDR2 comprising the amino acid sequence of SEQ ID NO: 116 and a VL-CDR3 comprising the amino acid sequence of SEQ ID NO: 117.
10. A TDP-43 binding molecule which binds TDP-43 positive inclusions, wherein the binding molecule comprises:
   a. a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
   b. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
   c. a VH-CDR1 comprising the amino acid sequence of SEQ ID NO: 61, a VH-CDR2 comprising the amino acid sequence of SEQ ID NO: 62, VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
   d. a VH-CDR1 comprising the amino acid sequence of SEQ ID NO: 51, a VH-CDR2 comprising the amino acid sequence of SEQ ID NO: 52, VH-CDR3 comprising the amino acid sequence of SEQ ID NO: 53, a VL-CDR1 comprising the amino acid sequence of SEQ ID NO: 75, a VL-CDR2 comprising the amino acid sequence of SEQ ID NO: 76, and a VL-CDR3 comprising the amino acid sequence of SEQ ID NO: 77; or
   e. a VH-CDR1 comprising the amino acid sequence of SEQ ID NO: 81, a VH-CDR2 comprising the amino acid sequence of SEQ ID NO: 82, VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16, and a VL-CDR3 comprising the amino acid sequence of SEQ ID NO: 87.
11. A TDP-43 binding molecule, which binds misfolded aggregated TDP-43 and non-aggregated physiological TDP-43, wherein the TDP-43 binding molecule comprises a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.
12. The TDP-43 binding molecule of embodiment 11, which binds to an epitope within amino acids residues 361-414 of human TDP-43 (SEQ ID NO: 1) or to an equivalent epitope in non-human TDP-43.
13. The TDP-43 binding molecule of any one of embodiments 11 or 12, which binds human TDP-43 (SEQ ID NO: 1) with a KD of 0.39 nM or less, preferably wherein the KD is measured by surface plasmon resonance.
14. The TDP-43 binding molecule of any of the preceding embodiments which comprises:
   a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10; and a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
   b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
   c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
   d. a Heavy Chain Variable Region (VH) comprising the sequence of SEQ ID NO: 40 or a Heavy Chain Variable Region (VH) having at 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
   e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 54; or
   f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; and a Light Chain Variable Region (VL) comprising the sequence of SEQ ID NO: 64; or
   g. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 74; or
   h. a Heavy Chain Variable Region (VH) comprising the sequence of SEQ ID NO: 80 or a Heavy Chain Variable Region (VH) having at least 94%, 95%, 96%, 97%, 98%, or 99% sequence identity to the amino acid sequence of SEQ ID NO: 80; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84.
14′. The TDP-43 binding molecule of any of the preceding embodiments which comprises:
   a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10; and a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
   b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
   c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
   d. a Heavy Chain Variable Region (VH) comprising the sequence of SEQ ID NO: 40 or a Heavy Chain Variable Region (VH) having at 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
   e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 54; or
   f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; and a Light Chain Variable Region (VL) comprising the sequence of SEQ ID NO: 64; or
   g. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 74; or
   h. a Heavy Chain Variable Region (VH) comprising the sequence of SEQ ID NO: 80 or a Heavy Chain Variable Region (VH) having at least 94%, 95%, 96%, 97%, 98%, or 99% sequence identity to the amino acid sequence of SEQ ID NO: 80; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
   i. a Heavy Chain Variable Region (VH) comprising the sequence of SEQ ID NO: 110 or a Heavy Chain Variable Region (VH) having at least 92%, 93%, 94%, 95%, 96%, 97%, 98%, or 99% sequence identity to the amino acid sequence of SEQ ID NO: 110; and a Light Chain Variable Region (VL) comprising the sequence of SEQ ID NO: 114.
15. The TDP-43 binding molecule of any one of the preceding embodiments, which is an antibody or an antigen-binding fragment thereof.
16. The TDP-43 binding molecule of any one of the preceding embodiments, which is an IgA, IgD, IgE, IgM, IgG1, IgG2, IgG2a, IgG2b, IgG3 or IgG4 antibody or antigen-binding fragment thereof.
17. The TDP-43 binding molecule of any of the preceding embodiments, wherein the binding molecule is an immunoconjugate.
18. The TDP-43 binding molecule of embodiment 17, wherein the immunoconjugate comprises paramagnetic beads.
19. The TDP-43 binding molecule of embodiment 17, wherein the immunoconjugate comprises biotin.
20. The TDP-43 binding molecule of embodiment 17, wherein the immunoconjugate comprises an additional therapeutic molecule.
21. The TDP-43 binding molecule of any one of the preceding embodiments for use in human or veterinary therapy and/or diagnosis.
22. The TDP-43 binding molecule for use of embodiment 21, wherein the TDP-43 binding molecule is a therapeutic or a diagnostic tool.
23. The TDP-43 binding molecule of any one of embodiments 1 to 20 for research use, in particular as an analytical tool or a reference molecule.
24. The TDP-43 binding molecule of any one of embodiments 1 to 20 for use in the prevention, alleviation, treatment and/or diagnosis of diseases, disorders and/or abnormalities associated with TDP-43.
25. The TDP-43 binding molecule of any one of embodiments 1 to 20 for use in the prevention, alleviation, treatment and/or diagnosis of a TDP-43 proteinopathy.
26. The TDP-43 binding molecule for use of embodiment 25, wherein the TDP-43 binding molecule is used as a diagnostic tool to diagnose or monitor a TDP-43 proteinopathy.
27. The TDP-43 binding molecule for use according to any one of embodiments 25 or 26, wherein the TDP-43 proteinopathy is either:
   a. a disease, disorder and/or abnormality associated with TDP-43 aggregates selected from the group consisting of Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy; or
   b. a disease arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.
28. The TDP-43 binding molecule for use according to any one of embodiments 25 to 27, wherein the TDP-43 proteinopathy is either:
   a. a disease, disorder and/or abnormality associated with TDP-43 aggregates selected from the group consisting of Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Alzheimer's Disease (AD), Down Syndrome (DS), Parkinson's Disease (PD) and related disorders (including PD with Dementia (PDD), dementia with Lewy Bodies (DLB), multiple system atrophy (MSA)), limbic-predominant age-related TDP-43 encephalopathy (LATE), myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy; or
   b. a disease arising from mutations or variant-associated risk alleles of the progranulin (GRN), TARDBP, C9ORF72, valosin-containing protein (VCP), angiogenin (ANG), desmin (DES), myotilin (MYOT), TMEM106B genes.
29. The TDP-43 binding molecule for use according to any one of embodiments 25 to 28, wherein the disease, disorder and/or abnormality associated with TDP-43, or TDP-43 proteinopathy, is amyotrophic lateral sclerosis (ALS).
30. The TDP-43 binding molecule for use according to any one of embodiments 25 to 28, wherein the disease, disorder and/or abnormality associated with TDP-43, or TDP-43 proteinopathy, is Alzheimer's disease (AD).
31. The TDP-43 binding molecule for use according to any one of embodiments 25 to 28, wherein the disease, disorder, and/or abnormality associated with TDP-43, or TDP-43 proteinopathy, is Frontotemporal dementia (FTD).
32. The TDP-43 binding molecule for use according to embodiments 25 to 28, wherein the disease, disorder and/or abnormality associated with TDP-43, or TDP-43 proteinopathy, is limbic-predominant age-related TDP-43 encephalopathy (LATE).
33. The TDP-43 binding molecule for use according to any one of embodiments 25 to 28, wherein the disease, disorder and/or abnormality associated with TDP-43, or TDP-43 proteinopathy, is Frontotemporal Lobar Degeneration with Motor Neuron Disease FTLD-MND.
34. A pharmaceutical composition comprising the TDP-43 binding molecule of any one of embodiments 1 to 20 and a pharmaceutically acceptable carrier and/or excipient.
34′. A diagnostic composition comprising the TDP-43 binding molecule of any one of embodiments 1 to 20 and an acceptable carrier and/or excipient.
35. A nucleic acid molecule encoding the TDP-43 binding molecule of any one of embodiments 1 to 20.
35′. The nucleic acid of embodiment 35, comprising the nucleotide sequence set forth as SEQ ID NO: 18, SEQ ID NO: 19, SEQ ID NO: 28, SEQ ID NO: 29, SEQ ID NO: 38, SEQ ID NO: 39, SEQ ID NO: 48, SEQ ID NO: 49, SEQ ID NO: 58, SEQ ID NO: 59, SEQ ID NO: 68, SEQ ID NO: 69, SEQ ID NO: 79, SEQ ID NO: 88, SEQ ID NO: 89, SEQ ID NO: 118 or SEQ ID NO: 119.
36. A recombinant vector comprising the nucleic acid of embodiment 35.
37. A host cell comprising the nucleic acid of embodiment 35 and/or the vector of embodiment 36.
38. A host cell that expresses a TDP-43 binding molecule according to any one of embodiments 1 to 20.
39. An expression vector comprising the nucleic acid molecule of embodiment 35.
40. A cell-free expression system containing the expression vector of embodiment 39.
41. A method for producing a TDP-43 binding molecule, in particular an antibody or antigen-binding fragment thereof, comprising the steps of:
   a. culturing the host cell of embodiment 37 or 38 or cell-free expression system of embodiment 40 under conditions suitable for producing the binding molecule, in particular the antibody or antigen-binding fragment thereof; and
   b. isolating the binding molecule, in particular the antibody or antigen-binding fragment thereof.
41′. The TDP-43 binding molecule of any one of embodiments 1 to 20, for use in the detection and/or quantification of TDP-43 in a sample, wherein the sample is saliva, urine, nasal secretion, blood (including whole blood, plasma and serum, platelets rich plasma, platelets cytosol fraction), brain and/or CSF sample, brain and/or ISF sample, more particularly blood, brain, CSF and/or ISF sample.
42′. The TDP-43 binding molecule of any one of embodiments 1 to 20, for use in the detection and/or quantification of phosphorylated TDP-43 in a sample, wherein the sample is saliva, urine, nasal secretion, blood (including whole blood, plasma and serum, platelets rich plasma, platelets cytosol fraction), brain and/or CSF sample, brain and/or ISF sample, more particularly blood, brain, CSF and/or ISF sample.
42. Use of a TDP-43 binding molecule of any one of embodiments 1 to 20 in a pairing assay comprising the steps of:
   a. Incubating a sample with a capture antibody and a detect antibody;
   b. Incubating the mixture obtained in step a with a reagent suitable for detection by the detect antibody;
   c. Measuring the signal emitted by the detect antibody;
   wherein the capture antibody is selected from an antibody as defined in any one of embodiments 1 to 20.
43. The use of a TDP-43 binding molecule of embodiment 42, wherein the detect antibody is selected form an antibody as defined in any one of embodiments 1 to 20.
44. The use of a TDP-43 binding molecule of embodiment 42 or 43, wherein the reagent is Streptavidin-β-D-Galactosidase.
45. The use of a TDP-43 binding molecule of any one of embodiments 42 to 44, wherein the capture antibody and/or detect antibody binds phosphorylated TDP-43, preferably specifically binds phosphorylated TDP-43.
46. The use of a TDP-43 binding molecule of any one of embodiments 42 to 44, wherein the capture antibody and/or detect antibody binds misfolded aggregated TDP-43 and non-aggregated physiological TDP-43.
47. The use of a TDP-43 binding molecule of any one of embodiment 42 to 46, wherein the capture antibody comprises:
   i. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
   ii. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
   iii. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.
48. The use of a TDP-43 binding molecule of embodiment 42 to 47, wherein the detect antibody comprises:
   i. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
   ii. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
   iii. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.
48′. The use of a TDP-43 binding molecule of embodiments 42 to 47, wherein the detect antibody comprises:
   i. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
   ii. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
   iii. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107; or
   iv. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
   v. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57.
49. The use of a TDP-43 binding molecule of embodiment 47 or 48 wherein the detect antibody comprises:
   a. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87;
and the capture antibody comprises:
   b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.
49′. The use of a TDP-43 binding molecule of embodiment 47 or 48 wherein the detect antibody comprises:
   a. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27;
and the capture antibody comprises:
   b. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97.
49″. The use of a TDP-43 binding molecule of embodiment 47 or 48 wherein the detect antibody comprises:
   a. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27;
and the capture antibody comprises:
   b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.
49′″. The use of a TDP-43 binding molecule of embodiment 47 or 48 wherein the detect antibody comprises:
   a. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57;
and the capture antibody comprises:
   b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.
50. A method of quantifying phosphorylated TDP-43 in a sample, the method comprising contacting the sample with a TDP-43 binding molecule according to any one of embodiments 1 to 20.
51. The method of quantifying of embodiment 50, wherein the sample was obtained from a subject.
51′. The method of embodiment 51, wherein the sample is human blood, cerebrospinal fluid (CSF), interstitial fluid (ISF), saliva, nasal secretion and/or urine, preferably blood, CSF or ISF.
52. The method of quantifying of embodiment 50 or 51, further comprising comparing detected TDP-43 levels from the sample to a control.
53. The method of quantifying of embodiment 52, wherein the control comprises phosphorylated TDP-43.
54. The method of quantifying of embodiment 52 or 53, wherein the control was determined using a known amount of calibrator for phosphorylated TDP-43.
55. A method of quantifying phosphorylated TDP-43 in a sample obtained from a subject, the method comprising the use of the TDP-43 binding molecule of any one of embodiments 42 to 49.
55′. A method for monitoring a disease, disorder and/or condition associated with TDP-43 at two or more time points using samples from a subject comprising contacting the samples with a TDP-43 binding molecule of any of embodiments 1 to 20, wherein;
   a. higher levels of TDP-43 and/or phosphorylated TDP-43 in the later sample compared with one or more earlier samples are indicative of progression of a disease, disorder and/or condition associated with TDP-43; or
   b. lower levels of TDP-43 and/or phosphorylated TDP-43 in the later sample compared with one or more earlier samples are indicative of regression of a disease, disorder and/or condition associated with TDP-43; or
   c. no significant change of levels of TDP-43 and/or phosphorylated TDP-43 in the later sample compared with one or more earlier samples are indicative of lack of progression of a disease, disorder and/or condition associated with TDP-43.
55″. A method for monitoring a disease, disorder and/or condition associated with TDP-43 at two or more time points using samples from a subject comprising contacting the samples with a TDP-43 binding molecule of any of embodiments 1 to 20, wherein;
   a. higher levels of TDP-43 and/or phosphorylated TDP-43 in the later sample compared with one or more earlier samples are indicative of regression of a disease, disorder and/or condition associated with TDP-43; or
   b. lower levels of TDP-43 and/or phosphorylated TDP-43 in the later sample compared with one or more earlier samples are indicative of progression of a disease, disorder and/or condition associated with TDP-43; or
   c. no significant change of levels of TDP-43 and/or phosphorylated TDP-43 in the later sample compared with one or more earlier samples are indicative of lack of progression of a disease, disorder and/or condition associated with TDP-43.
55′″. The method of embodiments 55′ or 55″, performed at multiple time points in matched samples between the treatment and placebo groups in order to monitor the effectiveness of the candidate therapy over a defined time period.
56. A kit for diagnosis of a disease, disorder and/or abnormality associated with TDP-43, or a TDP-43 proteinopathy, or for use in a method of any one of claims 50 to 55, comprising a TDP-43 binding molecule according to any one of embodiments 1 to 20.
57. The kit of embodiment 56 comprising a TDP-43 binding molecule according to any one of embodiments 1 to 20 as capture antibody and a different TDP-43 binding molecule according to any one of embodiments 1 to 20 as detect antibody.
58. The kit of embodiment 57 wherein the kit further comprises magnetic particles to which the capture antibody is, or can be attached.
59. The kit of embodiment 57 or 58, wherein the detect antibody is labelled, either directly or indirectly.
60. The kit of any one of embodiments 57 to 59, further comprising a container that contains the TDP-43 binding molecule(s).

For the avoidance of doubt, where reference is made to a numbered embodiment this is intended to also encompass reference to the equivalent prime (′) embodiment (e.g. reference to embodiment 1 encompasses 1 and 1′).

In some embodiments, the invention encompasses binding molecules, particularly antibodies and antigen-binding fragments thereof of the invention as described herein that specifically bind phosphorylated TDP-43 and the use of these binding molecules to diagnose, prevent, alleviate and/or treat a disease, disorder and/or abnormality associated with TDP-43 aggregates, or TDP-43 proteinopathies including, but not limited to, Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy, diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes. Preferably, the use of these binding molecules to diagnose, prevent, alleviate and/or treat a disease, disorder and/or abnormality associated with TDP-43, in particular associated with TDP-43 aggregates, or TDP-43 proteinopathy is directed to Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Alzheimer's Disease (AD), Down Syndrome (DS), Parkinson's Disease (PD) and related disorders (including PD with Dementia (PDD), dementia with Lewy Bodies (DLB), multiple system atrophy (MSA)), limbic-predominant age-related TDP-43 encephalopathy (LATE), myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy, and diseases of both sporadic and hereditary origins, including genetic cases arising from mutations or variant-associated risk alleles of the progranulin (GRN), TARDBP, C9ORF72, valosin-containing protein (VCP), angiogenin (ANG), desmin (DES), myotilin (MYOT), TMEM106B genes. More preferably, the use is directed to amyotrophic lateral sclerosis (ALS). More preferably, the use is directed to Frontotemporal dementia (FTD). More preferably, the use is directed to limbic-predominant age-related TDP-43 encephalopathy (LATE). More preferably, the use is directed to Alzheimer's disease (AD). More preferably, the use is directed to Frontotemporal Lobar Degeneration with Motor Neuron Disease FTLD-MND (also known as ALS-FTD).

In another embodiment, a TDP-43 binding molecule, particularly a anti TDP-43 antibody or an antigen-binding fragment thereof of the invention (as described herein specific for TDP-43) is contacted with a sample to detect, diagnose and/or monitor a disease, disorder and/or abnormality associated with TDP-43, in particular associated with aggregated or aggregate-prone forms of TDP-43 (as differentiated from normal, functional TDP-43) in a suspected TDP-43 or other proteinopathy, particularly including the TDP-43-associated proteinopathies including but not limited to Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy, or diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes and differentiation of these versus non-TDP-43-associated clinical phenocopies.

In one embodiment, the invention encompasses binding molecules, particularly antibodies or antigen-binding fragments thereof of the invention as described herein that specifically bind phosphorylated TDP-43 and the use of these binding molecules, particularly of these antibodies, to detect the presence of specific phosphorylated TDP-43-derived species in a sample. Accordingly, TDP-43 binding molecules of the invention, such as, anti-TDP43 antibodies as described herein, can be used, inter alia, to screen a clinical sample, in particular human blood, cerebrospinal fluid (CSF), interstitial fluid (ISF), saliva, nasal secretion, and/or urine for the presence of TDP-43 in samples, for example, by using an ELISA-based or surface adapted assay. Tissue samples may be used in some circumstances, such as brain tissue samples. The methods and compositions of the invention also have applications in diagnosing presymptomatic disease and/or in monitoring disease progression and/or therapeutic efficacy. According to some embodiments, an antibody specific for TDP-43 (e.g., a full-length antibody or a TDP-43 binding fragment or derivative of an antibody) is contacted with a sample (e.g., blood, cerebrospinal fluid (CSF), interstitial fluid (ISF), saliva, nasal secretion, urine or brain tissue) to detect, Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy. In one embodiment, an antibody specific for TDP-43 (e.g., a full-length antibody or a TDP-43 binding fragment or derivative of an antibody) is contacted with a sample (e.g., blood, cerebrospinal fluid (CSF), interstitial fluid (ISF), saliva, nasal secretion, urine or brain tissue) to detect, diagnose and/or monitor diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

The TDP-43 binding molecules of the invention may be used to detect and/or quantify TDP-43, preferably phosphorylated TDP-43 in suitable samples. The samples may be acellular samples, such as blood, CSF, ISF, saliva, nasal secretion or urine. In a preferred embodiment the acellular samples may be blood, CSF or ISF. “Blood” includes whole blood and derivative samples, such as serum and plasma. “Plasma” includes derivative samples, such as platelets. “Platelets” includes derivative samples, such as platelets cytosol fraction. The samples may be cellular samples, such as tissue, preferably brain tissue. An appropriate TDP-43 species is quantified or detected. In one embodiment, soluble TDP-43 or phospho-TDP-43 is quantified or detected in an acellular sample (such as the detection of TDP-43 from human plasma in Example 8). In one embodiment, soluble TDP-43 or phospho-TDP-43 is quantified or detected in an acellular sample (such as the detection of TDP-43 from platelet fractions derived from human plasma in Example 9). In another embodiment, TDP-43 inclusions are quantified or detected in a cellular sample, preferably brain tissue (such as the detection of phospho-TDP-43 inclusions in Example 7).

The TDP-43 binding molecules of the invention may be used to quantify phosphorylated TDP-43 in suitable samples, in particular clinical samples such as blood, CSF, ISF, saliva, nasal secretion or urine. Many suitable immunoassay formats are known. Thus, the methods (such as ELISA, MSD (Meso Scale Discovery), HTRF (Homogeneous Time Resolved Fluorescence), SIMOA®, and AlphaLISA) may be performed for diagnostic purposes. It is demonstrated herein that the binding molecules of the invention have phospho-site specificity, high affinity, binding to human TDP-43 pathology in brain tissues, utility in high-sensitivity immunoassays. Methods of quantifying phosphorylated TDP-43 in suitable samples using binding molecules of the invention may also be used to select a therapy (for further treatment of the subject). Thus, personalized treatment methods are envisaged. A sample may be taken before and after treatment according to such methods.

In some embodiments, the TDP-43 binding molecules of the invention are used in a pairing assay comprising the steps of incubating a sample with a capture antibody comprising a TDP-43 binding molecule described herein conjugated to paramagnetic beads and a detect antibody comprising a TDP-43 binding molecule described herein conjugated to biotin; incubating the obtained solution with a reagent suitable for detection by the detect antibody, such as Streptavidin-β-D-Galactosidase; measuring the signal emitted by the detect antibody. In some embodiment, the capture antibody or the detect antibody is a TDP-43 binding molecule which binds misfolded aggregated TDP-43 and non-aggregated physiological TDP-43. In some embodiments, the capture antibody or the detect antibody binds specifically phosphorylated TDP-43. In one embodiment, the capture antibody binds misfolded aggregated TDP-43 and non-aggregated physiological TDP-43 and the detect antibody binds specifically phosphorylated TDP-43. In another embodiment, the detect antibody binds misfolded aggregated TDP-43 and non-aggregated physiological TDP-43 and the capture binds specifically phosphorylated TDP-43

In other embodiments, the invention provides methods for preventing, alleviating and/or treating a disease, disorder and/or abnormality associated with TDP-43, in particular associated with TDP-43 aggregates, or TDP-43 proteinopathy. According to one embodiment, the methods of the invention comprise administering an effective concentration of a binding molecule, particularly an antibody of the invention specific for TDP-43 (e.g., a full-length antibody or a TDP-43 binding fragment or derivative of an antibody) as described herein to a subject. In another embodiment, the invention provides a method for preventing, alleviating and/or treating a TDP-43 proteinopathy. According to some embodiments, a binding molecule, particularly an antibody of the invention or an antigen-binding fragment thereof as described herein specific for phosphorylated TDP-43 is administered to treat, alleviate and/or prevent amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND. In another embodiment, a binding molecule, particularly an antibody of the invention or an antigen-binding fragment thereof as described herein specific for phosphorylated TDP-43 is administered to prevent, alleviate and/or treat a neurodegenerative disease selected from amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND.

In another embodiment, a binding molecule, particularly an antibody of the invention or antigen-binding fragment thereof as described herein specific for TDP-43 is administered to prevent, alleviate and/or treat a disease selected from: Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy. In another embodiment, a binding molecule, particularly an antibody of the invention or antigen-binding fragment thereof as described herein specific for TDP-43 is administered to prevent, alleviate and/or treat a disease arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

### DETAILED DESCRIPTION OF EMBODIMENTS OF THE INVENTION

### Definitions

Transactive response (TAR) DNA binding protein 43 kDa (herein referred as TDP-43) is a 414-amino acid protein encoded by the TARDBP gene on chromosome 1p36.2 (also known as ALS10). Multiple TARDBP mRNAs are comprised by differential splicing of six (or potentially seven) exons, encoding a heterogeneous set of polypeptide isoforms with shared amino acid sequences (d'Alton et al., RNA). TDP-43 belongs to the family of heterogeneous ribonucleoprotein (hnRNP) RNA binding proteins (Wang et al., Trends in Molecular Medicine Vol. 14 No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology 42 (2010) 1606-1609): two RNA recognition motifs (RRM1 and RRM2), which have two highly conserved hexameric ribonucleoprotein 2 (RNP2) and octameric ribonucleoprotein 1 (RNP1) regions, a nuclear export signal (NES) and a nuclear localization signal (NLS) enabling it to shuttle between the nucleus and the cytoplasm transporting bound mRNA, and a glycine-rich domain at the C-terminal, which mediates protein-protein interactions. TDP-43 is involved in multiple aspects of RNA processing, including transcription, splicing, transport, and stabilization (Buratti and Baralle, FEBS Journal 277 (2010) 2268-2281). It is a highly conserved, ubiquitously expressed protein with a tightly autoregulated expression and shuttling between the nucleus and cytoplasm.

In the present application, TDP-43 refers to the human reference sequence (Q13148) and its isoforms, including proteolytic fragments and post-translationally modified polypeptides that retain the epitope bound by the binding molecule. Human TDP-43 has the following sequence:

(SEQ ID NO: 1)
MSEYIRVTEDENDEPIEIPSEDDGTVLLSTVTAQFPGACGLRYRNPVS
QCMRGVRLVEGILHAPDAGWGNLVYVVNYPKDNKRKMDETDASSAVKV
KRAVQKTSDLIVLGLPWKTTEQDLKEYFSTFGEVLMVQVKKDLKTGHS
KGFGFVRFTEYETQVKVMSQRHMIDGRWCDCKLPNSKQSQDEPLRSRK
VFVGRCTEDMTEDELREFFSQYGDVMDVFIPKPFRAFAFVTFADDQIA
QSLCGEDLIIKGISVHISNAEPKHNSNRQLERSGREGGNPGGFGNQGG
FGNSRGGGAGLGNNQGSNMGGGMNFGAFSINPAMMAAAQAALQSSWGM
MGMLASQQNQSGPSGNNQNQGNMQREPNQAFGSGNNSYSGSNSGAAIG
WGSASNAGSGSGFNGGFGSSMDSKSSGWGM.

An “antigen binding molecule,” as used herein, is any molecule that can specifically or selectively bind to an antigen, in particular TDP-43. A binding molecule may include or be an antibody or a fragment thereof. An anti-TDP-43 binding molecule is a molecule that binds to the TDP-43 protein, such as an anti-TDP-43 antibody or fragment thereof, at a specific recognition site, epitope. That is, antigen-binding molecules of the invention bind to an epitope within the amino acid sequence of SEQ ID NO: 1. The antigen-binding molecules, in particular antibodies or antigen-binding fragments thereof provided herein, recognize full-length TDP-43. Other anti-TDP-43 binding molecules may also include multivalent molecules, multi-specific molecules (e.g., diabodies), fusion molecules, aptamers, avimers, or other naturally occurring or recombinantly created molecules. Illustrative antigen-binding molecules useful in the present invention include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

The terms “anti TDP-43 antibody” and “an antibody that binds to TDP-43” or simply “antibody” as used herein refer to an antibody that is capable of binding TDP-43 with sufficient affinity such that the antibody is useful as a diagnostic and/or therapeutic agent in targeting TDP-43. In general, the term “antibody” is used herein in the broadest sense and encompasses various antibody structures, including but not limited to monoclonal antibodies, polyclonal antibodies, multispecific antibodies (e.g., bispecific or biparatopic antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, antibodies or antibodies displayed upon the surface of a phage or displayed upon the surface of a chimeric antigen receptor (CAR) T cell.

An “antigen-binding fragment” of an antibody, or “functional fragment thereof” refers to a molecule other than an intact, or full-length, antibody that comprises a portion of an intact, or full-length, antibody and that binds (fully or partially) the antigen to which the intact, or full-length, antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Antigen-binding fragments may also be referred to as “functional fragments” as they retain the binding function of the original antibody from which they are derived.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of TDP-43. Said binding/interaction is also understood to define a “specific recognition”. The term “specifically recognizing” means in accordance with this invention that the antibody is capable of specifically interacting with and/or binding to at least two amino acids of TDP-43 as defined herein, in particular interacting with/binding to at least two amino acids within amino acids residues 361-414 of human TDP-43 (SEQ ID NO: 1).

The term “pan TDP-43 antibody” refers to an antibody that binds to misfolded aggregated TDP-43 and non-aggregated physiological TDP-43, including monomeric TDP-43, oligomeric TDP-43, post-translationally modified TDP-43 (such as phosphorylated, ubiquitinated, acetylated, sumoylated, and/or methylated), aggregated TDP-43 and truncated TDP-43.

The terms “specific interaction”, “specific binding” and “specifically binds”, as used interchangeably in accordance with the present invention, mean that the antibody or antigen-binding fragment thereof of the invention does not or does not essentially cross-react with (poly)peptides of similar structures. Thus, a TDP-43 binding molecule of the invention which specifically binds phosphorylated TDP-43 at one or more phosphorylated residues does not or does not essentially cross-react with TDP-43 that is not phosphorylated at those one or more residues. Accordingly, in some embodiments of the invention, the antibody or antigen-binding fragment thereof of the invention specifically binds to/interacts with structures of TDP-43 formed by particular phosphorylated amino acid sequences within amino acids residues of human TDP-43 (SEQ ID NO: 1), more particularly binds to/interacts with structures of TDP-43 formed by particular amino acid sequences within phosphorylated amino acids residues pS403, pS404, pS409 or pS410 of human TDP-43 (SEQ ID NO: 1), even more particularly binds to/interacts with binding to at least one or two phospho-serine selected from the group consisting of pS403, pS404, pS409, pS410 of human TDP-43 (SEQ ID NO: 1). In such embodiments, the antibody or antigen-binding fragment thereof of the invention does not or does not essentially cross-react with non-phosphorylated S403, S404, S409 or S410 of human TDP-43 (SEQ ID NO: 1).

In some embodiments of the invention, the antibody or antigen-binding fragment thereof of the invention specifically binds to/interacts with structures of TDP-43 formed by particular phosphorylated amino acid sequences within amino acids residues of human TDP-43 (SEQ ID NO: 1), more particularly binds to/interacts with structures of TDP-43 formed by particular amino acid sequences within phosphorylated amino acids residues pS375 or pS379 of human TDP-43 (SEQ ID NO: 1), even more particularly binds to/interacts with binding to at least one phospho-serine selected from the group consisting of pS375 or pS379 of human TDP-43 (SEQ ID NO: 1). In such embodiments, the antibody or antigen-binding fragment thereof of the invention does not or does not essentially cross-react with non-phosphorylated S375 or S379 of human TDP-43 (SEQ ID NO: 1).

In some embodiments, the TDP-43 binding molecule binds both to misfolded aggregated TDP-43 and non-aggregated physiological TDP-43, including monomeric TDP-43, oligomeric TDP-43, post-translationally modified TDP-43 (such as phosphorylated, ubiquitinated, acetylated, sumoylated, and/or methylated), aggregated TDP-43 and truncated TDP-43. In some embodiment of the invention, the antibody or antigen-binding fragment thereof of the invention specifically binds to/interacts with structures of TDP-43 formed by particular amino acid sequences within amino acids residues 361 to 375 of human TDP-43 (SEQ ID NO: 1).

Inclusions relate to diverse intracellular non-living substances that are not bound by membranes. Inclusions may comprise stored nutrients/deutoplasmic substances, secretory products, and pigment granules. “TDP-43 Positive-inclusions” or “positively stained inclusions” refers to pathogenic deposits of TDP-43 carrying disease-specific post-translational modifications, such as phosphorylation, ubiquitination, sumoylation and acetylation. The pathogenic deposits of TDP-43 can comprise full-length and/or specific truncated forms of the protein.

The TDP-43 binding molecules herein described bind to TDP-43 positive inclusions, in particular to TDP-43 positive inclusions comprising, consisting of or essentially consisting of phosphorylated TDP-43. The TDP-43 positive inclusions may be cytoplasmic inclusions. Reference may be made to Examples 7 for a detailed description of staining assay that may be employed on TDP-43 positive inclusions.

Cross-reactivity of antigen-binding molecules, in particular a panel of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to certain structure of TDP-43 as defined herein, e.g., a specific epitope or (poly)peptide/protein of TDP-43 as defined herein but do not or do not essentially bind to any of the other epitopes or (poly)peptides of the same TDP-43, are considered specific for the epitope or (poly)peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIACORE™), analytical ultracentrifugation, isothermal titration calorimetry, fluorescence anisotropy, fluorescence spectroscopy or by radiolabeled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The term “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also, phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant (human) antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

“Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones et al., Nature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in the context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins.

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Humanized antibodies, humanized antibody constructs, humanized antibody fragments, humanized antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., Molecular Cloning: A Laboratory Manual; Cold Spring Harbor Laboratory Press, 2^{nd} edition (1989) and 3^{rd} edition (2001). The term “Ig-derived domain” particularly relates to (poly)peptide constructs comprising at least one CDR. Fragments or derivatives of the recited Ig-derived domains define (poly)peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2^{nd} edition (1989) and 3^{rd} edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5^{th} edit. NIH Publication no. 91-3242 U.S. Department of Health and Human Services (1991). CDR sequences provided herein are defined according to Kabat. However, it will be understood by the skilled person that the invention is intended to encompass binding molecules in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V, Chaume D, Bodmer J, Müller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc M P. Immunol Today. 1997 November; 18(11):509), MacCallum (MacCallum R M, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9. 10.1016/j.molimm.2008.05.022) numbering schemes may be adopted in order to define the CDRs.

Accordingly, in the context of the present invention, the antibody molecule described herein above is selected from the group consisting of a full antibody (immunoglobulin, like an IgG1, an IgG2, an IgA1, an IgA2, an IgG3, an IgG4, an IgA, an IgM, an IgD or an IgE), F(ab)-, Fab′-SH-, Fv-, Fab′-, F(ab′)2-fragment, a chimeric antibody, a CDR-grafted antibody, a fully human antibody, a bivalent antibody-construct, an antibody-fusion protein, a synthetic antibody, a bivalent single chain antibody, a trivalent single chain antibody and a multivalent single chain antibody.

“Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art (see e.g. LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules or antigen-binding fragments thereof are provided that can successfully be employed in pharmaceutical compositions.

The specificity of the antibody or antigen-binding fragment of the present invention may not only be expressed by the nature of the amino acid sequence of the antibody or the antigen-binding fragment as defined above but also by the epitope to which the antibody is capable of binding. Thus, the present invention relates, in one embodiment, to an anti-TDP-43 antibody or an antigen-binding fragment thereof, in particular an anti-phosphorylated TDP-43 antibody or an antigen-binding fragment thereof, which recognizes the same epitope as an antibody of the invention.

It may be understood by a person skilled in the art that the epitopes may be comprised in the TDP-43 protein, but may also be comprised in a degradation product thereof or may be a chemically synthesized peptide. The amino acid positions are only indicated to demonstrate the position of the corresponding amino acid sequence in the sequence of the (full length human) TDP-43 protein (SEQ ID NO: 1). The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or non-natural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane.

In order to test whether an antibody in question and the antibody of the present invention recognize the same epitope, the following competition study may be carried out: Vero cells infected with 3 MOI (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional (inversely proportional) to the concentration of the antibody in question is indicative that both antibodies recognize the same epitope. However, many other assays are known in the art which may be used.

The present invention also relates to the production of specific antibodies against native polypeptides and recombinant polypeptides of phosphorylated TDP-43. This production is based, for example, on the immunization of animals, like mice. However, also other animals for the production of antibody/antisera are envisaged within the present invention. For example, monoclonal and polyclonal antibodies can be produced by rabbits, mice, goats, donkeys and the like. The polynucleotide encoding a correspondingly chosen polypeptide of TDP-43 can be subcloned into an appropriate vector, wherein the recombinant polypeptide is to be expressed in an organism capable of expression, for example in bacteria. Thus, the expressed recombinant protein can be intra-peritoneally injected into mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of phosphorylated TDP-43, in particular the epitope of the antibodies provided herein, can be obtained by directly immunizing the animal by directly injecting intramuscularly the vector expressing the desired polypeptide or a protein or an epitope of phosphorylated TDP-43, in particular the epitope of the antibodies of the invention, which lies within amino acid residues 361-414 of human TDP-43 (SEQ ID NO:1), more particularly an epitope which binds to/interacts with structures of TDP-43 formed by particular amino acid sequences within phosphorylated amino acids residues pS403, pS404, pS409, pS410 of human TDP-43 (SEQ ID NO: 1).

The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

Thus, under designated assay conditions, the specified antibodies and the corresponding epitope of TDP-43 bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell, for a description of immunoassay formats and conditions that can be used to determine specific immunoreactivity. Typically, a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. Cit.).

The “class” of an antibody refers to the type of constant domain or constant region possessed by Its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure.

In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; see Edelman, G. M. et al., Proc. Natl. Acad. USA, 63, 78-85 (1969)); however, Asn297 may also be located about ±3 amino acids upstream or downstream of position 297, i.e., between positions 294 and 300, due to minor sequence variations in antibodies. Such fucosylation variants may have improved ADCC function.

See, e.g., US Patent Publication Nos. US 2003/0157108 (Presta, L.); US 2004/0093621 (Kyowa Hakko Kogyo Co., Ltd). Examples of publications related to “defucosylated” or “fucose deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and W02003/085 107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al., Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Alternatively, non-radioactive assays methods may be employed (see, for example, ACTI™ non radioactive cytotoxicity assay for flow cytometry (CellTechnology, Inc. Mountain View, CA; and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” FC mutant with substitution of residues 265 to alanine and 297 to glycine. Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908). Other known mutations at position 234, 235 and 321, the so-called TM mutant containing mutations L234F/L235E/P331S in the CH2 domain, can be used (Oganesyan et al. Acta Cryst. D64, 700-704. (2008)). Antibodies from the human IgG4 isotype include mutations S228P/L235E to stabilize the hinge and to reduce FgR binding (Schlothauer et al, PEDS, 29 (10):457-466).

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Antibodies may be produced using recombinant methods and compositions, e.g., as described in U.S. Pat. No. 4,816,567. In one embodiment, isolated nucleic acid encoding an anti-phosphorylated TDP-43 antibody described herein is provided. Such nucleic acid may encode an amino acid sequence comprising the VL and/or an amino acid sequence comprising the VH of the antibody (e.g., the Light and/or Heavy Chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an anti-phosphorylated TDP-43 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of a TDP-43 antibody of the invention, nucleic acid encoding an antibody, e.g., as described above, is isolated and inserted into one or more vectors for further cloning and/or expression in a host cell or a cell-free expression system. Such nucleic acid may be readily isolated and sequenced using conventional procedures (e.g., by using oligonucleotide probes that are capable of binding specifically to genes encoding the Heavy and Light Chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Val. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CV1 line transformed by SV40 (COS-7); human embryonic kidney line (293 or 293 cells as described, e.g., in Graham et al., J. Gen Viral. 36:59 (1977)); baby hamster kidney cells (BHK); mouse Sertoli cells (TM4 cells as described, e.g., in Mather, Biol. Reprod. 23:243-251 (1980)); macaque kidney cells (CV 1); African green macaque kidney cells (VER0-76); human cervical carcinoma cells (HeLa); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N. Y Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Cii. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Val. 248 (B. K. C. Lo, ed., Humana Press, Totowa, NJ), pp. 255-268 (2003).

Several art-known approaches exist for delivering molecules across the blood brain barrier (BBB) such as alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gill et al., Nature Med. 9: 589-595 (2003); and Gliadel Wafers™, Guildford Pharmaceutical), and intranasal administration to bypass the BBB (Mittal et al, Drug Deliv. 21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

Trojan horse delivery methods of delivering the antibody or antibody fragment thereof across the blood brain barrier include, but are not limited to, cationizing the antibodies (see, e.g., U.S. Pat. No. 5,004,697), and the use of cell-penetration peptides such as Tat peptides to gain entry into the CNS. (see, e.g. Dietz et al., J. Neurochem. 104:757-765 (2008)).

Nanoparticle delivery methods of delivering the antibody or antigen-binding fragment thereof across the blood brain barrier include, but are not limited to, encapsulating the antibody or antigen-binding fragment thereof in liposomes, or extracellular vesicles such as exosomes, that are coupled to without limitation antibody or antigen-binding fragments or alternatively peptides that bind to receptors on the vascular endothelium of the blood-brain barrier (see, e.g., U.S. Patent Application Publication No. 20020025313), and coating the antibody or antigen-binding fragment thereof in low-density lipoprotein particles (see, e.g., U.S. Patent Application Publication No. 20040204354) or apolipoprotein E (see, e.g., U.S. Patent Application Publication No. 20040131692).

Antibodies of the invention can be further modified to enhance blood brain barrier penetration. The antibody or antigen-binding fragment thereof of the invention can be fused to a polypeptide binding to a blood-brain barrier receptor. BBB receptors include, but are not limited to, transferrin receptor, insulin receptor or low-density lipoprotein receptor. The polypeptide can be a peptide, a receptor ligand, a single domain antibody (VHH), a scFv or a Fab fragment.

Antibodies of the invention can also be delivered as a corresponding nucleic acid encoding for the antibody. Such nucleic acid molecule may be a part of a viral vector for targeted delivery to the blood brain barrier or any other cell type in the CNS. A non-limiting example is a viral vector comprising a nucleic acid molecule encoding an antibody of the invention for targeted delivery to endothelial cells of the BBB, pericytes of the BBB or astrocytes. In some embodiments the endothelial cells of the BBB, pericytes of the BBB or astrocytes express and secrete the antibody into the brain parenchyma. A viral vector may be a recombinant adeno-associated viral vector (rAAV) selected from any AAV serotype known in the art, including, without limitation, from AAV1 to AAV12 to enable the antibody or antibody fragment or antibody derivatives to be expressed intracellularly or into the brain parenchyma.

Cell therapy methods of delivering the antibody of the invention or antibody fragment or antibody derivatives across the blood brain barrier include, but are not limited to, the use of the homing capacity of Endothelial Progenitor Cells (EPCs) transfected ex vivo with vectors and the secretion and delivery of antibodies or antibody fragments to the brain by these cells, to overcome the powerful filtering activity of the Blood Brain Barrier (see, e.g., Heller and al., J Cell Mol Med. 00:1-7 (2020)), or the use of polymeric cell implant devices loaded with genetically engineered cells, to secrete antibody or antibody fragments (see, e.g. Marroquin Belaunzaran et al. PloS ONE 6(4): e18268 (2011)).

Pharmaceutically acceptable carriers, diluents, adjuvants and excipients are well known in the pharmaceutical art and are described, for example, in Remington's Pharmaceutical Sciences, 15^{th} or 18^{th} Ed. (Alfonso R. Gennaro, ed.; Mack Publishing Company, Easton, PA, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof.

The “effective amount” of the compound which is to be administered to a subject is the dosage which according to sound medical judgement is suitable for treating, preventing or alleviating the disease, disorder or abnormality. The specific dose level and frequency of dosage can depend, e.g., upon a variety of factors including the activity of the specific compound employed, the metabolic stability and length of action of that compound, mode and time of administration, the rate of excretion, and drug combination. Patient-specific factors such as the age, body weight, general health, sex, diet, as well as the severity of the particular condition can also influence the amount which is to be administered.

### INVENTION EMBODIMENTS FOR TDP-43 BINDING MOLECULE

In some embodiments, a TDP-43 binding molecule, in particular a TDP-43 antibody or antigen-binding fragment thereof is provided which comprises:
a) a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; or
b) a VH-CDR1 comprising the amino acid sequence of SEQ ID NO: 21; a VH-CDR2 comprising the amino acid sequence of SEQ ID NO: 22; and a VH-CDR3 comprising the amino acid sequence of SEQ ID NO: 23; or
c) a VH-CDR1 comprising the amino acid sequence of SEQ ID NO: 31; a VH-CDR2 comprising the amino acid sequence of SEQ ID NO: 32; and a VH-CDR3 comprising the amino acid sequence of SEQ ID NO: 33; or
d) a VH-CDR1 comprising the amino acid sequence of SEQ ID NO: 41; a VH-CDR2 comprising the amino acid sequence of SEQ ID NO: 42; and a VH-CDR3 comprising the amino acid sequence of SEQ ID NO: 33; or
e) a VH-CDR1 comprising the amino acid sequence of SEQ ID NO: 51; a VH-CDR2 comprising the amino acid sequence of SEQ ID NO: 52; and a VH-CDR3 comprising the amino acid sequence of SEQ ID NO: 53; or
f) a VH-CDR1 comprising the amino acid sequence of SEQ ID NO: 61; a VH-CDR2 comprising the amino acid sequence of SEQ ID NO: 62; and a VH-CDR3 comprising the amino acid sequence of SEQ ID NO: 63; or
g) a VH-CDR1 comprising the amino acid sequence of SEQ ID NO: 81; a VH-CDR2 comprising the amino acid sequence of SEQ ID NO: 82; and a VH-CDR3 comprising the amino acid sequence of SEQ ID NO: 83.

In some embodiments, a TDP-43 binding molecule, in particular a TDP-43 antibody or antigen-binding fragment thereof is provided which comprises:
a) a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; or
b) a VH-CDR1 comprising the amino acid sequence of SEQ ID NO: 21; a VH-CDR2 comprising the amino acid sequence of SEQ ID NO: 22; and a VH-CDR3 comprising the amino acid sequence of SEQ ID NO: 23; or
c) a VH-CDR1 comprising the amino acid sequence of SEQ ID NO: 31; a VH-CDR2 comprising the amino acid sequence of SEQ ID NO: 32; and a VH-CDR3 comprising the amino acid sequence of SEQ ID NO: 33; or
d) a VH-CDR1 comprising the amino acid sequence of SEQ ID NO: 41; a VH-CDR2 comprising the amino acid sequence of SEQ ID NO: 42; and a VH-CDR3 comprising the amino acid sequence of SEQ ID NO: 33; or
e) a VH-CDR1 comprising the amino acid sequence of SEQ ID NO: 51; a VH-CDR2 comprising the amino acid sequence of SEQ ID NO: 52; and a VH-CDR3 comprising the amino acid sequence of SEQ ID NO: 53; or
f) a VH-CDR1 comprising the amino acid sequence of SEQ ID NO: 61; a VH-CDR2 comprising the amino acid sequence of SEQ ID NO: 62; and a VH-CDR3 comprising the amino acid sequence of SEQ ID NO: 63; or
g) a VH-CDR1 comprising the amino acid sequence of SEQ ID NO: 81; a VH-CDR2 comprising the amino acid sequence of SEQ ID NO: 82; and a VH-CDR3 comprising the amino acid sequence of SEQ ID NO: 83; or
h) a VH-CDR1 comprising the amino acid sequence of SEQ ID NO: 111; a VH-CDR2 comprising the amino acid sequence of SEQ ID NO: 112; and a VH-CDR3 comprising the amino acid sequence of SEQ ID NO: 113; or

In some embodiments, a TDP-43 binding molecule, in particular a TDP-43 antibody or antigen-binding fragment thereof is provided which comprises:
a) a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VL-CDR1 comprising the amino acid sequence of SEQ ID NO: 25; a VL-CDR2 comprising the amino acid sequence of SEQ ID NO: 26; and a VL-CDR3 comprising the amino acid sequence of SEQ ID NO: 27; or
c) a VL-CDR1 comprising the amino acid sequence of SEQ ID NO: 35; a VL-CDR2 comprising the amino acid sequence of SEQ ID NO: 16; and a VL-CDR3 comprising the amino acid sequence of SEQ ID NO: 37; or
d) a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
e) a VL-CDR1 comprising the amino acid sequence of SEQ ID NO: 55; a VL-CDR2 comprising the amino acid sequence of SEQ ID NO: 26; and a VL-CDR3 comprising the amino acid sequence of SEQ ID NO: 57; or
f) a VL-CDR1 comprising the amino acid sequence of SEQ ID NO: 75; a VL-CDR2 comprising the amino acid sequence of SEQ ID NO: 76; and a VL-CDR3 comprising the amino acid sequence of SEQ ID NO: 77; or
g) a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87.

In some embodiments, a TDP-43 binding molecule, in particular a TDP-43 antibody or antigen-binding fragment thereof is provided which comprises:
a) a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VL-CDR1 comprising the amino acid sequence of SEQ ID NO: 25; a VL-CDR2 comprising the amino acid sequence of SEQ ID NO: 26; and a VL-CDR3 comprising the amino acid sequence of SEQ ID NO: 27; or
c) a VL-CDR1 comprising the amino acid sequence of SEQ ID NO: 35; a VL-CDR2 comprising the amino acid sequence of SEQ ID NO: 16; and a VL-CDR3 comprising the amino acid sequence of SEQ ID NO: 37; or
d) a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
e) a VL-CDR1 comprising the amino acid sequence of SEQ ID NO: 55; a VL-CDR2 comprising the amino acid sequence of SEQ ID NO: 26; and a VL-CDR3 comprising the amino acid sequence of SEQ ID NO: 57; or
f) a VL-CDR1 comprising the amino acid sequence of SEQ ID NO: 75; a VL-CDR2 comprising the amino acid sequence of SEQ ID NO: 76; and a VL-CDR3 comprising the amino acid sequence of SEQ ID NO: 77; or
g) a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
h) a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117.

In some embodiments, a TDP-43 binding molecule, in particular a TDP-43 antibody or antigen-binding fragment thereof is provided which comprises:
a) a Heavy Chain Variable Region (VH) comprising:
   i. a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; or
   ii. a VH-CDR1 comprising the amino acid sequence of SEQ ID NO: 21; a VH-CDR2 comprising the amino acid sequence of SEQ ID NO: 22; and a VH-CDR3 comprising the amino acid sequence of SEQ ID NO: 23; or
   iii. a VH-CDR1 comprising the amino acid sequence of SEQ ID NO: 31; a VH-CDR2 comprising the amino acid sequence of SEQ ID NO: 32; and a VH-CDR3 comprising the amino acid sequence of SEQ ID NO: 33; or
   iv. a VH-CDR1 comprising the amino acid sequence of SEQ ID NO: 41; a VH-CDR2 comprising the amino acid sequence of SEQ ID NO: 42; and a VH-CDR3 comprising the amino acid sequence of SEQ ID NO: 33; or
   v. a VH-CDR1 comprising the amino acid sequence of SEQ ID NO: 51; a VH-CDR2 comprising the amino acid sequence of SEQ ID NO: 52; and a VH-CDR3 comprising the amino acid sequence of SEQ ID NO: 53; or
   vi. a VH-CDR1 comprising the amino acid sequence of SEQ ID NO: 61; a VH-CDR2 comprising the amino acid sequence of SEQ ID NO: 62; and a VH-CDR3 comprising the amino acid sequence of SEQ ID NO: 63; or
   vii. a VH-CDR1 comprising the amino acid sequence of SEQ ID NO: 81; a VH-CDR2 comprising the amino acid sequence of SEQ ID NO: 82; and a VH-CDR3 comprising the amino acid sequence of SEQ ID NO: 83; and
b) a Light Chain Variable Region (VL) comprising:
   i. a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
   ii. a VL-CDR1 comprising the amino acid sequence of SEQ ID NO: 25; a VL-CDR2 comprising the amino acid sequence of SEQ ID NO: 26; and a VL-CDR3 comprising the amino acid sequence of SEQ ID NO: 27; or
   iii. a VL-CDR1 comprising the amino acid sequence of SEQ ID NO: 35; a VL-CDR2 comprising the amino acid sequence of SEQ ID NO: 16; and a VL-CDR3 comprising the amino acid sequence of SEQ ID NO: 37; or
   iv. a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
   v. a VL-CDR1 comprising the amino acid sequence of SEQ ID NO: 55; a VL-CDR2 comprising the amino acid sequence of SEQ ID NO: 26; and a VL-CDR3 comprising the amino acid sequence of SEQ ID NO: 57; or
   vi. a VL-CDR1 comprising the amino acid sequence of SEQ ID NO: 75; a VL-CDR2 comprising the amino acid sequence of SEQ ID NO: 76; and a VL-CDR3 comprising the amino acid sequence of SEQ ID NO: 77; or
   vii. a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87.

In some embodiments, a TDP-43 binding molecule, in particular a TDP-43 antibody or antigen-binding fragment thereof is provided which comprises:
a) a Heavy Chain Variable Region (VH) comprising:
   i. a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; or
   ii. a VH-CDR1 comprising the amino acid sequence of SEQ ID NO: 21; a VH-CDR2 comprising the amino acid sequence of SEQ ID NO: 22; and a VH-CDR3 comprising the amino acid sequence of SEQ ID NO: 23; or
   iii. a VH-CDR1 comprising the amino acid sequence of SEQ ID NO: 31; a VH-CDR2 comprising the amino acid sequence of SEQ ID NO: 32; and a VH-CDR3 comprising the amino acid sequence of SEQ ID NO: 33; or
   iv. a VH-CDR1 comprising the amino acid sequence of SEQ ID NO: 41; a VH-CDR2 comprising the amino acid sequence of SEQ ID NO: 42; and a VH-CDR3 comprising the amino acid sequence of SEQ ID NO: 33; or
   v. a VH-CDR1 comprising the amino acid sequence of SEQ ID NO: 51; a VH-CDR2 comprising the amino acid sequence of SEQ ID NO: 52; and a VH-CDR3 comprising the amino acid sequence of SEQ ID NO: 53; or
   vi. a VH-CDR1 comprising the amino acid sequence of SEQ ID NO: 61; a VH-CDR2 comprising the amino acid sequence of SEQ ID NO: 62; and a VH-CDR3 comprising the amino acid sequence of SEQ ID NO: 63; or
   vii. a VH-CDR1 comprising the amino acid sequence of SEQ ID NO: 81; a VH-CDR2 comprising the amino acid sequence of SEQ ID NO: 82; and a VH-CDR3 comprising the amino acid sequence of SEQ ID NO: 83; or
   viii. a VH-CDR1 comprising the amino acid sequence of SEQ ID NO: 111; a VH-CDR2 comprising the amino acid sequence of SEQ ID NO: 112; and a VH-CDR3 comprising the amino acid sequence of SEQ ID NO: 113; and
b) a Light Chain Variable Region (VL) comprising:
   i. a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
   ii. a VL-CDR1 comprising the amino acid sequence of SEQ ID NO: 25; a VL-CDR2 comprising the amino acid sequence of SEQ ID NO: 26; and a VL-CDR3 comprising the amino acid sequence of SEQ ID NO: 27; or
   iii. a VL-CDR1 comprising the amino acid sequence of SEQ ID NO: 35; a VL-CDR2 comprising the amino acid sequence of SEQ ID NO: 16; and a VL-CDR3 comprising the amino acid sequence of SEQ ID NO: 37; or
   iv. a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
   v. a VL-CDR1 comprising the amino acid sequence of SEQ ID NO: 55; a VL-CDR2 comprising the amino acid sequence of SEQ ID NO: 26; and a VL-CDR3 comprising the amino acid sequence of SEQ ID NO: 57; or
   vi. a VL-CDR1 comprising the amino acid sequence of SEQ ID NO: 75; a VL-CDR2 comprising the amino acid sequence of SEQ ID NO: 76; and a VL-CDR3 comprising the amino acid sequence of SEQ ID NO: 77; or
   vii. a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
   viii. a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117.

In some embodiments, a TDP-43 binding molecule, in particular a TDP-43 antibody or antigen-fragment thereof is provided which comprises:
a) a Heavy Chain Variable Region (VH) comprising:
   i. a VH-CDR1 comprising the amino acid sequence of SEQ ID NO: 11 or a VH-CDR1 comprising an amino acid sequence having at least 80%, 90%, 95% or 100% sequence identity to SEQ ID NO: 11; a VH-CDR2 comprising the amino acid sequence of SEQ ID NO: 12 or a VH-CDR2 comprising an amino acid sequence having at least 80%, 90%, 95% or 100% sequence identity to SEQ ID NO: 12; and a VH-CDR3 comprising the amino acid sequence of SEQ ID NO: 13 or a VH-CDR3 comprising an amino acid sequence having at least 80%, 90%, 95% or 100% sequence identity to SEQ ID NO: 13; or
   ii. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence of SEQ ID NO: 23 or a VH-CDR3 comprising an amino acid sequence having at least 80%, 90%, 95% or 100% sequence identity to SEQ ID NO: 23; or
   iii. a VH-CDR1 comprising the amino acid sequence of SEQ ID NO: 31 or a VH-CDR1 comprising an amino acid sequence having at least 80%, 90%, 95% or 100% sequence identity to SEQ ID NO: 31; a VH-CDR2 comprising the amino acid sequence of SEQ ID NO: 32 or a VH-CDR2 comprising an amino acid sequence having at least 80%, 90%, 95% or 100% sequence identity to SEQ ID NO: 32; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; or
   iv. a VH-CDR1 comprising the amino acid sequence of SEQ ID NO: 41 or a VH-CDR1 comprising an amino acid sequence having at least 80%, 90%, 95% or 100% sequence identity to SEQ ID NO: 41; a VH-CDR2 comprising the amino acid sequence of SEQ ID NO: 42 or a VH-CDR2 comprising an amino acid sequence having at least 80%, 90%, 95% or 100% sequence identity to SEQ ID NO: 42; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; or
   v. a VH-CDR1 comprising the amino acid sequence of SEQ ID NO: 51 or a VH-CDR1 comprising an amino acid sequence having at least 80%, 90%, 95% or 100% sequence identity to SEQ ID NO: 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; or
   vi. a VH-CDR1 comprising the amino acid sequence of SEQ ID NO: 61 or a VH-CDR1 comprising an amino acid sequence having at least 80%, 90%, 95% or 100% sequence identity to SEQ ID NO: 61; a VH-CDR2 comprising the amino acid sequence of SEQ ID NO: 62 or a VH-CDR2 comprising an amino acid sequence having at least 80%, 90%, 95% or 100% sequence identity to SEQ ID NO: 62; and a VH-CDR3 comprising the amino acid sequence of SEQ ID NO: 63 or a VH-CDR3 comprising an amino acid sequence having at least 80%, 90%, 95% or 100% sequence identity to SEQ ID NO: 63; or
   vii. a VH-CDR1 comprising the amino acid sequence of SEQ ID NO: 81 or a VH-CDR1 comprising an amino acid sequence having at least 80%, 90%, 95% or 100% sequence identity to SEQ ID NO: 81; a VH-CDR2 comprising the amino acid sequence of SEQ ID NO: 82 or a VH-CDR2 comprising an amino acid sequence having at least 80%, 90%, 95% or 100% sequence identity to SEQ ID NO: 82; and a VH-CDR3 comprising the amino acid sequence of SEQ ID NO: 83 or a VH-CDR3 comprising an amino acid sequence having at least 80%, 90%, 95% or 100% sequence identity to SEQ ID NO: 83; and
b) a Light Chain Variable Region (VL) comprising:
   i. a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17; or
   ii. a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
   iii. a VL-CDR1 comprising the amino acid sequence of SEQ ID NO: 35 or a VL-CDR1 comprising an amino acid sequence having at least 80%, 90%, 95% or 100% sequence identity to SEQ ID NO: 35; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 37 or a VL-CDR3 comprising an amino acid sequence having at least 80%, 90%, 95% or 100% sequence identity to SEQ ID NO: 37; or
   iv. a VL-CDR1 comprising the amino acid sequence of SEQ ID NO: 45 or a VL-CDR1 comprising an amino acid sequence having at least 80%, 90%, 95% or 100% sequence identity to SEQ ID NO: 45; a VL-CDR2 comprising the amino acid sequence of SEQ ID NO: 46 or a VL-CDR2 comprising an amino acid sequence having at least 80%, 90%, 95% or 100% sequence identity to SEQ ID NO: 46; and a VL-CDR3 comprising the amino acid sequence of SEQ ID NO: 47 or a VL-CDR3 comprising an amino acid sequence having at least 80%, 90%, 95% or 100% sequence identity to SEQ ID NO: 47; or
   v. a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
   vi. a VL-CDR1 comprising the amino acid sequence of SEQ ID NO: 75 or a VL-CDR1 comprising an amino acid sequence having at least 80%, 90%, 95% or 100% sequence identity to SEQ ID NO: 75; a VL-CDR2 comprising the amino acid sequence of SEQ ID NO: 76 or a VL-CDR2 comprising an amino acid sequence having at least 80%, 90%, 95% or 100% sequence identity to SEQ ID NO: 76; and a VL-CDR3 comprising the amino acid sequence of SEQ ID NO: 77 or a VL-CDR3 comprising an amino acid sequence having at least 80%, 90%, 95% or 100% sequence identity to SEQ ID NO: 77; or
   vii. a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

In some embodiments, a TDP-43 binding molecule, in particular a TDP-43 antibody or antigen-binding fragment thereof is provided which comprises:
a) a Heavy Chain Variable Region (VH) comprising:
   i. a VH-CDR1 comprising the amino acid sequence of SEQ ID NO: 11 or a VH-CDR1 comprising an amino acid sequence having at least 80%, 90%, 95% or 100% sequence identity to SEQ ID NO: 11; a VH-CDR2 comprising the amino acid sequence of SEQ ID NO: 12 or a VH-CDR2 comprising an amino acid sequence having at least 80%, 90%, 95% or 100% sequence identity to SEQ ID NO: 12; and a VH-CDR3 comprising the amino acid sequence of SEQ ID NO: 13 or a VH-CDR3 comprising an amino acid sequence having at least 80%, 90%, 95% or 100% sequence identity to SEQ ID NO: 13; or
   ii. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence of SEQ ID NO: 23 or a VH-CDR3 comprising an amino acid sequence having at least 80%, 90%, 95% or 100% sequence identity to SEQ ID NO: 23; or
   iii. a VH-CDR1 comprising the amino acid sequence of SEQ ID NO: 31 or a VH-CDR1 comprising an amino acid sequence having at least 80%, 90%, 95% or 100% sequence identity to SEQ ID NO: 31; a VH-CDR2 comprising the amino acid sequence of SEQ ID NO: 32 or a VH-CDR2 comprising an amino acid sequence having at least 80%, 90%, 95% or 100% sequence identity to SEQ ID NO: 32; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; or
   iv. a VH-CDR1 comprising the amino acid sequence of SEQ ID NO: 41 or a VH-CDR1 comprising an amino acid sequence having at least 80%, 90%, 95% or 100% sequence identity to SEQ ID NO: 41; a VH-CDR2 comprising the amino acid sequence of SEQ ID NO: 42 or a VH-CDR2 comprising an amino acid sequence having at least 80%, 90%, 95% or 100% sequence identity to SEQ ID NO: 42; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; or
   v. a VH-CDR1 comprising the amino acid sequence of SEQ ID NO: 51 or a VH-CDR1 comprising an amino acid sequence having at least 80%, 90%, 95% or 100% sequence identity to SEQ ID NO: 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; or
   vi. a VH-CDR1 comprising the amino acid sequence of SEQ ID NO: 61 or a VH-CDR1 comprising an amino acid sequence having at least 80%, 90%, 95% or 100% sequence identity to SEQ ID NO: 61; a VH-CDR2 comprising the amino acid sequence of SEQ ID NO: 62 or a VH-CDR2 comprising an amino acid sequence having at least 80%, 90%, 95% or 100% sequence identity to SEQ ID NO: 62; and a VH-CDR3 comprising the amino acid sequence of SEQ ID NO: 63 or a VH-CDR3 comprising an amino acid sequence having at least 80%, 90%, 95% or 100% sequence identity to SEQ ID NO: 63; or
   vii. a VH-CDR1 comprising the amino acid sequence of SEQ ID NO: 81 or a VH-CDR1 comprising an amino acid sequence having at least 80%, 90%, 95% or 100% sequence identity to SEQ ID NO: 81; a VH-CDR2 comprising the amino acid sequence of SEQ ID NO: 82 or a VH-CDR2 comprising an amino acid sequence having at least 80%, 90%, 95% or 100% sequence identity to SEQ ID NO: 82; and a VH-CDR3 comprising the amino acid sequence of SEQ ID NO: 83 or a VH-CDR3 comprising an amino acid sequence having at least 80%, 90%, 95% or 100% sequence identity to SEQ ID NO: 83; or
   viii. a VH-CDR1 comprising the amino acid sequence of SEQ ID NO: 111 or a VH-CDR1 comprising an amino acid sequence having at least 80%, 90%, 95% or 100% sequence identity to SEQ ID NO: 111; a VH-CDR2 comprising the amino acid sequence of SEQ ID NO: 112 or a VH-CDR2 comprising an amino acid sequence having at least 80%, 90%, 95% or 100% sequence identity to SEQ ID NO: 112; and a VH-CDR3 comprising the amino acid sequence of SEQ ID NO: 113 or a VH-CDR3 comprising an amino acid sequence having at least 80%, 90%, 95% or 100% sequence identity to SEQ ID NO: 113; and
b) a Light Chain Variable Region (VL) comprising:
   i. a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17; or
   ii. a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
   iii. a VL-CDR1 comprising the amino acid sequence of SEQ ID NO: 35 or a VL-CDR1 comprising an amino acid sequence having at least 80%, 90%, 95% or 100% sequence identity to SEQ ID NO: 35; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 37 or a VL-CDR3 comprising an amino acid sequence having at least 80%, 90%, 95% or 100% sequence identity to SEQ ID NO: 37; or
   iv. a VL-CDR1 comprising the amino acid sequence of SEQ ID NO: 45 or a VL-CDR1 comprising an amino acid sequence having at least 80%, 90%, 95% or 100% sequence identity to SEQ ID NO: 45; a VL-CDR2 comprising the amino acid sequence of SEQ ID NO: 46 or a VL-CDR2 comprising an amino acid sequence having at least 80%, 90%, 95% or 100% sequence identity to SEQ ID NO: 46; and a VL-CDR3 comprising the amino acid sequence of SEQ ID NO: 47 or a VL-CDR3 comprising an amino acid sequence having at least 80%, 90%, 95% or 100% sequence identity to SEQ ID NO: 47; or
   v. a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
   vi. a VL-CDR1 comprising the amino acid sequence of SEQ ID NO: 75 or a VL-CDR1 comprising an amino acid sequence having at least 80%, 90%, 95% or 100% sequence identity to SEQ ID NO: 75; a VL-CDR2 comprising the amino acid sequence of SEQ ID NO: 76 or a VL-CDR2 comprising an amino acid sequence having at least 80%, 90%, 95% or 100% sequence identity to SEQ ID NO: 76; and a VL-CDR3 comprising the amino acid sequence of SEQ ID NO: 77 or a VL-CDR3 comprising an amino acid sequence having at least 80%, 90%, 95% or 100% sequence identity to SEQ ID NO: 77; or
   vii. a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87; or
   viii. a VL-CDR1 comprising the amino acid sequence of SEQ ID NO: 115 or a VL-CDR1 comprising an amino acid sequence having at least 80%, 90%, 95% or 100% sequence identity to SEQ ID NO: 115; a VL-CDR2 comprising the amino acid sequence of SEQ ID NO: 116 or a VL-CDR2 comprising an amino acid sequence having at least 80%, 90%, 95% or 100% sequence identity to SEQ ID NO: 116; and a VL-CDR3 comprising the amino acid sequence of SEQ ID NO: 117 or a VL-CDR3 comprising an amino acid sequence having at least 80%, 90%, 95% or 100% sequence identity to SEQ ID NO: 117.

Similarly, in some embodiments, a TDP-43 binding molecule, in particular a TDP-43 antibody or antigen-binding fragment thereof is provided which comprises:
a) a Heavy Chain Variable Region (VH) comprising a VH-CDR1 comprising the amino acid sequence of SEQ ID NO: 11 or a VH-CDR1 comprising an amino acid sequence having at least 80%, 90%, 95% or 100% sequence identity to SEQ ID NO 11; a VH-CDR2 comprising the amino acid sequence of SEQ ID NO: 12 or a VH-CDR2 comprising an amino acid sequence having at least 80%, 90%, 95% or 100% sequence identity to SEQ ID NO 12; and a VH-CDR3 comprising the amino acid sequence of SEQ ID NO: 13 or a VH-CDR3 comprising an amino acid sequence having at least 80%, 90%, 95% or 100% sequence identity to SEQ ID NO: 13; and a Light Chain Variable Region (VL) comprising a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17; or
b) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence of SEQ ID NO: 23 or a VH-CDR3 comprising an amino acid sequence having at least 80%, 90%, 95% or 100% sequence identity to SEQ ID NO 23; and a Light Chain Variable Region (VL) comprising a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
c) a Heavy Chain Variable Region (VH) comprising a VH-CDR1 comprising the amino acid sequence of SEQ ID NO: 31 or a VH-CDR1 comprising an amino acid sequence having at least 80%, 90%, 95% or 100% sequence identity to SEQ ID NO 31; a VH-CDR2 comprising the amino acid sequence of SEQ ID NO: 32 or a VH-CDR2 comprising an amino acid sequence having at least 80%, 90%, 95% or 100% sequence identity to SEQ ID NO: 32; and a VH-CDR3 comprising the amino acid sequence of SEQ ID NO: 33 or a VH-CDR3 comprising an amino acid sequence having at least 80%, 90%, 95% or 100% sequence identity to SEQ ID NO 33; and a Light Chain Variable Region (VL) comprising a VL-CDR1 comprising the amino acid sequence of SEQ ID NO: 35 or a VL-CDR1 comprising an amino acid sequence having at least 80%, 90%, 95% or 100% sequence identity to SEQ ID NO: 35; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 37 or a VL-CDR3 comprising an amino acid sequence having at least 80%, 90%, 95% or 100% sequence identity to SEQ ID NO: 37; or
d) a Heavy Chain Variable Region (VH) comprising a VH-CDR1 comprising the amino acid sequence of SEQ ID NO: 41 or a VH-CDR1 comprising an amino acid sequence having at least 80%, 90%, 95% or 100% sequence identity to SEQ ID NO 41; a VH-CDR2 comprising the amino acid sequence of SEQ ID NO: 42 or a VH-CDR2 comprising an amino acid sequence having at least 80%, 90%, 95% or 100% sequence identity to SEQ ID NO: 42; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; and a Light Chain Variable Region (VL) comprising a VL-CDR1 comprising the amino acid sequence of SEQ ID NO: 45 or a VL-CDR1 comprising an amino acid sequence having at least 80%, 90%, 95% or 100% sequence identity to SEQ ID NO: 45; a VL-CDR2 comprising the amino acid sequence of SEQ ID NO: 46 or a VL-CDR2 comprising an amino acid sequence having at least 80%, 90%, 95% or 100% sequence identity to SEQ ID NO: 46; and a VL-CDR3 comprising the amino acid sequence of SEQ ID NO: 47 or a VL-CDR3 comprising an amino acid sequence having at least 80%, 90%, 95% or 100% sequence identity to SEQ ID NO: 47; or
e) a Heavy Chain Variable Region (VH) comprising a VH-CDR1 comprising the amino acid sequence of SEQ ID NO: 51 or a VH-CDR1 comprising an amino acid sequence having at least 80%, 90%, 95% or 100% sequence identity to SEQ ID NO 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; and a Light Chain Variable Region (VL) comprising a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
f) a Heavy Chain Variable Region (VH) comprising a VH-CDR1 comprising the amino acid sequence of SEQ ID NO: 61 or a VH-CDR1 comprising an amino acid sequence having at least 80%, 90%, 95% or 100% sequence identity to SEQ ID NO 61; a VH-CDR2 comprising the amino acid sequence of SEQ ID NO: 62 or a VH-CDR2 comprising an amino acid sequence having at least 80%, 90%, 95% or 100% sequence identity to SEQ ID NO: 62; and a VH-CDR3 comprising the amino acid sequence of SEQ ID NO: 63 or a VH-CDR3 comprising an amino acid sequence having at least 80%, 90%, 95% or 100% sequence identity to SEQ ID NO: 63; and a Light Chain Variable Region (VL) comprising a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
g) a Heavy Chain Variable Region (VH) comprising a VH-CDR1 comprising the amino acid sequence of SEQ ID NO: 51 or a VH-CDR1 comprising an amino acid sequence having at least 80%, 90%, 95% or 100% sequence identity to SEQ ID NO 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; and a Light Chain Variable Region (VL) comprising a VL-CDR1 comprising the amino acid sequence of SEQ ID NO: 75 or a VL-CDR1 comprising an amino acid sequence having at least 80%, 90%, 95% or 100% sequence identity to SEQ ID NO: 75; a VL-CDR2 comprising the amino acid sequence of SEQ ID NO: 76 or a VL-CDR2 comprising an amino acid sequence having at least 80%, 90%, 95% or 100% sequence identity to SEQ ID NO: 76; and a VL-CDR3 comprising the amino acid sequence of SEQ ID NO: 77 or a VL-CDR3 comprising an amino acid sequence having at least 80%, 90%, 95% or 100% sequence identity to SEQ ID NO: 77; or
h) a Heavy Chain Variable Region (VH) comprising a VH-CDR1 comprising the amino acid sequence of SEQ ID NO: 81 or a VH-CDR1 comprising an amino acid sequence having at least 80%, 90%, 95% or 100% sequence identity to SEQ ID NO 81; a VH-CDR2 comprising the amino acid sequence of SEQ ID NO: 82 or a VH-CDR2 comprising an amino acid sequence having at least 80%, 90%, 95% or 100% sequence identity to SEQ ID NO: 82; and a VH-CDR3 comprising the amino acid sequence of SEQ ID NO: 83 or a VH-CDR3 comprising an amino acid sequence having at least 80%, 90%, 95% or 100% sequence identity to SEQ ID NO: 83; and a Light Chain Variable Region (VL) comprising a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

Similarly, in some embodiments, a TDP-43 binding molecule, in particular a TDP-43 antibody or antigen-binding fragment thereof is provided which comprises:
a) a Heavy Chain Variable Region (VH) comprising a VH-CDR1 comprising the amino acid sequence of SEQ ID NO: 11 or a VH-CDR1 comprising an amino acid sequence having at least 80%, 90%, 95% or 100% sequence identity to SEQ ID NO 11; a VH-CDR2 comprising the amino acid sequence of SEQ ID NO: 12 or a VH-CDR2 comprising an amino acid sequence having at least 80%, 90%, 95% or 100% sequence identity to SEQ ID NO 12; and a VH-CDR3 comprising the amino acid sequence of SEQ ID NO: 13 or a VH-CDR3 comprising an amino acid sequence having at least 80%, 90%, 95% or 100% sequence identity to SEQ ID NO: 13; and a Light Chain Variable Region (VL) comprising a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17; or
b) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence of SEQ ID NO: 23 or a VH-CDR3 comprising an amino acid sequence having at least 80%, 90%, 95% or 100% sequence identity to SEQ ID NO 23; and a Light Chain Variable Region (VL) comprising a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
c) a Heavy Chain Variable Region (VH) comprising a VH-CDR1 comprising the amino acid sequence of SEQ ID NO: 31 or a VH-CDR1 comprising an amino acid sequence having at least 80%, 90%, 95% or 100% sequence identity to SEQ ID NO 31; a VH-CDR2 comprising the amino acid sequence of SEQ ID NO: 32 or a VH-CDR2 comprising an amino acid sequence having at least 80%, 90%, 95% or 100% sequence identity to SEQ ID NO: 32; and a VH-CDR3 comprising the amino acid sequence of SEQ ID NO: 33 or a VH-CDR3 comprising an amino acid sequence having at least 80%, 90%, 95% or 100% sequence identity to SEQ ID NO 33; and a Light Chain Variable Region (VL) comprising a VL-CDR1 comprising the amino acid sequence of SEQ ID NO: 35 or a VL-CDR1 comprising an amino acid sequence having at least 80%, 90%, 95% or 100% sequence identity to SEQ ID NO: 35; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 37 or a VL-CDR3 comprising an amino acid sequence having at least 80%, 90%, 95% or 100% sequence identity to SEQ ID NO: 37; or
d) a Heavy Chain Variable Region (VH) comprising a VH-CDR1 comprising the amino acid sequence of SEQ ID NO: 41 or a VH-CDR1 comprising an amino acid sequence having at least 80%, 90%, 95% or 100% sequence identity to SEQ ID NO 41; a VH-CDR2 comprising the amino acid sequence of SEQ ID NO: 42 or a VH-CDR2 comprising an amino acid sequence having at least 80%, 90%, 95% or 100% sequence identity to SEQ ID NO: 42; and a VH-CDR3 comprising the amino acid sequence of SEQ ID NO: 33 or a VH-CDR3 comprising an amino acid sequence having at least 80%, 90%, 95% or 100% sequence identity to SEQ ID NO: 33; and a Light Chain Variable Region (VL) comprising a VL-CDR1 comprising the amino acid sequence of SEQ ID NO: 45 or a VL-CDR1 comprising an amino acid sequence having at least 80%, 90%, 95% or 100% sequence identity to SEQ ID NO: 45; a VL-CDR2 comprising the amino acid sequence of SEQ ID NO: 46 or a VL-CDR2 comprising an amino acid sequence having at least 80%, 90%, 95% or 100% sequence identity to SEQ ID NO: 46; and a VL-CDR3 comprising the amino acid sequence of SEQ ID NO: 47 or a VL-CDR3 comprising an amino acid sequence having at least 80%, 90%, 95% or 100% sequence identity to SEQ ID NO: 47; or
e) a Heavy Chain Variable Region (VH) comprising a VH-CDR1 comprising the amino acid sequence of SEQ ID NO: 51 or a VH-CDR1 comprising an amino acid sequence having at least 80%, 90%, 95% or 100% sequence identity to SEQ ID NO 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; and a Light Chain Variable Region (VL) comprising a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
f) a Heavy Chain Variable Region (VH) comprising a VH-CDR1 comprising the amino acid sequence of SEQ ID NO: 61 or a VH-CDR1 comprising an amino acid sequence having at least 80%, 90%, 95% or 100% sequence identity to SEQ ID NO 61; a VH-CDR2 comprising the amino acid sequence of SEQ ID NO: 62 or a VH-CDR2 comprising an amino acid sequence having at least 80%, 90%, 95% or 100% sequence identity to SEQ ID NO: 62; and a VH-CDR3 comprising the amino acid sequence of SEQ ID NO: 63 or a VH-CDR3 comprising an amino acid sequence having at least 80%, 90%, 95% or 100% sequence identity to SEQ ID NO: 63; and a Light Chain Variable Region (VL) comprising a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
g) a Heavy Chain Variable Region (VH) comprising a VH-CDR1 comprising the amino acid sequence of SEQ ID NO: 51 or a VH-CDR1 comprising an amino acid sequence having at least 80%, 90%, 95% or 100% sequence identity to SEQ ID NO 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; and a Light Chain Variable Region (VL) comprising a VL-CDR1 comprising the amino acid sequence of SEQ ID NO: 75 or a VL-CDR1 comprising an amino acid sequence having at least 80%, 90%, 95% or 100% sequence identity to SEQ ID NO: 75; a VL-CDR2 comprising the amino acid sequence of SEQ ID NO: 76 or a VL-CDR2 comprising an amino acid sequence having at least 80%, 90%, 95% or 100% sequence identity to SEQ ID NO: 76; and a VL-CDR3 comprising the amino acid sequence of SEQ ID NO: 77 or a VL-CDR3 comprising an amino acid sequence having at least 80%, 90%, 95% or 100% sequence identity to SEQ ID NO: 77; or
h) a Heavy Chain Variable Region (VH) comprising a VH-CDR1 comprising the amino acid sequence of SEQ ID NO: 81 or a VH-CDR1 comprising an amino acid sequence having at least 80%, 90%, 95% or 100% sequence identity to SEQ ID NO 81; a VH-CDR2 comprising the amino acid sequence of SEQ ID NO: 82 or a VH-CDR2 comprising an amino acid sequence having at least 80%, 90%, 95% or 100% sequence identity to SEQ ID NO: 82; and a VH-CDR3 comprising the amino acid sequence of SEQ ID NO: 83 or a VH-CDR3 comprising an amino acid sequence having at least 80%, 90%, 95% or 100% sequence identity to SEQ ID NO: 83; and a Light Chain Variable Region (VL) comprising a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87; or
i) Heavy Chain Variable Region (VH) comprising a VH-CDR1 comprising the amino acid sequence of SEQ ID NO: 111 or a VH-CDR1 comprising an amino acid sequence having at least 80%, 90%, 95% or 100% sequence identity to SEQ ID NO 111; a VH-CDR2 comprising the amino acid sequence of SEQ ID NO: 112 or a VH-CDR2 comprising an amino acid sequence having at least 80%, 90%, 95% or 100% sequence identity to SEQ ID NO: 112; and a VH-CDR3 comprising the amino acid sequence of SEQ ID NO: 113 or a VH-CDR3 comprising an amino acid sequence having at least 80%, 90%, 95% or 100% sequence identity to SEQ ID NO: 113; and a Light Chain Variable Region (VL) comprising a VL-CDR1 comprising the amino acid sequence of SEQ ID NO: 115 or a VL-CDR1 comprising an amino acid sequence having at least 80%, 90%, 95% or 100% sequence identity to SEQ ID NO: 115; a VL-CDR2 comprising the amino acid sequence of SEQ ID NO: 116 or a VL-CDR2 comprising an amino acid sequence having at least 80%, 90%, 95% or 100% sequence identity to SEQ ID NO: 116; and a VL-CDR3 comprising the amino acid sequence of SEQ ID NO: 117 or a VL-CDR3 comprising an amino acid sequence having at least 80%, 90%, 95% or 100% sequence identity to SEQ ID NO: 117.

Similarly, in some embodiments, a TDP-43 binding molecule, in particular a TDP-43 antibody or antigen-binding fragment thereof is provided which comprises:
a) a Heavy Chain Variable Region (VH) comprising a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and a Light Chain Variable Region (VL) comprising a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
b) a Heavy Chain Variable Region (VH) comprising a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; and a Light Chain Variable Region (VL) comprising a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
c) a Heavy Chain Variable Region (VH) comprising a VH-CDR1 comprising the amino acid sequence of SEQ ID NO: 31, a VH-CDR2 comprising the amino acid sequence of SEQ ID NO: 32 and a VH-CDR3 comprising the amino acid sequence of SEQ ID NO: 33; and a Light Chain Variable Region (VL) comprising a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
d) a Heavy Chain Variable Region (VH) comprising a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 33; and a Light Chain Variable Region (VL) comprising a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
e) a Heavy Chain Variable Region (VH) comprising a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; and a Light Chain Variable Region (VL) comprising a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
f) a Heavy Chain Variable Region (VH) comprising a VH-CDR1 comprising the amino acid sequence of SEQ ID NO: 61, a VH-CDR2 comprising the amino acid sequence of SEQ ID NO: 62 and a VH-CDR3 comprising the amino acid sequence of SEQ ID NO: 63; and a Light Chain Variable Region (VL) comprising a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
g) a Heavy Chain Variable Region (VH) comprising a VH-CDR1 comprising the amino acid sequence of SEQ ID NO: 51, a VH-CDR2 comprising the amino acid sequence of SEQ ID NO: 52, and a VH-CDR3 comprising the amino acid sequence of SEQ ID NO: 53; and a Light Chain Variable Region (VL) comprising a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
h) a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.

Similarly, in some embodiments, a TDP-43 binding molecule, in particular a TDP-43 antibody or antigen-binding fragment thereof is provided which comprises:
a) a Heavy Chain Variable Region (VH) comprising a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and a Light Chain Variable Region (VL) comprising a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
b) a Heavy Chain Variable Region (VH) comprising a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; and a Light Chain Variable Region (VL) comprising a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
c) a Heavy Chain Variable Region (VH) comprising a VH-CDR1 comprising the amino acid sequence of SEQ ID NO: 31, a VH-CDR2 comprising the amino acid sequence of SEQ ID NO: 32 and a VH-CDR3 comprising the amino acid sequence of SEQ ID NO: 33; and a Light Chain Variable Region (VL) comprising a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
d) a Heavy Chain Variable Region (VH) comprising a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 33; and a Light Chain Variable Region (VL) comprising a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
e) a Heavy Chain Variable Region (VH) comprising a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; and a Light Chain Variable Region (VL) comprising a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
f) a Heavy Chain Variable Region (VH) comprising a VH-CDR1 comprising the amino acid sequence of SEQ ID NO: 61, a VH-CDR2 comprising the amino acid sequence of SEQ ID NO: 62 and a VH-CDR3 comprising the amino acid sequence of SEQ ID NO: 63; and a Light Chain Variable Region (VL) comprising a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
g) a Heavy Chain Variable Region (VH) comprising a VH-CDR1 comprising the amino acid sequence of SEQ ID NO: 51, a VH-CDR2 comprising the amino acid sequence of SEQ ID NO: 52, and a VH-CDR3 comprising the amino acid sequence of SEQ ID NO: 53; and a Light Chain Variable Region (VL) comprising a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
h) a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
i) a Heavy Chain Variable Region (VH) comprising a VH-CDR1 comprising the amino acid sequence of SEQ ID NO: 111, a VH-CDR2 comprising the amino acid sequence of SEQ ID NO: 112 and a VH-CDR3 comprising the amino acid sequence of SEQ ID NO: 113; and a Light Chain Variable Region (VL) comprising a VL-CDR1 comprising the amino acid sequence of SEQ ID NO: 115, a VL-CDR2 comprising the amino acid sequence of SEQ ID NO: 116 and a VL-CDR3 comprising the amino acid sequence of SEQ ID NO: 117.

In another embodiment, a TDP-43 antibody comprises a Heavy Chain Variable Domain (VH) selected from SEQ ID NO: 10, 20, 30, 40, 50, 60, and 80 including post-translational modifications of that sequence. In a particular embodiment, the Heavy Chain Variable Domain (VH) comprises at least one, two, or three CDRs selected from (a) VH-CDR1 comprising the amino acid sequence selected from SEQ ID NO: 11, 21, 31, 41, 51, 61, and 81 (b) VH-CDR2 comprising the amino acid sequence selected from SEQ ID NO: 12, 22, 32, 42, 52, 62 and 82 (c) VH-CDR3 comprising the amino acid sequence selected from SEQ ID NO: 13, 23, 33, 53, 63 and 83.

In another embodiment, a TDP-43 antibody comprises a Heavy Chain Variable Domain (VH) selected from SEQ ID NO: 10, 20, 30, 40, 50, 60, 80 and 110 including post-translational modifications of that sequence. In a particular embodiment, the Heavy Chain Variable Domain (VH) comprises at least one, two, or three CDRs selected from (a) VH-CDR1 comprising the amino acid sequence selected from SEQ ID NO: 11, 21, 31, 41, 51, 61, 81 and 111 (b) VH-CDR2 comprising the amino acid sequence selected from SEQ ID NO: 12, 22, 32, 42, 52, 62, 82 and 112 (c) VH-CDR3 comprising the amino acid sequence selected from SEQ ID NO: 13, 23, 33, 53, 63, 83 and 113.

In another embodiment, a TDP-43 antibody comprises a Light Chain Variable Domain (VL) selected from SEQ ID NO: 14, 24, 34, 44, 54, and 84 including post-translational modifications of that sequence. In a particular embodiment, the Light Chain Variable Domain (VL) comprises at least one, two, or three CDRs selected from (a) VL-CDR1 comprising the amino acid sequence selected from SEQ ID NO: 15, 25, 35, 45, 55, 75 and 85 (b) VL-CDR2 comprising the amino acid sequence selected from SEQ ID NO: 16, 26, 46 and 76 (c) VL-CDR3 comprising the amino acid sequence selected from SEQ ID NO: 17, 27, 37, 47, 57, 77 and 87.

In another embodiment, a TDP-43 antibody comprises a Light Chain Variable Domain (VL) selected from SEQ ID NO: 14, 24, 34, 44, 54, 64, 74, 84 and 114 including post-translational modifications of that sequence. In a particular embodiment, the Light Chain Variable Domain (VL) comprises at least one, two, or three CDRs selected from (a) VL-CDR1 comprising the amino acid sequence selected from SEQ ID NO: 15, 25, 35, 45, 55, 75, 85 and 115 (b) VL-CDR2 comprising the amino acid sequence selected from SEQ ID NO: 16, 26, 46, 76 and 116 (c) VL-CDR3 comprising the amino acid sequence selected from SEQ ID NO: 17, 27, 37, 47, 57, 77, 87 and 117.

In some embodiments, the TDP-43 antibody comprises:
a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10 or
b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; or
c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; or
d. a Heavy Chain Variable Region (VH) comprising the sequence of SEQ ID NO: 40 or a Heavy Chain Variable Region (VH) having at least 98% or 99% sequence identity to the amino acid sequence of SEQ ID NO: 40; or
e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; or
f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; or
g. a Heavy Chain Variable Region (VH) comprising the sequence of SEQ ID NO: 80 or a Heavy Chain Variable Region (VH) having at least 94%, 95%, 96%, 97%, 98% or 99% sequence identity to the amino acid sequence of SEQ ID NO: 80.

In some embodiments, the TDP-43 antibody comprises:
a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10 or
b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; or
c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; or
d. a Heavy Chain Variable Region (VH) comprising the sequence of SEQ ID NO: 40 or a Heavy Chain Variable Region (VH) having at least 98% or 99% sequence identity to the amino acid sequence of SEQ ID NO: 40; or
e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; or
f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; or
g. a Heavy Chain Variable Region (VH) comprising the sequence of SEQ ID NO: 80 or a Heavy Chain Variable Region (VH) having at least 94%, 95%, 96%, 97%, 98% or 99% sequence identity to the amino acid sequence of SEQ ID NO: 80; or
h. a Heavy Chain Variable Region (VH) comprising the sequence of SEQ ID NO: 110 or a Heavy Chain Variable Region (VH) having at least 92%, 93%, 94%, 95%, 96%, 97%, 98% or 99% sequence identity to the amino acid sequence of SEQ ID NO: 110.

In some embodiments, the TDP-43 antibody comprises:
a. a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
b. a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
c. a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
e. a Light Chain Variable Region (VL) comprising the sequence of SEQ ID NO: 54; or
f. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84.

In some embodiment, the TDP-43 binding molecule, in particular a TDP-43 antibody or antigen-binding fragment comprises:
a) a Heavy Chain Variable Region (VH) selected from:
   i. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10 or
   ii. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; or
   iii. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; or
   iv. a Heavy Chain Variable Region (VH) comprising the sequence of SEQ ID NO: 40 or a Heavy Chain Variable Region (VH) having at least 98% or 99% sequence identity to the amino acid sequence of SEQ ID NO: 40; or
   v. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; or
   vi. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; or
   vii. a Heavy Chain Variable Region (VH) comprising the sequence of SEQ ID NO: 80 or a Heavy Chain Variable Region (VH) having at least 94%, 95%, 96%, 97%, 98% or 99% sequence identity to the amino acid sequence of SEQ ID NO: 80; and
b) a Light Chain Variable Region (VL) selected from:
   i. a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
   ii. a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
   iii. a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
   iv. a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
   v. a Light Chain Variable Region (VL) comprising the sequence of SEQ ID NO: 54; or
   vi. a Light Chain Variable Region (VL) comprising the sequence of SEQ ID NO: 64; or
   vii. a Light Chain Variable Region (VL) comprising the sequence of SEQ ID NO: 74; or
   viii. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84.

In some embodiment, the TDP-43 binding molecule, in particular a TDP-43 antibody or antigen-binding fragment comprises:
a) a Heavy Chain Variable Region (VH) selected from:
   i. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10 or
   ii. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; or
   iii. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; or
   iv. a Heavy Chain Variable Region (VH) comprising the sequence of SEQ ID NO: 40 or a Heavy Chain Variable Region (VH) having at least 98% or 99% sequence identity to the amino acid sequence of SEQ ID NO: 40; or
   v. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; or
   vi. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; or
   vii. a Heavy Chain Variable Region (VH) comprising the sequence of SEQ ID NO: 80 or a Heavy Chain Variable Region (VH) having at least 94%, 95%, 96%, 97%, 98% or 99% sequence identity to the amino acid sequence of SEQ ID NO: 80; or
   viii. a Heavy Chain Variable Region (VH) comprising the sequence of SEQ ID NO: 110 or a Heavy Chain Variable Region (VH) having at least 92%, 93%, 94%, 95%, 96%, 97%, 98% or 99% sequence identity to the amino acid sequence of SEQ ID NO: 110; and
b) a Light Chain Variable Region (VL) selected from:
   i. a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
   ii. a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
   iii. a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
   iv. a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
   v. a Light Chain Variable Region (VL) comprising the sequence of SEQ ID NO: 54; or
   vi. a Light Chain Variable Region (VL) comprising the sequence of SEQ ID NO: 64; or
   vii. a Light Chain Variable Region (VL) comprising the sequence of SEQ ID NO: 74; or
   viii. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
   ix. a Light Chain Variable Region (VL) comprising the sequence of SEQ ID NO: 114.

In some embodiments, the TDP-43 binding molecule, in particular a TDP-43 antibody or antigen-binding fragment thereof comprises:
a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10; and a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Heavy Chain Variable Region (VH) comprising the sequence of SEQ ID NO: 40 or a Heavy Chain Variable Region (VH) having at 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 54; or
f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; and a Light Chain Variable Region (VL) comprising the sequence of SEQ ID NO: 64; or
g. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 74; or
h. a Heavy Chain Variable Region (VH) comprising the sequence of SEQ ID NO: 80 or a Heavy Chain Variable Region (VH) having at least 94%, 95%, 96%, 97%, 98%, or 99% sequence identity to the amino acid sequence of SEQ ID NO: 80; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84.

In some embodiments, the TDP-43 binding molecule, in particular a TDP-43 antibody or antigen-binding fragment thereof comprises:
a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10; and a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Heavy Chain Variable Region (VH) comprising the sequence of SEQ ID NO: 40 or a Heavy Chain Variable Region (VH) having at 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 54; or
f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; and a Light Chain Variable Region (VL) comprising the sequence of SEQ ID NO: 64; or
g. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 74; or
h. a Heavy Chain Variable Region (VH) comprising the sequence of SEQ ID NO: 80 or a Heavy Chain Variable Region (VH) having at least 94%, 95%, 96%, 97%, 98%, or 99% sequence identity to the amino acid sequence of SEQ ID NO: 80; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
   i. a Heavy Chain Variable Region (VH) comprising the sequence of SEQ ID NO: 110 or a Heavy Chain Variable Region (VH) having at least 92%, 93%, 94%, 95%, 96%, 97%, 98%, or 99% sequence identity to the amino acid sequence of SEQ ID NO: 110; and a Light Chain Variable Region (VL) comprising the sequence of SEQ ID NO: 114.

In some embodiments, the TDP-43 antibody comprises:
a. a Heavy Chain Variable Region (VH) comprising the sequence of SEQ ID NO: 10 and a Light Chain Variable Region (VL) comprising the sequence of SEQ ID NO: 14; or
b. a Heavy Chain Variable Region (VH) comprising the sequence of SEQ ID NO: 20 and a Light Chain Variable Region (VL) comprising the sequence of SEQ ID NO: 24; or
c. a Heavy Chain Variable Region (VH) comprising the sequence of SEQ ID NO: 30 and a Light Chain Variable Region (VL) comprising the sequence of SEQ ID NO: 34; or
d. a Heavy Chain Variable Region (VH) comprising the sequence of SEQ ID NO: 40 and a Light Chain Variable Region (VL) comprising the sequence of SEQ ID NO: 44; or
e. a Heavy Chain Variable Region (VH) comprising the sequence of SEQ ID NO: 50 and a Light Chain Variable Region (VL) comprising the sequence of SEQ ID NO: 54; or
f. a Heavy Chain Variable Region (VH) comprising the sequence of SEQ ID NO: 60 and a Light Chain Variable Region (VL) comprising the sequence of SEQ ID NO: 64; or
g. a Heavy Chain Variable Region (VH) comprising the sequence of SEQ ID NO: 50 and a Light Chain Variable Region (VL) comprising the sequence of SEQ ID NO: 74; or
h. a Heavy Chain Variable Region (VH) comprising the sequence of SEQ ID NO: 80 and a Light Chain Variable Region (VL) comprising the sequence of SEQ ID NO: 84.

In some embodiments, the TDP-43 antibody comprises:
a. a Heavy Chain Variable Region (VH) comprising the sequence of SEQ ID NO: 10 and a Light Chain Variable Region (VL) comprising the sequence of SEQ ID NO: 14; or
b. a Heavy Chain Variable Region (VH) comprising the sequence of SEQ ID NO: 20 and a Light Chain Variable Region (VL) comprising the sequence of SEQ ID NO: 24; or
c. a Heavy Chain Variable Region (VH) comprising the sequence of SEQ ID NO: 30 and a Light Chain Variable Region (VL) comprising the sequence of SEQ ID NO: 34; or
d. a Heavy Chain Variable Region (VH) comprising the sequence of SEQ ID NO: 40 and a Light Chain Variable Region (VL) comprising the sequence of SEQ ID NO: 44; or
e. a Heavy Chain Variable Region (VH) comprising the sequence of SEQ ID NO: 50 and a Light Chain Variable Region (VL) comprising the sequence of SEQ ID NO: 54; or
f. a Heavy Chain Variable Region (VH) comprising the sequence of SEQ ID NO: 60 and a Light Chain Variable Region (VL) comprising the sequence of SEQ ID NO: 64; or
g. a Heavy Chain Variable Region (VH) comprising the sequence of SEQ ID NO: 50 and a Light Chain Variable Region (VL) comprising the sequence of SEQ ID NO: 74; or
h. a Heavy Chain Variable Region (VH) comprising the sequence of SEQ ID NO: 80 and a Light Chain Variable Region (VL) comprising the sequence of SEQ ID NO: 84; or
   i. a Heavy Chain Variable Region (VH) comprising the sequence of SEQ ID NO: 110 and a Light Chain Variable Region (VL) comprising the sequence of SEQ ID NO: 114.

In a preferred embodiment, the TDP-43 antibody comprises:
a. a Heavy Chain Variable Region (VH) comprising the sequence of SEQ ID NO: 10 and a Light Chain Variable Region (VL) comprising the sequence of SEQ ID NO: 14; or
b. a Heavy Chain Variable Region (VH) comprising the sequence of SEQ ID NO: 20 and a Light Chain Variable Region (VL) comprising the sequence of SEQ ID NO: 24; or
c. a Heavy Chain Variable Region (VH) comprising the sequence of SEQ ID NO: 30 and a Light Chain Variable Region (VL) comprising the sequence of SEQ ID NO: 34; or
d. a Heavy Chain Variable Region (VH) comprising the sequence of SEQ ID NO: 60 and a Light Chain Variable Region (VL) comprising the sequence of SEQ ID NO: 64.

In some embodiments, the invention relates to the TDP-43 binding molecule selected from ACI-8071-943.12A8-Ab1, ACI-8071-943.7H9-Ab1, ACI-8071-943.7D3-Ab1, ACI-8071-943.2E6-Ab1, ACI-8072-946.8H6-Ab1, ACI-8072-946.4G5-Ab1, ACI-8072-946.9D6-Ab1, ACI-7071-4665-B5-R3B-Ab2. Preferably, the TDP-43 binding molecule is selected from ACI-8071-943.12A8-Ab1, ACI-8071-943.7H9-Ab1, ACI-8071-943.7D3-Ab1, ACI-8072-946.4G5-Ab1.

Specifically, ACI-8071-943.12A8-Ab1 and ACI-8071-943.7D3-Ab1 may be useful as therapeutic antibodies. ACI-8071-943.7H9-Ab1 and ACI-8072-946.4G5-Ab1 may be useful as diagnostic/detection antibodies, for example in pairing assays.

In some embodiments, the invention relates to the TDP-43 binding molecule selected from ACI-8071-943.12A8-Ab1, ACI-8071-943.7H9-Ab1, ACI-8071-943.7D3-Ab1, ACI-8071-943.2E6-Ab1, ACI-8072-946.8H6-Ab1, ACI-8072-946.4G5-Ab1, ACI-8072-946.9D6-Ab1, ACI-7071-4665-B5-R3B-Ab2, ACI-8070-942.30D12-Ab1. In some embodiments preferably, ACI-7071-4665-B5-R3B-Ab2, ACI-7071-809F12-Ab1-rec2, ACI-8072-946.4G5-Ab1 and ACI-8071-943.7H9-Ab1 may be useful as diagnostic/detection antibodies, for example in pairing assays.

In some embodiments, a(n isolated) nucleic acid is provided, wherein the (isolated) nucleic acid encodes a TDP-43 binding molecule in particular TDP-43 antibody and fragment thereof described herein.

In some embodiments, a(n isolated) nucleic acid is provided, wherein the (isolated) nucleic acid comprises SEQ ID NO: 18 encoding a Heavy Chain Variable Region (VH) of an anti-TPD-43 antibody.

In some embodiments, a(n isolated) nucleic acid is provided, wherein the (isolated) nucleic acid comprises SEQ ID NO: 19 encoding a Light Chain Variable Region (VL) of an anti-TPD-43 antibody.

In some embodiments, a(n isolated) nucleic acid is provided, wherein the (isolated) nucleic acid comprises SEQ ID NO: 28 encoding a Heavy Chain Variable Region (VH) of an anti-TPD-43 antibody.

In some embodiments, a(n isolated) nucleic acid is provided, wherein the (isolated) nucleic acid comprises SEQ ID NO: 29 encoding a Light Chain Variable Region (VL) of an anti-TPD-43 antibody.

In some embodiments, a(n isolated) nucleic acid is provided, wherein the (isolated) nucleic acid comprises SEQ ID NO: 38 encoding a Heavy Chain Variable Region (VH) of an anti-TPD-43 antibody.

In some embodiments, a(n isolated) nucleic acid is provided, wherein the (isolated) nucleic acid comprises SEQ ID NO: 39 encoding a Light Chain Variable Region (VL) of an anti-TPD-43 antibody.

In some embodiments, a(n isolated) nucleic acid is provided, wherein the (isolated) nucleic acid comprises SEQ ID NO: 48 encoding a Heavy Chain Variable Region (VH) of an anti-TPD-43 antibody.

In some embodiments, a(n isolated) nucleic acid is provided, wherein the (isolated) nucleic acid comprises SEQ ID NO: 49 encoding a Light Chain Variable Region (VL) of an anti-TPD-43 antibody.

In some embodiments, a(n isolated) nucleic acid is provided, wherein the (isolated) nucleic acid comprises SEQ ID NO: 58 encoding a Heavy Chain Variable Region (VH) of an anti-TPD-43 antibody.

In some embodiments, a(n isolated) nucleic acid is provided, wherein the (isolated) nucleic acid comprises SEQ ID NO: 59 encoding a Light Chain Variable Region (VL) of an anti-TPD-43 antibody.

In some embodiments, a(n isolated) nucleic acid is provided, wherein the (isolated) nucleic acid comprises SEQ ID NO: 68 encoding a Heavy Chain Variable Region (VH) of an anti-TPD-43 antibody.

In some embodiments, a(n isolated) nucleic acid is provided, wherein the (isolated) nucleic acid comprises SEQ ID NO: 69 encoding a Light Chain Variable Region (VL) of an anti-TPD-43 antibody.

In some embodiments, a(n isolated) nucleic acid is provided, wherein the (isolated) nucleic acid comprises SEQ ID NO: 79 encoding a Light Chain Variable Region (VL) of an anti-TPD-43 antibody.

In some embodiments, a(n isolated) nucleic acid is provided, wherein the (isolated) nucleic acid comprises SEQ ID NO: 88 encoding a Heavy Chain Variable Region (VH) of an anti-TPD-43 antibody.

In some embodiments, a(n isolated) nucleic acid is provided, wherein the (isolated) nucleic acid comprises SEQ ID NO: 89 encoding a Light Chain Variable Region (VL) of an anti-TPD-43 antibody.

In some embodiments, a(n isolated) nucleic acid is provided, wherein the (isolated) nucleic acid comprises SEQ ID NO: 118 encoding a Heavy Chain Variable Region (VH) of an anti-TPD-43 antibody.

In some embodiments, a(n isolated) nucleic acid is provided, wherein the (isolated) nucleic acid comprises SEQ ID NO: 119 encoding a Light Chain Variable Region (VL) of an anti-TPD-43 antibody.

### Compositions and Methods

The invention also relates to pharmaceutical compositions comprising a TDP-43 binding molecule, particularly an antibody or an antigen-binding fragment thereof, of the invention as described herein and a pharmaceutically acceptable carrier and/or excipient and/or diluent.

In some embodiments, a pharmaceutical composition is provided, comprising an (isolated) antibody described herein and a pharmaceutically acceptable carrier.

In some embodiments, a conjugated binding molecule, in particular antibody or antigen-binding fragment thereof, is provided, comprising a binding molecule, in particular an antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), paramagnetic beads, nucleic acid molecules, detectable labels, therapeutic agents, toxins and blood brain barrier penetration moieties. In one embodiment of the invention, immunoconjugate comprises paramagnetic beads (capture antibody). In another embodiment of the invention, the immunoconjugate comprises biotin (detect antibody). Conjugation methods are well known in the art and several technologies are commercially available for conjugating antibodies to a label or other molecule, Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method.

Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Conjugation may be via a suitable linker moiety to ensure suitable spatial separation of the antibody and conjugated molecule, such as detectable label. However, a linker may not be required in all instances. In some embodiments the TDP-43 binding molecule of the present invention is linked to a detectable label.

The invention also relates to an immunoconjugate comprising the TDP-43 binding molecule provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood brain barrier penetration moieties or detectable labels. Various techniques exist for improving drug delivery across the blood-brain barrier (BBB) as discussed herein, which discussion applies mutatis mutandis. Non-invasive techniques include the so-called “Trojan horse approach” in which conjugated molecules deliver the binding molecules of the invention by binding to BBB receptors and mediating transport. Suitable molecules may comprise endogenous ligands or antibodies, in particular monoclonal antibodies, that bind specific epitopes on the BBB receptor.

The invention further comprises the use of the antibody of the invention in a pairing assay, such as an immunoassay or an ELISA to detect or quantify phosphorylated TDP-43 in a human sample. Reference may be made to Examples 8 for a detailed description of preferred pairing assays that may be employed. Such an assay comprises the incubation of a sample with at least two different TDP-43 binding molecules of the invention. The two different TDP-43 binding molecules of the invention do not compete with each other for binding to TDP-43; they bind different epitopes. One binding molecule is typically a capture antibody, used to immobilize TDP-43, and the other binding molecule is a detect antibody, which binds to the immobilized TDP-43 and provides the signal for the assay, either directly (where the antibody is labelled) or indirectly (through a downstream signal generation system that may amplify the signal, for example enzymatically and may involve secondary antibodies binding to the detect antibody). The assay may be a digital assay, capable of providing a quantitative readout, in preferred embodiments.

In one embodiment, the assay comprises the steps of:
a. incubating the sample with a capture antibody, which may be a TDP-43 binding molecule of the invention conjugated to paramagnetic beads;
b. adding to the solution of a) a detect antibody, which may be a labeled TDP-43 binding molecule of the invention, such as an antibody of the invention conjugated to biotin;
c. optionally adding a substrate for the labeled TDP-43 binding molecule, such as Streptavidin-β-D-Galactosidase;
d. optionally washing the plate using a magnetic microplate washer;
e. reading the signal emitted by the labeled TDP-43 binding molecule.

In one embodiment, the capture antibody is an immunoconjugate or a TDP-43 binding molecule, in particular an antibody or antigen-binding fragment thereof, described herein conjugated to paramagnetic beads. In a preferred embodiment, the capture antibody binds specifically to phosphorylated TDP-43. In another preferred embodiment, the capture antibody of the invention binds misfolded aggregated TDP-43 and non-aggregated physiological TDP-43.

In a preferred embodiment, the capture antibody is a TDP-43 binding molecule selected from the group of TDP-43 binding molecules comprising:
a. a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
b. a Heavy Chain Variable Region (VH) comprising a VH-CDR1 comprising the amino acid sequence of SEQ ID NO: 91, a VH-CDR2 comprising the amino acid sequence of SEQ ID NO: 92 and a VH-CDR3 comprising the amino acid sequence of SEQ ID NO: 93; and a Light Chain Variable Region (VL) comprising a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
c. a Heavy Chain Variable Region (VH) comprising a VH-CDR1 comprising the amino acid sequence of SEQ ID NO: 101, a VH-CDR2 comprising the amino acid sequence of SEQ ID NO: 102 and a VH-CDR3 comprising the amino acid sequence of SEQ ID NO: 103; and a Light Chain Variable Region (VL) comprising a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.

In a more preferred embodiment, the capture antibody comprises a TDP-43 binding molecule comprising a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.

In a preferred embodiment, the detect antibody binds specifically to phosphorylated TDP-43. In another preferred embodiment, the detect antibody of the invention binds misfolded aggregated TDP-43 and non-aggregated physiological TDP-43.

In a preferred embodiment, the capture antibody binds specifically to phosphorylated TDP-43. In another preferred embodiment, the capture antibody of the invention binds misfolded aggregated TDP-43 and non-aggregated physiological TDP-43.

In a preferred embodiment, the detect antibody is a TDP-43 binding molecule selected from the group of TDP-43 binding molecules comprising:
a. a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
b. a Heavy Chain Variable Region (VH) comprising a VH-CDR1 comprising the amino acid sequence of SEQ ID NO: 91, a VH-CDR2 comprising the amino acid sequence of SEQ ID NO: 92 and a VH-CDR3 comprising the amino acid sequence of SEQ ID NO: 93; and a Light Chain Variable Region (VL) comprising a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
c. a Heavy Chain Variable Region (VH) comprising a VH-CDR1 comprising the amino acid sequence of SEQ ID NO: 101, a VH-CDR2 comprising the amino acid sequence of SEQ ID NO: 102 and a VH-CDR3 comprising the amino acid sequence of SEQ ID NO: 103; and a Light Chain Variable Region (VL) comprising a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107; or
d. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
e. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57.

In a preferred embodiment, the detect antibody is a TDP-43 binding molecule selected from the group of TDP-43 binding molecules comprising:
a. a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
b. a Heavy Chain Variable Region (VH) comprising a VH-CDR1 comprising the amino acid sequence of SEQ ID NO: 91, a VH-CDR2 comprising the amino acid sequence of SEQ ID NO: 92 and a VH-CDR3 comprising the amino acid sequence of SEQ ID NO: 93; and a Light Chain Variable Region (VL) comprising a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
c. a Heavy Chain Variable Region (VH) comprising a VH-CDR1 comprising the amino acid sequence of SEQ ID NO: 101, a VH-CDR2 comprising the amino acid sequence of SEQ ID NO: 102 and a VH-CDR3 comprising the amino acid sequence of SEQ ID NO: 103; and a Light Chain Variable Region (VL) comprising a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.

In a more preferred embodiment, the detect antibody comprises a TDP-43 binding molecule comprising a Heavy Chain Variable Region (VH) comprising a VH-CDR1 comprising the amino acid sequence of SEQ ID NO: 81, a VH-CDR2 comprising the amino acid sequence of SEQ ID NO: 82 and a VH-CDR3 comprising the amino acid sequence of SEQ ID NO: 83; and a Light Chain Variable Region (VL) comprising a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.

In one embodiment of the invention, the capture antibody binds specifically to phosphorylated TDP-43 and the detect antibody binds misfolded aggregated TDP-43 and non-aggregated physiological TDP-43. In another embodiment of the invention, the detect antibody binds misfolded aggregated TDP-43 and non-aggregated physiological TDP-43, and the capture antibody binds specifically to phosphorylated TDP-43. In one embodiment the capture antibody and the detect antibody have different epitopes.

In one embodiment, the detect antibody comprises a TDP-43 binding molecule comprising a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; and the capture antibody comprises a TDP-43 binding molecule comprising a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107. In a further embodiment, the pairing assay to detect or quantify TDP-43 with the defined capture and detect antibodies may provide a lower limit of quantification of 0.02 pM and/or a lower limit of detection of 0.09 pM.

In one embodiment, the detect antibody comprises a TDP-43 binding molecule comprising a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; and the capture antibody comprises a TDP-43 binding molecule comprising a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97. In a further embodiment, the pairing assay to detect or quantify phosphorylated TDP-43 with the defined capture and detect antibodies may provide a lower limit of quantification of 0.13 pM and/or a lower limit of detection of 0.5 pM.

In one embodiment, the detect antibody comprises a TDP-43 binding molecule comprising a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; and the capture antibody comprises a TDP-43 binding molecule comprising a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107. In a further embodiment, the pairing assay to detect or quantify phosphorylated TDP-43 with the defined capture and detect antibodies may provide a lower limit of quantification of 0.17 pM and/or a lower limit of detection of 0.61 pM.

In one embodiment, the detect antibody comprises a TDP-43 binding molecule comprising a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; and the capture antibody comprises a TDP-43 binding molecule comprising a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107. In a further embodiment, the pairing assay to detect or quantify phosphorylated TDP-43 with the defined capture and detect antibodies may provide a lower limit of quantification of 0.02 pM and/or a lower limit of detection of 0.8 pM.

In some embodiments, an immunoconjugate is provided, wherein the immunoconjugate comprises an (isolated) antibody described herein and a therapeutic agent. In some embodiments, a labeled antibody is provided, comprising an antibody described herein and a detectable label.

In some embodiments, the TDP-43 binding molecule is part of an immunoconjugate wherein the TDP-43 binding molecule is covalently linked to another suitable therapeutic agent.

In some embodiments, the TDP-43 binding molecule or the immunoconjugate comprising it is present as a composition comprising a TDP-43 binding molecule.

In some embodiments, the TDP-43 binding molecule is part of a pharmaceutical composition comprising a TDP-43 binding molecule or an immunoconjugate wherein the TDP-43 binding molecule is covalently linked to another suitable therapeutic agent, or a composition comprising a TDP-43 binding molecule combined with a pharmaceutically acceptable carrier and/or excipient and/or diluent.

In some embodiments, the TDP-43 binding molecule is part of a diagnostic composition comprising a TDP-43 binding molecule and an acceptable carrier and/or excipient and/or diluent.

In some embodiments, the TDP-43 binding molecule is part of a detection and/or diagnostic kit comprising a TDP-43 binding molecule, or an immunoconjugate wherein the TDP-43 binding molecule is covalently linked to another suitable therapeutic agent, or a composition comprising a TDP-43 binding molecule.

Kits containing the binding molecules of the invention are also provided. In particular, such kits may be used for diagnostic applications. Thus, a kit for diagnosis of a disease, disorder and/or abnormality associated with TDP-43, in particular associated with TDP-43 aggregates, or a TDP-43 proteinopathy, or for use in a method of the invention is provided comprising a TDP-43 binding molecule of the invention. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed. Suitably labelled TDP-43 binding molecules of the invention are also provided, which may be included in such kits.

In some embodiments the TDP-43 binding molecule is used in an immunodiagnostic method for the diagnosis of a TDP-43 proteinopathy. In some embodiment, the TDP-43 binding molecule is used as a diagnostic tool in combination with a therapeutic TDP-43 molecule.

In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used to diagnose, prevent, alleviate or treat a disease, disorder and/or abnormality associated with TDP-43, in particular associated with TDP-43 aggregates, or TDP-43 proteinopathy including but not limited to Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Alzheimer's Disease (AD), Down Syndrome (DS), Parkinson's Disease (PD) and related disorders (including PD with Dementia (PDD), dementia with Lewy Bodies (DLB), multiple system atrophy (MSA)), limbic-predominant age-related TDP-43 encephalopathy (LATE), myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy, and diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B genes.

In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used in a method for diagnosing or monitoring a disease, disorder and/or abnormality associated with TDP-43, in particular associated with TDP-43 aggregates, or TDP-43 proteinopathy selected from the group consisting of Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), diseases of both sporadic and hereditary origins, including genetic cases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

In other embodiment, the invention relates to any methods for detecting, diagnosing or monitoring a disease, disorder and/or abnormality associated with TDP-43, in particular associated with TDP-43 aggregates, or TDP-43 proteinopathy that is Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), diseases of both sporadic and hereditary origins, including genetic cases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

Preferably, the disease, disorder and/or abnormality associated with TDP-43, in particular associated with TDP-43 aggregates, or TDP-43 proteinopathy is selected from Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Alzheimer's Disease (AD), Down Syndrome (DS), Parkinson's Disease (PD) and related disorders (including PD with Dementia (PDD), dementia with Lewy Bodies (DLB), multiple system atrophy (MSA)), limbic-predominant age-related TDP-43 encephalopathy (LATE), myofibrillar myopathies (e.g. inclusion body myositis, inclusion body myopathy, oculopharyngeal muscular dystrophy with rimmed vacuoles), and diseases of both sporadic and hereditary origins, including genetic cases arising from mutations or variant-associated risk alleles of the progranulin (GRN), TARDBP, C9ORF72, valosin-containing protein (VCP), angiogenin (ANG), desmin (DES), myotilin (MYOT), TMEM106B genes. More preferably, the disease, disorder and/or abnormality associated with TDP-43, in particular associated with TDP-43 aggregates, or TDP-43 proteinopathy is amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND (also known as ALS-FTD).

The invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the TDP-43 levels in the samples, wherein;
a. a change of levels of TDP-43 in the later sample compared with one or more earlier samples is indicative of modification of a disease, disorder and/or condition associated with TDP-43; or
b. no significant change of levels of TDP-43 in the later sample compared with one or more earlier samples are indicative of lack of modification of a disease, disorder and/or condition associated with TDP-43.

The invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the phosphorylated TDP-43 levels in the samples, wherein;
a. a change of levels of phosphorylated TDP-43 in the later sample compared with one or more earlier samples is indicative of modification of a disease, disorder and/or condition associated with TDP-43; or
b. no significant change of levels of phosphorylated TDP-43 in the later sample compared with one or more earlier samples are indicative of lack of modification of a disease, disorder and/or condition associated with TDP-43.

The invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the TDP-43 levels in the samples, wherein:
a. higher levels of TDP-43 in the later sample compared with one or more earlier samples are indicative of progression of a disease, disorder and/or condition associated with TDP-43;
b. lower levels of TDP-43 in the later sample compared with one or more earlier samples are indicative of regression of a disease, disorder and/or condition associated with TDP-43; or
c. no significant change of levels of TDP-43 in the later sample compared with one or more earlier samples are indicative of a lack of progression of a disease, disorder and/or condition associated with TDP-43.

Alternatively, the invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the TDP-43 levels in the samples, wherein:
a. higher levels of TDP-43 in the later sample compared with one or more earlier samples are indicative of regression of a disease, disorder and/or condition associated with TDP-43;
b. lower levels of TDP-43 in the later sample compared with one or more earlier samples are indicative of progression of a disease, disorder and/or condition associated with TDP-43; or
c. no significant change of levels of TDP-43 in the later sample compared with one or more earlier samples are indicative of a lack of progression of a disease, disorder and/or condition associated with TDP-43.

The invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the phosphorylated TDP-43 levels in the samples, wherein:
a. higher levels of phosphorylated TDP-43 in the later sample compared with one or more earlier samples are indicative of progression of a disease, disorder and/or condition associated with TDP-43;
b. lower levels of phosphorylated TDP-43 in the later sample compared with one or more earlier samples are indicative of regression of a disease, disorder and/or condition associated with TDP-43; or
c. no significant change of levels of phosphorylated TDP-43 in the later sample compared with one or more earlier samples are indicative of a lack of progression of a disease, disorder and/or condition associated with TDP-43.

Alternatively, the invention also provides a method for monitoring a disease, disorder and/or condition associated with TDP-43 at two or more time points using samples from a subject, the method comprising contacting the samples with a binding molecule, in particular an antibody or antigen-binding fragment of the invention and comparing the phosphorylated TDP-43 levels in the samples, wherein:
a. higher levels of phosphorylated TDP-43 in the later sample compared with one or more earlier samples are indicative of regression of a disease, disorder and/or condition associated with TDP-43;
b. lower levels of phosphorylated TDP-43 in the later sample compared with one or more earlier samples are indicative of progression of a disease, disorder and/or condition associated with TDP-43; or
c. no significant change of levels of phosphorylated TDP-43 in the later sample compared with one or more earlier samples are indicative of a lack of progression of a disease, disorder and/or condition associated with TDP-43.

Such methods are typically performed in relation to subjects known to have the disease, disorder and/or condition associated with TDP-43, may be performed at multiple time points in matched samples between a treatment and placebo groups in order to monitor the effectiveness of a candidate therapy over a defined time period. Diagnostic compositions of the invention may be used in such methods. The pairing immunoassays described herein, incorporating a suitable capture and detection antibody or antigen binding fragment thereof, may be used in the monitoring methods of the invention.

In some embodiments the TDP-43 binding molecule is used in a method of quantifying phosphorylated TDP-43 in a sample, the method comprising contacting the sample with a TDP-43 binding molecule of the invention, comparing detected TDP-43 levels from the sample to a control, optionally determined using a known amount of calibrator for phosphorylated TDP-43. In one embodiment of the invention, the method of quantifying is based on a pairing assay as described herein.

In one embodiment, the method of quantifying comprises the steps of:
a. Incubating a sample with a capture antibody and a detect antibody as described herein;
b. Incubating the mixture obtained in step a with a reagent suitable for detection by the detect antibody;
c. Measuring the signal emitted by the detect antibody;
d. Comparing detected TDP-43 levels from the sample to a control, optionally determined using a known amount of calibrator for phosphorylated TDP-43

A calibrator may be a peptide, protein or a compound used as a standard or reference material of known concentration to allow quantification of analyte levels in patient. In one embodiment of the invention, the calibrator is a peptide which comprises, consists or essentially consists of, the epitope of the capture antibody and the epitope of the detect antibody. In a preferred embodiment, the calibrator is selected from the group consisting of full-length TDP-43 (SEQ ID NO: 1), Peptide 28 (SEQ ID NO: 7 linked to SEQ ID NO: 8 via a Trioxatridecan-succinamic acid linker) and Peptide 29 (SEQ ID NO: 7 linked to SEQ ID NO: 9 via a Trioxatridecan-succinamic acid linker). In one embodiment of the invention, the calibrator is a peptide which comprises, consists or essentially consists of, the epitope of the capture antibody and the epitope of the detect antibody. In a preferred embodiment, the calibrator is selected from the group consisting of full-length TDP-43 (SEQ ID NO: 1), Peptide 11 (SEQ ID NO: 4 linked to SEQ ID NO: 5 via a Trioxatridecan-succinamic acid linker) and Peptide 23 (SEQ ID NO: 6 linked to SEQ ID NO: 5 via a Trioxatridecan-succinamic acid linker).

In some embodiments the TDP-43 binding molecule is used in a method wherein a TDP-43 binding molecule of the present invention is contacted with a sample (e.g., blood, cerebrospinal fluid, interstitial fluid (ISF), or brain tissue) to detect, Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy.

In some embodiments the TDP-43 binding molecule is used in a method wherein a TDP-43 binding molecule of the present invention is contacted with a sample (e.g., blood, cerebrospinal fluid, interstitial fluid (ISF), or brain tissue) to detect diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes. In some embodiments the TDP-43 binding molecule is used in a method wherein a TDP-43 binding molecule of the present invention is contacted with a sample (e.g., blood, cerebrospinal fluid, interstitial fluid (ISF), or brain tissue) to detect amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND.

In some embodiments the TDP-43 binding molecule is used in a method wherein a TDP-43 binding molecule of the present invention is contacted with a sample (e.g., blood, cerebrospinal fluid, interstitial fluid (ISF), or brain tissue) to detect a disease, disorder, or abnormality associated with TDP-43 aggregates is selected from the group consisting of Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy. In some embodiments the TDP-43 binding molecule is used in a method wherein a TDP-43 binding molecule of the present invention is contacted with a sample (e.g., blood, cerebrospinal fluid, interstitial fluid (ISF), or brain tissue) to detect diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used for preventing, alleviating or treating a disease, disorder and/or abnormality associated with TDP-43, in particular associated with TDP-43 aggregates, or TDP-43 proteinopathies, or Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy. In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used for preventing, alleviating or treating diseases arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used for treating a disease selected from: Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy. In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used for treating a disease arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes. Preferably said disease treatment helps to retain or increase mental recognition and or reduces the level of TDP-43 aggregates in the brain.

In some embodiments the TDP-43 binding molecule, or an immunoconjugate wherein the TDP-43 binding molecule is covalently linked to another suitable therapeutic agent, or a composition comprising a TDP-43 binding molecule is administered to a subject in need thereof or is used for manufacturing a medicament for treating a disease, disorder and/or abnormality associated with TDP-43 defined according to the invention, in particular associated with TDP-43 aggregates, or TDP-43 proteinopathies or amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND.

Pharmaceutical formulations of an anti-TDP-43 antibody (the preferred type of TDP-43 binding molecule) or immunoconjugate as described herein are prepared by mixing such antibody or immunoconjugate having the desired degree of purity with one or more optional pharmaceutically acceptable carriers and/or excipients and/or diluents (Remington's Pharmaceutical Sciences 16^{th} edition, Osol, A. Ed. (1980)). Typically, the antibody or fragment thereof is prepared as a lyophilized formulation or aqueous solution. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and lanolin. Diluents may be buffers. They may comprise a salt selected from the group consisting of phosphate, acetate, citrate, succinate and tartrate, and/or wherein the buffer comprises histidine, glycine, TRIS glycine, Tris, or mixtures thereof. It is further envisaged in the context of the present invention that the diluent is a buffer selected from the group consisting of potassium phosphate, acetic acid/sodium acetate, citric acid/sodium citrate, succinic acid/sodium succinate, tartaric acid/sodium tartrate, and histidine/histidine HCI or mixtures thereof.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules. The formulations to be used for in vivo administration are generally sterile. Sterility may be readily accomplished, e.g., by filtration through sterile filtration membranes.

Any of the antigen-binding molecules, anti-TDP-43 antibodies or immunoconjugates provided herein may be used in methods, e.g., diagnostic methods or therapeutic methods. The TDP-43 binding molecules of the invention may be used in methods of treatment, or for use as in the preparation of a medicament to be used in methods of treatment, or for use in the treatment of diseases as defined according to the invention.

In another aspect, an anti-TDP-43 antibody (the preferred type of TDP-43 binding molecule) or immunoconjugate for use as a medicament is provided. In further aspects, an anti-phosphorylated TDP-43 antibody (the preferred type of TDP-43 binding molecule) or immunoconjugate for use in a method of treatment is provided. In certain embodiments, an anti-TDP-43 antibody (the preferred type of TDP-43 binding molecule) or immunoconjugate for use in the prevention, diagnosis and/or treatment of a TDP-43 proteinopathy is provided. In a preferred embodiment of the invention, an anti-TDP-43 antibody (the preferred type of TDP-43 binding molecule) or immunoconjugate is provided for use in the prevention, diagnosis and/or treatment of a disease, disorder and/or abnormality associated with TDP-43, in particular associated with TDP-43 aggregates, or TDP-43 proteinopathy including but not limited to amyotrophic lateral sclerosis (ALS), Frontotemporal dementia (FTD), limbic-predominant age-related TDP-43 encephalopathy (LATE), Alzheimer's disease (AD), Frontotemporal Lobar Degeneration with Motor Neuron Disease FTLD-MND (also known as ALS-FTD).

In a further aspect, the invention provides for the use of an anti-TDP-43 antibody (the preferred type of TDP-43 binding molecule) or immunoconjugate in the manufacture or preparation of a medicament. In one such embodiment, the method further comprises administering to the individual an effective amount of at least one additional therapeutic agent, e.g., as described below.

A “subject” or an “individual” according to any of the embodiments may be an animal, a mammal, preferably a human.

In a further aspect, the invention provides pharmaceutical formulations comprising any of the anti-TDP-43 antibodies (the preferred type of TDP-43 binding molecule) or immunoconjugate provided herein, e.g., for use in any of the therapeutic methods. In one embodiment, a pharmaceutical formulation comprises any of the anti-TDP-43 antibodies (the preferred type of TDP-43 binding molecule) or immunoconjugates provided herein and a pharmaceutically acceptable carrier and/or excipients and/or diluents (as discussed elsewhere herein). In another embodiment, a pharmaceutical formulation comprises any of the anti-TDP-43 antibodies (the preferred type of TDP-43 binding molecule) or immunoconjugates provided herein and at least one additional therapeutic agent, e.g., as described below.

Antibodies or immunoconjugates of the invention can be used either alone or in combination with other agents in a therapy. For instance, an antibody (the preferred type of TDP-43 binding molecule) or immunoconjugate of the invention may be co-administered with at least one additional therapeutic agent targeting alpha-synuclein, BACE1, Tau, beta-amyloid, TDP-43 or a neuroinflammation protein.

Such combination therapies noted above encompass combined administration (where two or more therapeutic agents are included in the same or separate formulations), and separate administration, in which case, administration of the antibody (the preferred type of TDP-43 binding molecule) or immunoconjugate of the invention can occur prior to, simultaneously, and/or following, administration of the additional therapeutic agent and/or adjuvant. Antibodies (the preferred type of TDP-43 binding molecule of the invention) or immunoconjugates of the invention can also be used in combination with radiation therapy.

An antibody (the preferred type of TDP-43 binding molecule) or immunoconjugate of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies (the preferred type of TDP-43 binding molecule) or immunoconjugates of the invention would be formulated, dosed, and administered in a fashion consistent with good medical practice. Factors for consideration in this context include the particular disease, disorder and/or abnormality associated with TDP-43, in particular associated with TDP-43 aggregates, or TDP-43 proteinopathy being treated, the particular mammal being treated, the clinical condition of the individual subject, the cause of the disease, disorder and/or abnormality associated with TDP-43, in particular associated with TDP-43 aggregates, or TDP-43 proteinopathy, the site of delivery of the agent, the method of administration, the scheduling of administration, and other factors known to medical practitioners. The antibody or immunoconjugate need not be, but is optionally formulated with one or more agents currently used to prevent or treat the disease, disorder and/or abnormality associated with TDP-43, in particular associated with TDP-43 aggregates, or TDP-43 proteinopathy in question. The effective amount of such other agents depends on the amount of antibody or immunoconjugate present in the formulation, the type of disease, disorder and/or abnormality associated with TDP-43, in particular associated with TDP-43 aggregates or TDP-43 proteinopathy, or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody (the preferred type of TDP-43 binding molecule) or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the subject's clinical history and response to the antibody or immunoconjugate, and the discretion of the attending physician. The antibody (the preferred type of TDP-43 binding molecule) or immunoconjugate is suitably administered to the subject at one time or over a series of treatments. Depending on the type and severity of the disease, about 1 μg/kg to 15 mg/kg (e.g. 0.1 mg/kg-10 mg/kg) of antibody (the preferred type of TDP-43 binding molecule) or immunoconjugate can be an initial candidate dosage for administration to the subject, whether, for example, by one or more separate administrations, or by continuous infusion. One typical daily dosage might range from about 1 g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using both an immunoconjugate of the invention and an anti-TDP-43 antibody (the preferred type of TDP-43 binding molecule).

In another aspect of the invention, an article of manufacture containing materials useful for the treatment, prevention and/or diagnosis of the diseases, disorders or abnormalities associated with TDP-43, in particular associated with TDP-43 aggregates, or TDP-43 proteinopathy, described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder and/or abnormality associated with TDP-43, in particular associated with TDP-43 aggregates, or TDP-43 proteinopathy, and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice.

Moreover, the article of manufacture may comprise (a) a first container with a composition contained therein, wherein the composition comprises an antibody (the preferred type of TDP-43 binding molecule) or immunoconjugate of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further embodiment, the invention relates to a method of retaining or increasing cognitive memory capacity, movement and language function or preventing and/or slowing decline of cognitive memory capacity, movement and language function in a subject, comprising administering the binding molecule of the invention, the immunoconjugate of the invention, the composition of the invention or the pharmaceutical composition of the invention.

In a further embodiment, the invention relates to a method of reducing the level of TDP-43, comprising administering the binding molecule of the invention, the immunoconjugate of the invention, the composition of the invention or the pharmaceutical composition of the invention.

The methods of the invention may comprise administering at least one additional therapeutic agent, for instance at least one additional therapeutic agent targeting alpha-synuclein, BACE1, tau, beta-amyloid, TDP-43 or a neuroinflammation protein.

The invention furthermore relates to a method of detecting TDP-43, comprising contacting a sample with a binding molecule of the invention, preferably an antibody of the invention wherein the sample is a biological sample from a subject, such as a sample selected from a brain sample, a cerebrospinal fluid sample, urine sample or a blood sample. In an embodiment, the method of detecting TDP-43 comprises contacting plasma derived from a blood sample with a binding molecule of the invention.

In another embodiment, the method of detecting TDP-43 comprises contacting platelets derived from a blood sample with a binding molecule of the invention.

In one preferred embodiment, the method of detecting TDP-43 comprises contacting the cytosolic fraction of platelets derived from a blood sample with a binding molecule of the invention.

Reference can be made to Example 9 for a method of detecting TDP-43 in platelets derived from a blood sample.

In certain embodiments, the TDP-43 binding molecule, in particular TDP-43 antibody and fragment thereof as provided herein has a dissociation constant (KD) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M), in particular with respect to binding TDP-43, in particular soluble TDP-43. For example, the TDP-43 binding molecules of the invention may have a KD for phosphorylated TDP-43 of 13 nM or less, in specific embodiments 10 nM or 5 nM or less, in more specific embodiments a KD for phosphorylated TDP-43 of 2 nM or less. This is demonstrated for TDP-43 binding molecules of the invention in Example 5 with reference to Table 7 for binding to peptide 28 (SEQ ID NO: 7 linked to SEQ ID NO: 8 via a Trioxatridecan-succinamic acid linker).

In another example, the TDP-43 binding molecules of the invention may have a KD for phosphorylated TDP-43 of 3.5 nM or less, in specific embodiments 2.5 nM or less, in more specific embodiments a KD for phosphorylated TDP-43 of 1.7 nM or less. This is demonstrated for TDP-43 binding molecules of the invention in Example 5 with reference to Table 7 for binding to peptide 29 (SEQ ID NO: 7 linked to SEQ ID NO: 9 via a Trioxatridecan-succinamic acid linker). In yet another example, the TDP-43 binding molecules of the invention may have a KD for phosphorylated TDP-43 of 25 nM or less, in specific embodiments 21.5 nM or less. This is demonstrated for TDP-43 binding molecules of the invention in Example 5 with reference to Table 8 for binding to human TDP-43 amino acid positions 370-384 with pS375 and pS379 (SEQ ID NO: 5).

In one embodiment, binding affinity to full length (FL) TDP-43 or peptides of TDP-43 (such as peptide 28, SEQ ID NO: 7 linked to SEQ ID NO: 8 via a Trioxatridecan-succinamic acid linker, or peptide 29, SEQ ID NO: 7 linked to SEQ ID NO: 9 via a Trioxatridecan-succinamic acid linker) may be evaluated by determining the dissociation constants (KD) using surface plasmon resonance (SPR; Biacore 8K, GE Healthcare Life Sciences). Reference may be made to Example 5 for a detailed description of suitable SPR methods that may be employed.

In another embodiment, binding affinity to full length (FL) TDP-43 or peptides of TDP-43 (such as the peptide consisting of the amino acid sequence SEQ ID NO: 5) may be evaluated by determining the dissociation constants (KD) using surface plasmon resonance (SPR; Biacore 8K, GE Healthcare Life Sciences). Reference may be made to Example 5 for a detailed description of suitable SPR methods that may be employed.

In one embodiment, the TDP-43 binding molecule binds human TDP-43 (SEQ ID NO: 1) phosphorylated at positions pS375 and/or pS379. The TDP-43 binding molecule may bind TDP-43 (SEQ ID NO: 1) phosphorylated at positions pS375 and pS379 with a KD of 0.23 nM or less; and/or the TDP-43 binding molecule may bind TDP-43 (SEQ ID NO: 1) phosphorylated at position pS375 with a KD of 75.5 nM or less; and/or the TDP-43 binding molecule may bind TDP-43 (SEQ ID NO: 1) phosphorylated at position pS379 with a KD of 0.28 nM or less. Preferably the KD is measured by surface plasmon resonance. Reference can be made to Example 6 using phosphorylated peptides 370-384 as a suitable method to determine the KD by surface plasmon resonance.

### BRIEF DESCRIPTION OF FIGURES

FIG. 1. Immunoassay pairing antibodies ACI-7071-4665-B5-R3B-Ab2 and ACI-7071-809F12-Ab1-rec2 to measure human TDP-43 in plasma samples from healthy control and FTLD-TDP patients using SIMOA® assay technology. (A) Calibration curves using human TDP-43 were run on separate days establishing a Limit of Detection (LOD) of 0.021 pM and LLOQ of 0.09 pM (B). TDP-43 was measured in human plasma from healthy control and FTLD-TDP (semantic dementia, C9ORF72, or GRN) patients

FIG. 2. Graphical representation of the TDP-43 protein level measured in platelet-poor and platelet-rich plasma, and in the soluble and insoluble fractions of the platelet-rich plasma measured by an immunoassay pairing antibodies ACI-7071-4665-B5-R3B-Ab2 and ACI-7071-809F12-Ab1-rec2 using SIMOA® assay technology.

### EXAMPLES

### Example 1: Mouse Immunizations to Generate TDP-43 and Phospho TDP-43 Antibodies

Female wild-type C57BL/6JolaHsd (C57BL/6) and BALB/c OlaHsd (BALB/c) mice (Harlan, U.S.A.) were vaccinated at 10 weeks of age.

For antibodies generated using a liposome-based vaccine, the vaccine formulation was prepared according to methods described and published in WO2012/055933, modified to use the full-length human TDP-43 (FL TDP-43) protein as the antigen.

For generating TDP-43 antibodies, mice were vaccinated with full-length TDP-43 protein presented on the surface of liposomes in the presence of monophosphoryl hexa-acyl lipid-A, 3-deacyl (Synthetic) (3D-(6-acyl) PHAD®) as adjuvant. Mice were vaccinated by subcutaneous (s.c.) injections on days 0, 4, 8, 21, 35, and 60. Mice were bled and heparinized plasma prepared 7 days before immunization (pre-immune plasma) and on days 14, 28, 42, 81 and 121 after first immunization. Vaccine responses were measured by performing ELISAs on plasma antibodies using immobilized FL TDP-43 as the target. Mice to be used for generating an immune scFv phage library were additionally vaccinated with three daily intraperitoneal (i.p.) adjuvant-free booster injections prior to cell harvest.

For antibodies targeting phospho TDP-43, mice were vaccinated with antigen peptides representing human TDP-43 amino acids 396-409 carrying phosphorylated Serine (“phospho Ser”) at positions 403 and 404 (pS403 and pS404, respectively), and 401-413 carrying phospho Ser at 409 and 410 (pS409 and pS410, respectively). Peptide antigens were coupled to keyhole limpet haemocyanin (KLH) via maleimide conjugation using (m-maleimidobenzoyl-N-hydroxy)sulfo succinimide ester. A mixture of aluminium hydroxide gel (Alhydrogel adjuvant; Invivogen, France) and synthetic oligodeoxynucleotides (CpG ODN 1668; Microsynth, Switzerland) was used as an adjuvant. Vaccinations were done via s.c. injection into each limb and neck on days 0, 7, 14, 28, and 42. Bleeds and preparation of EDTA plasma was done 6 days before immunization (pre-immune plasma) and on days 21, 35, and 50 after first immunization. Vaccine responses were monitored by measuring selectivity of plasma antibodies from immunized mice by ELISAs using immobilized FL TDP-43 and target-specific phospho- and non-phospho peptides. Mice were selected for high and specific response to the phospho-site of interest and vaccinated prior to myeloma fusion with three daily booster injections i.p. of the vaccine antigen without adjuvant present.

### Generation of Hybridomas and Selection for Subcloning

Mice were euthanized and fusion with myeloma cells was performed using splenocytes from mice. Screening for antibodies from the successfully fused hybridoma cell lines were performed as follows. Diluted (1:32) cell culture supernatants were analyzed using either ELISAs or Luminex bead-based multiplex assays (Luminex, The Netherlands).

Viable hybridomas were cultured using serum-containing selection media. Hybridomas with preferential binding to TDP-43 inclusions in human FTD brain and clones binding to the different regions of TDP-43 were selected for further subcloning. Following limiting dilution, the clonal hybridomas were grown in low immunoglobulin containing medium and stable colonies were selected for antibody screening, selection, sequencing, and expression as recombinant antibodies on murine IgG2a backbone for further characterization, selection, and assay development. For all assays shown, antibodies were used either as a recombinant preparation or purified from hybridoma supernatant.

### Example 2: Antibody Sequencing

For all sequences originating from mouse hybridoma clones, cell lysates were used for gene sequencing of the variable regions. Hybridomas were grown, harvested, and lysed using a lysis buffer containing guanidinium salts to deactivate Rnases. Genomic DNA was then eliminated by Rnase-free Dnase, and RNA was purified with a silica-based affinity column using multiple washes and eluted from the column using Rnase-free water. Once the RNA was extracted, its purity and concentration were measured spectrophotometrically. The integrity of the RNA was assessed on a denaturing agarose gel and RNA was reverse transcribed into cDNA using reverse transcriptase (RT). Before adding the RT reaction mixture, the RNA was heated to 70° C. for 10 min to disrupt RNA secondary structures. The RT products were directly used for PCR amplification. For high-fidelity PCR amplification of the cDNA, each of the variable region primers corresponding to the different gene families encoding for antibodies were individually mixed with primers for VH and VL chains separately in a total reaction volume of 50 L. Initially, a degenerate primer pool was used consisting of 12 for VH and 12 for VL, and depending on the results, a second pool was used to obtain PCR products. After the PCR reaction, the products were analyzed by gel electrophoresis using 2% agarose gels and stained with ethidium bromide to visualize the bands. The PCR products for VL and VH were individually purified on an agarose gel using tris-acetate-EDTA (TAE). The purified fragments excised from the gel were sequenced with the dye-terminator sequencing method using the same primers as those used for the PCR. Sequencing was carried out in both directions to provide overlap at both ends. Sequences were then analyzed using multiple sequence alignment (Clustal tool) and annotated using the algorithm as described by Kabat et al., Sequences of Proteins of Immunological Interest, 91-3242 (1991). Nucleotide sequences of the heavy chain and light chain variable domains (VH and VL) are shown in Table 2. Translated protein sequences for selected VH and VL chain variable domains, and their complementarity-determining regions (CDRs) are shown in Table 3.

TABLE 2
Nucleotide sequences of the heavy chain and light chain variable domains (VH and VL)

| Antibody | Hybridoma | | |
|---|---|---|---|
| Name | Code | VH | VL |
| ACI-8071- | 943.12A8B4 | GAGGTGCAGCTTGTTGAGTCTGGTGGAG | GATGTTGTGATGACCCAGACTCCACTCAC |
| 943.12A8- | | GATTGGTGCACCCTAAAGGATCATTGCA | TTTGTCGGTTACCATTGGACAGCCAGCCT |
| Ab1 | | ACTCTCATGTGCCGCCTCTGGTTTCACC | CCATCTCTTGCAGGTCAAGTCAGAGCCTC |
| | | TTCAATACCTATACCATGCACTGGGTCC | TTAGATCGTGATGGAGAGACATATTTGAA |
| | | GCCAGGCTCCAGGAAAGGGTTTGGAAT | TTGGTTGTTACAGAGGCCAGGCCAGTCTC |
| | | GGGTTGCTCGCATAAGAAGTAAAAGGA | CAAAGCGCCTAATCTATCTGGTGTCTAAA |
| | | GTAATTATGCAACATATTATGCCGATTC | CTGGACTCTGGAGTCCCTGACAGGTTCAC |
| | | AGTGAAAGACAGATTCACCATCTCCAG | TGGCAGTGGATCAGGGACAGATTTCACAC |
| | | AGATGATTCACAAAGCATGCTCTATCTG | TGAAAATCAGCAGAGTGGAGGCTGAGGA |
| | | CAAATGAACAACCTGAAAACTGAGGAC | TTTGGGAGTTTATTATTGCTGGGAAGGTA |
| | | ACAGCCATATATTACTGTGTGAGAGGCA | CACATCTTCCATTCGCGTTCGGCTCGGGG |
| | | CGGGAAGTTACTGGGGTCAAGGAACCT | ACAAAGTTGGAAATAAAA |
| | | CAGTCACCGTCTCCTCA | (SEQ ID NO: 19) |
| | | (SEQ ID NO: 18) | |
| ACI-8071- | 943.7H9H8 | GAAATTCAGCTCCAGCAGTCTGGGACTG | GATGTTGTGATGACCCAAATTCCACTCTC |
| 943.7H9-Ab1 | | TGATGGCAAGGCCTGGGGCTTCAGTGA | CCTGCCTGTCAGTCTTGGAGATCAAGCCT |
| | | AGATGTCCTGCAAGACTTCTGGCTACAT | CCATCTCTTGCAGATCTAGTCAGAGACTT |
| | | ATTTACCAACTACTGGATGCACTGGGTA | GTACACAGTAATGGAAACACCTATTTACA |
| | | AAACAGAGGCCTGGACAGGGTCTGGAC | TTGGTACCTGCAGAAGCCAGGCCAGTCTC |
| | | TGGATAGGGACTATTTATCCTGGAAATA | CAAAGTTCCTGATCCACAAAGTTTCCAAC |
| | | GTGATACTGACTACAACCAGAACTTTAA | CGATTTTCTGGGGTCCCAGACAGGTTCAG |
| | | GGGCAAGGCCAAACTGACTGCAGTCAC | TGGCAGTGGATCAGGGACAGATTTCACAC |
| | | ATCCGCCAGCACTGCCTACATGGAGCTC | TCAAGATCAGAAGAGTGGAGGCTGAGGA |
| | | AGCAGCCTGACAAATGAGGACTCTGCG | TCTGGGAGTATATTTCTGCTGTCAAAGTA |
| | | GTCTATTTCTGTATAAGAGGGGGATGGG | CACATGTTCCGTACACGTTCGGAGGGGGG |
| | | GAGGGTTTCCTTACTGGGGCCAAGGGA | ACCAAGCTGGAAATAAAA |
| | | CTCTGGTCACTGTCTCTGCA | (SEQ ID NO: 29) |
| | | (SEQ ID NO: 28) | |
| ACI-8071- | 943.7D3E2 | CAGGTCCAATTACAGCAGCCTGGGACT | GATGTTGTGATGACCCAGACTCCACTCAT |
| 943.7D3-Ab1 | | GAACTGGTGAAGCCTGGGGCTTCAGTG | TTTGTCGGTTACCATTGGACAACCAGCCT |
| | | AAACTGTCCTGCAAGGCTTCTGGCTACA | CCATCTCTTGCAAGTCAGGTCAGAGCCTC |
| | | CCTTCACCAGGTACTGGATGCACTGGAT | TTAGATAGTGATGGATGGACATATCTTAA |
| | | GAAACAGAGGCCAGGACAAGGCCTTGA | TTGGATGTTCCAGCGGCCAGGCCAGTCTC |
| | | GTGGATTGGAAATATTAATCCTAGCGAT | CAAAGCGCCTAATCTATCTGGTGTCTAAA |
| | | GGTGGTACCAACTACAATGAGAAGTTC | CTGGACTCTGGAGTCCCTGACAGGTTCAC |
| | | AAGAATAAGGCCTCACTGACTGTAGAC | TGGCAGTGGATCAGGGACAGATTTCACAC |
| | | AAATCCTCCAGTACAGCCTACATGCAGC | TGAAGATCAGCAGAGTGGAGGCTGAGGA |
| | | TCAGCAGGCTGACATCTGAGGACTCGG | TTTGGGAGTTTATCATTGCTGGCAAGGTA |
| | | CGGTCTATTATTGTGCAAGACGCGGCTC | CACATCTTCCGTACACGTTCGGAGGGGGG |
| | | GGGCTACTGGGGCCAAGGCACCACTCT | ACCACGCTGGAAATAAAA |
| | | CACAGTCTCCTCA | (SEQ ID NO: 39) |
| | | (SEQ ID NO: 38) | |
| ACI-8071- | 943.2E6E10 | CAGGTCCAACTGCAGCAGCCTGGGACT | GATGTTGTGATGACCCAGACTCCACTCAC |
| 943.2E6-Ab1 | | GAACTGGTGAAGCCTGGGGCTTCAGTG | TTTGTCGGTTACCATTGGACAACCAGCCT |
| | | AAGCTGTCCTGCAAGGCTTCTGGCTACA | TCATCTCTTGCAAGTCAAGTCAGAGCCTC |
| | | CCTTCACCAGCTACTGGATGCACTGGGT | TTAGATAGTAATGGAAAGACATATCTGAA |
| | | GAAGCAGAGGCCTGGACAAGGCCTTGA | TTGGATGTTACAGAGGCCAGGCCAGTCTC |
| | | GTGGATTGGAAATATTAATCCTATCAAT | CAAAGCGCCTAATCTATCTGGTGGCTAAA |
| | | AGTGATACTAACTACAATGAGAAGTTC | CTGGACTCTGGAGTCCCTGACAGGCTCAC |
| | | AAGACCAAGGCCACACTGACTGTAGAC | TGGCAGCGGATCAGGGACAGATTACACA |
| | | AAATCCTCCAGCACAGCCTACATGCAGC | CTGAAAATCAGCAGAGTGGAGGCTGAGG |
| | | TCAGCAGCCTGACATCTGAGGACTCTGC | ATTTGGGAGTTTATTATTGCTGGCAAGGT |
| | | GGTCTATTATTGTGCAAGACGCGGCTCG | ACACATATTCCGTACACGTTCGGAGGGGG |
| | | GGCTACTGGGGCCAAGGCACCACTCTC | GACCAAGCTGGAAATAAAA |
| | | ACAGTCTCCTCA | (SEQ ID NO: 49) |
| | | (SEQ ID NO: 48) | |
| ACI-8072- | 946.8H6D10 | GAGGAGCACCTGGTGGAGTCTGGGGGA | GATGTTTTGATGACCCAGACTCCACTCTC |
| 946.8H6-Ab1 | | GGCTTAGTGAAGCCTGGAGGGTCCCTG | CCTGCCTGTCAGTCTTGGAGATCAAGCCT |
| | | AAACTCTCCTGTGCAGCCTCTGGATTCA | CCATCTCTTGCAGATCTAGTCAGAGTATT |
| | | CTTTCAGTGACTATGGAATGCAGTGGGT | GTCCATAGTAATGGAAACACCTATTTAGA |
| | | TCGTCAGGCTCCAGAGAAGGGGCTGGA | ATGGTACCTGCAGAAACCTGGCCAGTCTC |
| | | GTGGGTTGCATACATTAGTAGTGGCAGT | CAAAGCTCCTGATCTACAAAGTTTCCAAT |
| | | AGTACCATCTACTATGAAGACACAGTG | CGATTTTCTGGGGTCCCAGACAGGTTCAG |
| | | AAGGGCCGATTCACCATCTCCAGAGAC | TGGCAGTGGATCAGGGACAGATTTCACAC |
| | | AATGCCAAGAACACCCTGTTCCTGCAAA | TGAAGATCAGTAGAGTGGAGGCTGAGGA |
| | | TGACCAGTCTGAGGTCTGAGGACACGG | TGTGGGAGTTTATTACTGCTTTCAAGGTT |
| | | CCATGTACTACTGTGTAAGGCCCTATGA | CACATGTTCCTCGGACGTTCGGGGGAGGC |
| | | TAACTACGGCCAAGGGACTCTGGTCACT | ACCAAGCTGGAAATCAAA |
| | | GTCTCTGCA | (SEQ ID NO: 59) |
| | | (SEQ ID NO: 58) | |
| ACI-8072- | 946.4G5F1 | GAGGTGCAGCTGGTGGAGTCTGGGGGA | GATGTTTTGATGACCCAAACTCCACTCTC |
| 946.4G5-Ab1 | | GGCTTAGTGAAGCCTGGAGGGTCCCTG | CCTGCCTGTCAGTCTTGGAGATCAAGCCT |
| | | AAACTCTCCTGTGCAGCCTCTGGATTCA | CCATCTCTTGCAGATCTAGTCAGAGCATT |
| | | CTTTCAGTGACTATGGAATGCACTGGGT | GTACATAGTAATGGAAACACCTATTTAGA |
| | | TCGTCAGGCTCCAGAGAAGGGGCTGGA | ATGGTACCTGCAGAAACCAGGCCAGTCTC |
| | | GTGGGTTGCATACATTAGTAGTGGCAGT | CAAAGCTCCTGATCTACAAAGTTTCCAAC |
| | | AGTACCATCTACTATGCAGACACAGTGA | CGATTTTCTGGGGTCCCAGACAGGTTCAG |
| | | AGGGCCGATTCACCATGTCCAGAGACA | TGGCAGTGGATCAGGGACAGATTTCACAC |
| | | ATGCCAAGAACACCCTGTTCCTGCAAAT | TCAAGATCAGCAGAGTGGAGGCTGAGGA |
| | | GACCAGTCTGAGGTCTGAGGACACGGC | TCTGGGAGTTTATTACTGCTTTCAAGGTTC |
| | | CATGTATTACTGTTCAAGGCCCTATGGA | ACATGTTCCTCGGACGTTCGGTGGAGGCA |
| | | AACTACGGCCAAGGGACTCTGGTCACT | CCAAGCTGGAAATCAAA |
| | | GTCTCTGCA | (SEQ ID NO: 69) |
| | | (SEQ ID NO: 68) | |
| ACI-8072- | 946.9D6F3 | GAGGAGCACCTGGTGGAGTCTGGGGGA | GACATTGTGCTGACACAGTCTCCTGCTTC |
| 946.9D6-Ab1 | | GGCTTAGTGAAGCCTGGAGGGTCCCTG | CTTAGCTGTATCTCTGGGGCAGAGGGCCA |
| | | AAACTCTCCTGTGCAGCCTCTGGATTCA | CCATCTCATGCAGGGCCAGCAAAAGTGTC |
| | | CTTTCAGTGACTATGGAATGCAGTGGGT | AGTACATCTGGCTATAGTTATATGCACTG |
| | | TCGTCAGGCTCCAGAGAAGGGGCTGGA | GTACCAACAGAAACCAGGACAGCCACCC |
| | | GTGGGTTGCATACATTAGTAGTGGCAGT | AAACTCCTCATCTATCTTGCATCCAACCT |
| | | AGTACCATCTACTATGAAGACACAGTG | AGAATCTGGGGTCCCTGCCAGGTTCAGTG |
| | | AAGGGCCGATTCACCATCTCCAGAGAC | GCAGTGGGTCTGGGACAGACTTCACCCTC |
| | | AATGCCAAGAACACCCTGTTCCTGCAAA | AACATCCATCCTGTGGAGGAGGAGGATG |
| | | TGACCAGTCTGAGGTCTGAGGACACGG | CTGCAACCTATTACTGTCAGCACAGTAGG |
| | | CCATGTACTACTGTGTAAGGCCCTATGA | GAGCTTCCGTGGACGTTCGGTGGAGGCAC |
| | | TAACTACGGCCAAGGGACTCTGGTCACT | CAAGCTGGAAATCAAA |
| | | GTCTCTGCA | (SEQ ID NO: 79) |
| | | (SEQ ID NO: 58) | |
| ACI-7071- | 4665-B5- | CAGGTGCAGCTGAAGCAGTCTGGACCT | GATGTTTTGATGACCCAAACTCCACTCAC |
| 4665-B5- | R3B-B8 | GAGCTGGTGAAGCCTGGGGCTTCAGTG | TTTGTCGGTTACCATTGGACAACCAGCCT |
| R3B-Ab2 | | AAGTTGTCCTGCAAGGCTTCTGGCTACA | CCATCTCTTGCAAGTCAAGTCAGAGCCTC |
| | | CCTTCACAACCTACGATATAAACTGGGT | TTAGATAGTGATGGAAAGACATATTTGAA |
| | | GAAGCAGAGGCCTGGACAGGGACCTGA | TTGGTTGTTTCAGAGGCCAGGCCAGTCTC |
| | | GTGGATTGGATGGATTTATCCTAGAGTT | CAAAGCGCCTAATCTATCTGGTGTCTAAA |
| | | GGTAATACTAAGTACAATGAGAAGTTC | CTGGACTCTGGAGTCCCTGACAGGTTCAC |
| | | AAGGACAAGGCCACATTGACTGTAGAC | TGGCAGTGGATCAGGGACAGATTTCACAC |
| | | ACATCCTCCAGCACAGCGTACATGGAG | TGAAAATCAGCAGAGTGGAGGCTGAGGA |
| | | CTCCACAGCCTGACATCTGAGGACTCTG | TTTGGGAGTTTATTACTGCTTTCAAGGTTC |
| | | CGGTCTATTTCTGTGCAAGCGCGTTGAC | ACATGTTCCTCTCACGTTCGGTGCTGGGA |
| | | CTACTGGGGCCAAGGGACTCTGGTCACT | CCAAGCTGGAGCTGAAA |
| | | GTCTCTGCA | (SEQ ID NO: 89) |
| | | (SEQ ID NO: 88) | |
| ACI-7069- | 636E5B8 | GAGGTACATCTGGTGGAGTCTGGGGGA | CAACTTGTGCTCACTCAGTCATCTTCAGC |
| 636E5-Ab1 | | GACTTAGTGATGCCTGGAGGGTCCCTGA | CTCTTTCTCCCTGGGAGCCTCAGCAAAAC |
| as described | | AGCTCTCCTGTGCAGCCTCTGGATTCAC | TCACGTGCACCTTGAGTAGTCAGCACAGT |
| in | | TTTCAGTAACTATGGCATGTCTTGGGTT | ACGTACACCATTGAATGGTATCAGCAACA |
| WO2020234473 | | CGCCAGACTCCAGACAAGAGGCTGGAG | GCCACTCAAGCCTCCTAAGTATGTGATGG |
| | | TGGGTCGCAACCATTAGTAGTGGTGGTA | AGCTTAAGAAAGATGGAAGCCACAGCAC |
| | | AATATATCAACTACTTAGACAGTTTGAA | AGGTGATGGGATTCCTGATCGCTTCTCTG |
| | | GGGGCGATTCACCATCTCCAGAGACAA | GATCCAGCTCTGGTGCTGATCGCTACCTT |
| | | TGCCAAGAACACCCTATACCTGCAAATG | AGCATTTCCAACATCCAGCCTGAAGATGA |
| | | AGCAGTCTGAAGTCTGAGGATACAGCC | AGCAATATACATCTGTGGTGTGGGTGATA |
| | | ATGTATTACTGTGCAAAAGACTACGGTA | CAATTAAGGAACAATTTGTGTATGTTTTC |
| | | GTGGCTGGGCCTGGTTTGCTTACTGGGG | GGCGGTGGAACCAAGGTCACTGTCCTA |
| | | CCAAGGGACTCTGGTCACTGTCTCTGCA | (SEQ ID NO: 99) |
| | | (SEQ ID NO: 98) | |
| ACI-7071- | 809F12D8 | CAGGTGCAGCTGAAGGAGTCAGGACCT | GATGTTTTGATGACCCAAACTCCACTCTC |
| 809F12-Ab1 | | GGCCTGGTGGCGCCCTCACAGAGCCTGT | CCTGCCTGTCAGTCTTGGAGATCAGGCCT |
| as described | | CCATCACTTGCACTGTCTCTGGGTTTTC | CCATCTCTTGCAGATCTAGTCAGAACATT |
| in | | GTTAAACAGAAATGGTGTACAGTGGGT | GTACATAGTATTGGAAACACCTATTTAGA |
| WO2020234473 | | TCGCCAGCCTCCAGGAAAGGGTCTGGA | GTGGTACCTGCAGAAACCAGGCCAGTCTC |
| | | GTGGCTGGGAGTAATATGGCCTGGCGG | CAAAGCTCCTGATCTACAAAGTTTCCAAC |
| | | AAGCACAAATTGTAATTCGGCTCTCATG | CGATTTTCTGGGGTCCCAGACAGGTTCAG |
| | | TCCAGACTGAGCATCAGCAAAGACAAC | TGGCAGTGGATCAGGGACAGATTTCACAC |
| | | TCCAAGAGTCAAGTTTTCTTAAAAATGA | TCAAGATCAGCAGAGTGGAGGCTGAGGA |
| | | ACAGTCTGCACACTGATGACACAGGCA | TCTGGGAGTTTATTACTGCTTTCAAGGTTC |
| | | TATATTACTGTGCCAGAGTAGGGGGTAA | ACATGTTCCGTACACGTTCGGAGGGGGGA |
| | | CTACGTGTGGGACTATAATAACTACGCC | CCAAGCTAGAAATAAGA |
| | | TGGGGCCAAGGGACTCTGGTCACTGTCT | (SEQ ID NO: 109) |
| | | CTGCA | |
| | | (SEQ ID NO: 108) | |
| ACI-8070- | 942.30D12F9 | GATGTGCAGCTGGTGGAGTCTGGGGGA | GACATCAAGATGACCCAGTCTCCATCTTC |
| 942.30D12- | | GGCTTAGTGCAGCCTGGAGGGTCCCGG | CATGTATGCGTCTCTAGGAGAGAGAGTCA |
| Ab1 | | AAACTCTCCTGTGAAGCCTCTGGATTCA | CTATCACTTGCAAGGCGAGTCAGGACATT |
| | | CTTTCAGTAGCATTGGAATGCACTGGGT | AATAGCTATTTAAGCTGGTTCCAGCAGAA |
| | | TCGTCAGGCTCCAGAGAAGGGGCTGGA | ACCAGGGAAATCTCCTAAGACCCTGATCT |
| | | GTGGGTCGCATATATTACTAGTGGCAGT | ATCGTGCAAACAGATTGGTTGATGGGGTC |
| | | AGTACCATCTACTATGCAGACACAGTGA | CCATCAAGGTTCAGTGGCAGTGGATCTGG |
| | | AGGGCCGATTCACCATCTCCAGAGACA | GCAAGATTATTCTCTCACCATCAGCAGCC |
| | | ATCCCAAGAACACCCTGTTCCTGCAAAT | TGGAGTATGAAGATATGGGAATTTATTAT |
| | | GACCAGTCTAAGGTCTGAGGACACGGC | TGTCTACAGTATGATGAGTTTCCTCTCAC |
| | | CATCTATTACTGTGCAGGATCTGGACCT | GTTCGGTGCTGGGACCAAGCTGGAGCTGA |
| | | GGGACTGACTACTGGGGCCAAGGCACC | AA |
| | | ACTCTCACAGTCTCCTCA | (SEQ ID NO: 119) |
| | | (SEQ ID NO: 118) | |

TABLE 3
Amino acid sequences of the heavy chain and light chain variable domains (VH and VL) and their CDRs

| Antibody | Hybridoma | | VH | VH | VH | | VL | VL | VL |
|---|---|---|---|---|---|---|---|---|---|
| name | code | VH | CDR1 | CDR2 | CDR3 | VL | CDR1 | CDR2 | CDR3 |
| ACI-8071- | 943.12A8B4 | EVQLVESGGGL | TYTM | RIRSK | GTGS | DVVMTQTPLTLS | RSSQS | LVSK | WEGT |
| 943.12A8- | | VHPKGSLQLSC | H | RSNY | Y (SEQ | VTIGQPASISCRS | LLDRD | LDS | HLPFA |
| Ab1 | | AASGFTFNTYT | (SEQ | ATYY | ID NO: | SQSLLDRDGETY | GETYL | (SEQ | (SEQ |
| | | MHWVRQAPG | ID NO: | ADSV | 13) | LNWLLQRPGQS | N | ID NO: | ID NO: |
| | | KGLEWVARIRS | 11) | KD | | PKRLIYLVSKLD | (SEQ | 16) | 17) |
| | | KRSNYATYYA | | (SEQ | | SGVPDRFTGSGS | ID NO: | | |
| | | DSVKDRFTISR | | ID NO: | | GTDFTLKISRVE | 15) | | |
| | | DDSQSMLYLQ | | 12) | | AEDLGVYYCWE | | | |
| | | MNNLKTEDTAI | | | | GTHLPFAFGSGT | | | |
| | | YYCVRGTGSY | | | | KLEIK | | | |
| | | WGQGTSVTVS | | | | (SEQ ID NO: 14) | | | |
| | | S | | | | | | | |
| | | (SEQ ID NO: 10) | | | | | | | |
| ACI- | 943.7H9H8 | EIQLQQSGTVM | NYW | TIYPG | GGWG | DVVMTQIPLSLP | RSSQR | KVSN | CQSTH |
| 8071- | | ARPGASVKMS | MH | NSDT | GFPY | VSLGDQASISCR | LVHSN | RFS | VPYT |
| 943.7H9- | | CKTSGYIFTNY | (SEQ | DYNQ | (SEQ | SSQRLVHSNGNT | GNTY | (SEQ | (SEQ |
| Ab1 | | WMHWVKQRP | ID NO: | NFKG | ID NO: | YLHWYLQKPGQ | LH | ID NO: | ID NO: |
| | | GQGLDWIGTIY | 21) | (SEQ | 23) | SPKFLIHKVSNR | (SEQ | 26) | 27) |
| | | PGNSDTDYNQ | | ID NO: | | FSGVPDRFSGSG | ID NO: | | |
| | | NFKGKAKLTA | | 22) | | SGTDFTLKIRRV | 25) | | |
| | | VTSASTAYMEL | | | | EAEDLGVYFCC | | | |
| | | SSLTNEDSAVY | | | | QSTHVPYTFGGG | | | |
| | | FCIRGGWGGFP | | | | TKLEIK | | | |
| | | YWGQGTLVTV | | | | (SEQ ID NO: 24) | | | |
| | | SA | | | | | | | |
| | | (SEQ ID NO: 20) | | | | | | | |
| ACI-8071- | 943.7D3E2 | QVQLQQPGTEL | RYWM | NINPS | RGSG | DVVMTQTPLILS | KSGQS | LVSK | WQGT |
| 943.7D3- | | VKPGASVKLSC | H | DGGT | Y | VTIGQPASISCKS | LLDSD | LDS | HLPYT |
| Ab1 | | KASGYTFTRY | (SEQ | NYNE | (SEQ | GQSLLDSDGWT | GWTY | (SEQ | (SEQ |
| | | WMHWMKQRP | ID NO: | KFKN | ID NO: | YLNWMFQRPGQ | LN | ID NO: | ID NO: |
| | | GQGLEWIGNIN | 31) | (SEQ | 33) | SPKRLIYLVSKL | (SEQ | 16) | 37) |
| | | PSDGGTNYNE | | ID NO: | | DSGVPDRFTGSG | ID NO: | | |
| | | KFKNKASLTV | | 32) | | SGTDFTLKISRV | 35) | | |
| | | DKSSSTAYMQ | | | | EAEDLGVYHCW | | | |
| | | LSRLTSEDSAV | | | | QGTHLPYTFGG | | | |
| | | YYCARRGSGY | | | | GTTLEIK | | | |
| | | WGQGTTLTVS | | | | (SEQ ID NO: 34) | | | |
| | | S | | | | | | | |
| | | (SEQ ID NO: 30) | | | | | | | |
| ACI-8071- | 943.2E6E10 | QVQLQQPGTEL | SYWM | NINPI | RGSG | DVVMTQTPLTLS | KSSQS | LVAK | WQGT |
| 943.2E6- | | VKPGASVKLSC | H | NSDT | Y | VTIGQPAFISCKS | LLDSN | LDS | HIPYT |
| Ab1 | | KASGYTFTSY | (SEQ | NYNE | (SEQ | SQSLLDSNGKTY | GKTY | (SEQ | (SEQ |
| | | WMHWVKQRP | ID NO: | KFKT | ID NO: | LNWMLQRPGQS | LN | ID NO: | ID NO: |
| | | GQGLEWIGNIN | 41) | (SEQ | 33) | PKRLIYLVAKLD | (SEQ | 46) | 47) |
| | | PINSDTNYNEK | | ID NO: | | SGVPDRLTGSGS | ID NO: | | |
| | | FKTKATLTVDK | | 42) | | GTDYTLKISRVE | 45) | | |
| | | SSSTAYMQLSS | | | | AEDLGVYYCWQ | | | |
| | | LTSEDSAVYYC | | | | GTHIPYTFGGGT | | | |
| | | ARRGSGYWGQ | | | | KLEIK | | | |
| | | GTTLTVSS | | | | (SEQ ID NO: 44) | | | |
| | | (SEQ ID NO: 40) | | | | | | | |
| ACI-8072- | 946.8H6D10 | EEHLVESGGGL | DYGM | YISSG | PYDN | DVLMTQTPLSLP | RSSQS | KVSN | FQGSH |
| 946.8H6- | | VKPGGSLKLSC | Q | SSTIY | Y | VSLGDQASISCR | IVHSN | RFS | VPRT |
| Ab1 | | AASGFTFSDYG | (SEQ | YEDT | (SEQ | SSQSIVHSNGNT | GNTY | (SEQ | (SEQ |
| | | MQWVRQAPEK | ID NO: | VKG | ID NO: | YLEWYLQKPGQ | LE | ID NO: | ID NO: |
| | | GLEWVAYISSG | 51) | (SEQ | 53) | SPKLLIYKVSNR | (SEQ | 26) | 57) |
| | | SSTIYYEDTVK | | ID NO: | | FSGVPDRESGSG | ID NO: | | |
| | | GRFTISRDNAK | | 52) | | SGTDFTLKISRV | 55) | | |
| | | NTLFLQMTSLR | | | | EAEDVGVYYCF | | | |
| | | SEDTAMYYCV | | | | QGSHVPRTFGG | | | |
| | | RPYDNYGQGT | | | | GTKLEIK | | | |
| | | LVTVSA | | | | (SEQ ID NO: 54) | | | |
| | | (SEQ ID NO: 50) | | | | | | | |
| ACI-8072- | 946.4G5F1 | EVQLVESGGGL | DYGM | YISSG | PYGN | DVLMTQTPLSLP | RSSQS | KVSN | FQGSH |
| 946.4G5- | | VKPGGSLKLSC | H | SSTIY | Y | VSLGDQASISCR | IVHSN | RFS | VPRT |
| Ab1 | | AASGFTFSDYG | (SEQ | YADT | (SEQ | SSQSIVHSNGNT | GNTY | (SEQ | (SEQ |
| | | MHWVRQAPEK | ID NO: | VKG | ID NO: | YLEWYLQKPGQ | LE | ID NO: | ID NO: |
| | | GLEWVAYISSG | 61) | (SEQ | 63) | SPKLLIYKVSNR | (SEQ | 26) | 57) |
| | | SSTIYYADTVK | | ID NO: | | FSGVPDRFSGSG | ID NO: | | |
| | | GRFTMSRDNA | | 62) | | SGTDFTLKISRV | 55) | | |
| | | KNTLFLQMTSL | | | | EAEDLGVYYCF | | | |
| | | RSEDTAMYYC | | | | QGSHVPRTFGG | | | |
| | | SRPYGNYGQG | | | | GTKLEIK | | | |
| | | TLVTVSA | | | | (SEQ ID NO: 64) | | | |
| | | (SEQ ID NO: 60) | | | | | | | |
| ACI-8072- | 946.9D6F3 | EEHLVESGGGL | DYGM | YISSG | PYDN | DIVLTQSPASLA | RASKS | LASN | QHSRE |
| 946.9D6- | | VKPGGSLKLSC | Q | SSTIY | Y | VSLGQRATISCR | VSTSG | LES | LPWT |
| Ab1 | | AASGFTFSDYG | (SEQ | YEDT | (SEQ | ASKSVSTSGYSY | YSYM | (SEQ | (SEQ |
| | | MQWVRQAPEK | ID NO: | VKG | ID NO: | MHWYQQKPGQ | H | ID NO: | ID NO: |
| | | GLEWVAYISSG | 51) | (SEQ | 53) | PPKLLIYLASNLE | (SEQ | 76) | 77) |
| | | SSTIYYEDTVK | | ID NO: | | SGVPARFSGSGS | ID NO: | | |
| | | GRFTISRDNAK | | 52) | | GTDFTLNIHPVE | 75) | | |
| | | NTLFLQMTSLR | | | | EEDAATYYCQH | | | |
| | | SEDTAMYYCV | | | | SRELPWTFGGGT | | | |
| | | RPYDNYGQGT | | | | KLEIK | | | |
| | | LVTVSA | | | | (SEQ ID NO: 74) | | | |
| | | (SEQ ID NO: 50) | | | | | | | |
| ACI-7071- | 4665-B5- | QVQLKQSGPEL | TYDIN | WIYPR | ALTY | DVLMTQTPLTLS | KSSQS | LVSK | FQGSH |
| 4665-B5- | R3B-B8 | VKPGASVKLSC | (SEQ | VGNT | (SEQ | VTIGQPASISCKS | LLDSD | LDS | VPLT |
| R3B-Ab2 | | KASGYTFTTYD | ID NO: | KYNE | ID NO: | SQSLLDSDGKTY | GKTY | (SEQ | (SEQ |
| | | INWVKQRPGQ | 81) | KFKD | 83) | LNWLFQRPGQSP | LN | ID NO: | ID NO: |
| | | GPEWIGWIYPR | | (SEQ | | KRLIYLVSKLDS | (SEQ | 16) | 87) |
| | | VGNTKYNEKF | | ID NO: | | GVPDRFTGSGSG | ID NO: | | |
| | | KDKATLTVDTS | | 82) | | TDFTLKISRVEA | 85) | | |
| | | SSTAYMELHSL | | | | EDLGVYYCFQG | | | |
| | | TSEDSAVYFCA | | | | SHVPLTFGAGTK | | | |
| | | SALTYWGQGT | | | | LELK | | | |
| | | LVTVSA | | | | (SEQ ID NO: 84) | | | |
| | | (SEQ ID NO: 80) | | | | | | | |
| ACI-7069- | 636E5B8 | EVHLVESGGDL | NYGM | TISSG | DYGS | QLVLTQSSSASF | TLSSQ | GSHST | GVGD |
| 636E5-Ab1 | | VMPGGSLKLSC | S | GKYIN | GWA | SLGASAKLTCTL | HSTYT | GD | TIKEQ |
| as | | AASGFTFSNYG | (SEQ | YLDSL | WFAY | SSQHSTYTIEWY | IE | (SEQ | FVYV |
| described | | MSWVRQTPDK | ID NO: | KG | (SEQ | QQQPLKPPKYV | (SEQ | ID NO: | (SEQ |
| in | | RLEWVATISSG | | (SEQ | ID NO: | MELKKDGSHST | ID NO: | 96) | ID NO: |
| WO2020234473 | | GKYINYLDSLK | 91) | ID | 93) | GDGIPDRESGSSS | 95) | | 97) |
| | | GRFTISRDNAK | | NO:92) | | GADRYLSISNIQP | | | |
| | | NTLYLQMSSLK | | | | EDEAIYICGVGD | | | |
| | | SEDTAMYYCA | | | | TIKEQFVYVFGG | | | |
| | | KDYGSGWAWF | | | | GTKVTVL | | | |
| | | AYWGQGTLVT | | | | (SEQ ID NO: 94) | | | |
| | | VSA | | | | | | | |
| | | (SEQ ID NO: 90) | | | | | | | |
| ACI-7071- | 809F12D8 | QVQLKESGPGL | RNGV | VIWP | VGGN | DVLMTQTPLSLP | RSSQN | KVSN | FQGSH |
| 809F12-Ab1 | | VAPSQSLSITCT | Q | GGST | YVWD | VSLGDQASISCR | IVHSI | RFS | VPYT |
| as | | VSGFSLNRNGV | (SEQ | NCNS | YNNY | SSQNIVHSIGNT | GNTY | (SEQ | (SEQ |
| described | | QWVRQPPGKG | ID NO: | ALMS | A | YLEWYLQKPGQ | LE | ID NO: | ID |
| in | | LEWLGVIWPG | 101) | (SEQ | (SEQ | SPKLLIYKVSNR | (SEQ | 26) | NO: 107) |
| WO2020234473 | | GSTNCNSALMS | | ID NO: | ID NO: | FSGVPDRFSGSG | ID NO: | | |
| | | RLSISKDNSKS | | 102) | 103) | SGTDFTLKISRV | 105) | | |
| | | QVFLKMNSLH | | | | EAEDLGVYYCF | | | |
| | | TDDTGIYYCAR | | | | QGSHVPYTFGG | | | |
| | | VGGNYVWDY | | | | GTKLEIR | | | |
| | | NNYAWGQGTL | | | | (SEQ ID NO: 104) | | | |
| | | VTVSA | | | | | | | |
| | | (SEQ ID NO: 100) | | | | | | | |
| ACI-8070- | 942.30D12F9 | DVQLVESGGG | SIGMH | YITSG | SGPGT | DIKMTQSPSSMY | KASQ | RANR | LQYD |
| 942.30D12- | | LVQPGGSRKLS | (SEQ | SSTIY | DY | ASLGERVTITCK | DINSY | LVD | EFPLT |
| Ab1 | | CEASGFTFSSIG | ID NO: | YADT | (SEQ | ASQDINSYLSWF | LS | (SEQ | (SEQ |
| | | MHWVRQAPEK | 111) | VKG | ID NO: | QQKPGKSPKTLI | (SEQ | ID NO: | ID NO: |
| | | GLEWVAYITSG | | (SEQ | 113) | YRANRLVDGVP | ID NO: | 116) | 117) |
| | | SSTIYYADTVK | | ID NO: | | SRFSGSGSGQDY | 115) | | |
| | | GRFTISRDNPK | | 112) | | SLTISSLEYEDM | | | |
| | | NTLFLQMTSLR | | | | GIYYCLQYDEFP | | | |
| | | SEDTAIYYCAG | | | | LTFGAGTKLELK | | | |
| | | SGPGTDYWGQ | | | | (SEQ ID NO: 114) | | | |
| | | GTTLTVSS | | | | | | | |
| | | (SEQ ID NO: 110) | | | | | | | |

### Example 3: Determination of Target Binding by Immunoassay

The capacity of antibodies to bind phospho TDP-43 and TDP-43 was determined using a sandwich enzyme-linked immunosorbent assay (ELISA), where each antibody was paired with a second TDP-43 antibody previously characterized for TDP-43 binding and performance in sandwich immunoassays, ACI-7071-809F12-Ab1-rec2 (as described in WO2020234473) or ACI-7069-636E5-Ab1 (as described in WO2020234473). ELISA plates (Nunc™ Edge™ 96-Well, Non-Treated, Flat-Bottom Microplate, ThermoFisher Scientific, Denmark) were coated with 5 μg/mL of anti-phospho TDP-43 or anti-TDP-43 antibodies in carbo/bicarbonate buffer, overnight at 4° C. Plates were then washed with 0.05% Tween-20/PBS and blocked with 1% bovine serum albumin (BSA) in 0.05% Tween-20/PBS for 1 hour at 37° C. The plates were then washed again as described above.

Serial-dilutions of target peptide or protein were then added to the plates. For assays with anti-phospho TDP-43 antibodies, one of four peptide conjugates (peptides 11, 23, 28, or 29) each composed of two TDP-43 sequences, one phosphorylated and one non-phosphorylated, conjugated together with a Trioxatridecan-succinamic acid linker (Ttds, binding regions and sequences shown in Table 4) were used. Peptides 11, 23, 28, and 29 were added in a 10-fold serial dilution starting at 0.5 μM. For assays with TDP-43 antibodies only, recombinant full length TDP-43 (SEQ ID NO: 1, FL TDP-43) was diluted 3-fold from 21.9 pM. Plates were incubated for 1 hour at 37° C., and then washed. Two g/mL PEG-biotinylated phospho TDP-43 or TDP-43 antibodies were then respectively added to the wells and incubated for 1 hour at 37° C., after which the plates were washed. Horseradish peroxidase (HRP)-conjugated streptavidin (R&D Systems, Switzerland) was added at 1/200 dilution in 1% BSA and 0.05% Tween-20/PBS for 45 min at room temperature. After the final wash, plates were incubated with 3,3′,5,5′-Tetramethylbenzidine (TMB; BD Biosciences, Switzerland) on a shaker (350 rpm) for 10 min at room temperature, after which 1.2M hydrogen chloride (HCl; Sigma, Switzerland) was added to the plate. The plate was read at 450 nm using an ELISA plate reader (Tecan, Switzerland).

These ELISAs with serial dilution of a target peptide or protein were used to determine and compare the half maximal effective concentration (EC50) for target binding.

Data were analyzed by plotting the optical density at 450 nm against the logarithms of concentrations of target peptide or protein using a four-parameter logistics (4-PL) fit of calibrators, with a 1/Y2 weighting, using the GraphPad Prism (version 8.4.3) application. The EC50 values for capture and detect are summarized in Table 5. All pS403/pS404 TDP-43 antibodies bound to peptide 28 and all pS409/pS410 TDP-43 antibodies bound to peptide 29, with EC50 values for target capture ranging from 0.070 to 3.318 nM and EC50 values for target detect ranging from 0.249 to 1.959 nM, when antibodies were used in a pairing assay (Table 5).

The pS375/pS379 TDP-43 antibody bound peptides 11 and 23, with EC50 values for target capture of 0.904 nM and EC5v values for target detect of 0.056 nM, when the antibody was used in a pairing assay (Table 5).

For TDP-43 antibodies, the EC5 values for target capture ranged from 0.072 to 1.157 nM and EC50 values for target detect were 0.070 and 0.181 nM, when antibodies were used in a pairing assay with FL TDP-43 as the assay target (Table 5).

TABLE 4
Peptides used as targets or calibrators for phospho TDP-43 antibodies and immunoassays

| | TDP-43 | |
|---|---|---|
| Calibrator | calibrator peptide target sites | Amino acid sequence of peptides and linker |
| Peptide 11 | 355-366 and 370-384 (with pS375 | NNQGNMQREPNQA (SEQ ID NO: 4) linked to |
| | and pS379) | GNNSY{pS}GSN{pS}GAAIG (SEQ ID NO: 5) |
| | | via a Trioxatridecan-succinamic acid linker |
| Peptide 23 | 180-195, 199-213, and 370-384 | SKQSQDEPLRSRKVFVTEDMTEDELREFFSQ |
| | (with pS375 and pS379) | (SEQ ID NO: 6) linked to |
| | | GNNSY{pS}GSN{pS}GAAIG (SEQ ID NO: 5) |
| | | via a Trioxatridecan-succinamic acid linker |
| Peptide 28 | 199-213, 355-366, and 396-409 | TEDMTEDELREFFSQNNQGNMQREPNQA |
| | (with pS403 and pS404) | (SEQ ID NO: 7) linked to |
| | | GFNGGFG{pS}{pS}MDSKS (SEQ ID NO: 8) |
| | | via a Trioxatridecan-succinamic acid linker |
| Peptide 29 | 199-213, 355-366, and 401-413 | TEDMTEDELREFFSQNNQGNMQREPNQA |
| | (with pS409 and pS410) | (SEQ ID NO: 7) linked to |
| | | FGSSMDSK{pS}{pS}GWG (SEQ ID NO: 9) via |
| | | a Trioxatridecan-succinamic acid linker |

TABLE 5
EC50 values as determined by pairing ELISA immunoassay

| | | EC50 for | EC50 for |
|---|---|---|---|
| | Human TDP- | capture | detection |
| Antibody | 43 target site | (nM) | (nM) |
| ACI-8070-942.30D12-Ab1 | pS375/pS379 | 0.904 | 0.056 |
| ACI-8071-943.12A8-Ab1 | pS403/pS404 | 0.293 | 0.249 |
| ACI-8071-943.7H9-Ab1 | pS403/pS404 | 0.070 | 0.313 |
| ACI-8071-943.7D3-Ab1 | pS403/pS404 | 0.170 | 0.181 |
| ACI-8071-943.2E6-Ab1 | pS403/pS404 | 3.318 | 0.563 |
| ACI-8072-946.8H6-Ab1 | pS409/pS410 | 0.111 | 1.122 |
| ACI-8072-946.4G5-Ab1 | pS409/pS410 | 0.196 | 1.959 |
| ACI-7071-4665-B5-R3B-Ab2 | 366-369 | 0.072 | 0.181 |
| ACI-7069-636E5-Ab1 | 358-361 | 1.157 | 0.070 |
| (as described in | | | |
| WO2020234473) | | | |
| ACI-7071-809F12-Ab1-rec2 | 199-213 | 0.181 | NA |
| (as described in | | | |
| WO2020234473) | | | |

Data not available (NA).

### Example 4: Phospho-Site Selectivity of TDP-43 Binding Molecules Demonstrated by Capture ELISA

Phospho TDP-43 antibodies were evaluated and selected for assay development using capture ELISA assays to determine binding specificity and capture properties for TDP-43 and the specific phospho sites using both full-length human TDP-43 protein and phospho peptides representing the C-terminus region of human TDP-43 with phosphorylated serine residues at amino acid 375, 379, 403, 404, 409, or 410. Peptides were biotinylated on the N-terminus for capture detection in ELISA or for immobilization on a chip for affinity measurements using SPR (Biacore 8K, GE Healthcare Life Sciences). Each peptide was single or double phosphorylated on sequence residues representing serine 375 (pS375), serine 379 (pS379), serine 403 (pS403), serine 404 (pS404), serine 409 (pS409), or serine 410 (pS410) sites of the full-length human TDP-43 protein. Also included was a peptide representing the same region (amino acids 366-414 of human TDP-43 (SEQ ID NO:1)) but not phosphorylated.

Briefly, for capture detection in ELISA, wells of 96-well plates were coated with 5 μg/mL goat anti-mouse IgG (Fc gamma fragment specific), in carbonate buffer, overnight at 4° C. Plates were then washed with 0.05% Tween-20/PBS and blocked with 1% bovine serum albumin (BSA) in 0.05% Tween-20/PBS for 1 hour at 37° C. Phospho TDP-43 antibodies were then added at 5 μg/mL and incubated for 2 hours at 37° C. after which the plates were washed as described above. Biotinylated peptides representing each of the phospho sites, or non-phosphorylated, were incubated at 0.5 mM in 0.1% BSA and PBS for 1 hour at 37° C. after which the plates were washed. A streptavidin-HRP-conjugate (R&D Systems, Minnesota, United States) was added at 1/200 dilution in 0.05% Tween-20/1% BSA in PBS for 45 minutes in the dark at ambient temperature. After the final wash, plates were incubated with TMB (Sigma-Aldrich, Switzerland), an HRP substrate solution, and read at 405 nm using an ELISA plate reader (Tecan, Switzerland).

Binding and selectivity results by target capture are indicated in Table 6. Relative binding (signal intensity and number of events), as target capture, is indicated as absent (−), low (+), medium (++), or high (+++).

Antibody ACI-8070-942.30D12-Ab1 bound to phospho peptides 370-384, with pS375 and pS379, or pS379. Antibodies ACI-8071-943.12A8-Ab1 and ACI-8071-943.2E6-Ab1 bound to phospho peptides 396-409, with pS403 and pS404, or pS403, or pS404. Antibodies ACI-8071-943.7H9-Ab1 and ACI-8071-943.7D3-Ab1 bound to phospho peptides 396-409, with pS403 and pS404, or pS403, or pS404, and to phospho peptides 401-413 with pS409 and pS410, or pS410. Antibodies ACI-8072-946.8H6-Ab1, ACI-8072-946.4G5-Ab1, and ACI-8072-946.9D6-Ab1 bound to phospho peptides 401-413 with pS409 and pS410, or pS409, or pS410. None of the phospho TDP-43 antibodies bound to non-phosphorylated peptide 366-414. Antibody ACI-7071-4665-B5-R3B-Ab2 bound only to non-phosphorylated peptide 366-414 as each of the phospho peptides are lacking the binding epitope of this antibody, residues 366-369.

TABLE 6
Binding selectivity for TDP-43 phospho-sites. Numbers refer to amino acid residue sequence
number for full-length human TDP-43 protein consisting of 414 amino acids (SEQ ID NO: 1).

| | Human TDP-43 region and phospho site | | | | | | | | | |
|---|---|---|---|---|---|---|---|---|---|---|
| | 370-384 | | | 396-409 | | | 401-413 | | | 366-414 |
| | (pS375 | | | (pS403 | | | (pS409 | | | (no |
| | and | 370-384 | 370-384 | and | 396-409 | 396-409 | and | 401-413 | 401-413 | phosphor- |
| Antibody | pS379) | (pS375) | (pS379) | pS404) | (pS403) | (pS404) | pS410) | (pS409) | (pS410) | ylation) |
| ACI-8070-942.30D12-Ab1 | +++ | − | +++ | − | − | − | − | − | − | − |
| ACI-8071-943.12A8-Ab1 | − | − | − | +++ | ++ | + | − | − | − | − |
| ACI-8071-943.7H9-Ab1 | − | − | − | +++ | +++ | +++ | ++ | − | + | − |
| ACI-8071-943.7D3-Ab1 | − | − | − | +++ | +++ | +++ | ++ | − | + | − |
| ACI-8071-943.2E6-Ab1 | − | − | − | +++ | ++ | + | − | − | − | − |
| ACI-8072-946.8H6-Ab1 | − | − | − | − | − | − | +++ | ++ | +++ | − |
| ACI-8072-946.4G5-Ab1 | − | − | − | − | − | − | +++ | +++ | +++ | − |
| ACI-8072-946.9D6-Ab1* | − | − | − | NA | NA | NA | +++ | +++ | +++ | − |
| ACI-7071-4665-B5-R3B- | NA | NA | NA | − | − | − | − | − | − | +++ |
| Ab2 | | | | | | | | | | |

Relative binding indicated as absent (−), low (+), medium (++), or high (+++). Data generated using hybridoma (*). Data not available (NA).

### Example 5: Affinity Measurements of TDP-43 Binding Molecules Using Surface Plasmon Resonance

For antibodies ACI-8071-943.12A8-Ab1, ACI-8071-943.7H9-Ab1, ACI-8071-943.7D3-Ab1, ACI-8071-943.2E6-Ab1, ACI-8072-946.8H6-Ab1, ACI-8072-946.4G5-Ab1, ACI-8072-946.9D6-Ab1, and ACI-7071-809F12-Ab1-rec2, affinity measurements were performed by surface plasmon resonance (SPR) on a Biacore 8K instrument (Cytiva, Switzerland), using CM5 Series S sensor chips.

The instrument was primed with running buffer PBS-P+ (10× PBS-P+ diluted to 1× in Milli-Q water) and flow-cells (Fc) 1-2 of channels 1-8 of a CM5 Series S sensor chip (Cytiva, 29149603) were activated with a fresh solution of EDC/NHS (Amine Coupling Kit, BR100633, Cytiva) 1:1 ratio of both reagents at 10 μL/min for 420 sec and goat anti-mouse antibody (Jackson Immuno Research Labs, no 115-005-164) was immobilized at 30 μg/mL in 10 mM sodium acetate pH 5 for 420 sec. Next, all Fc were quenched with 1 M ethanolamine (Cytiva, BR100633) for 420 sec. Non-covalently bound antibodies were removed by three regenerations with 10 mM Glycine-HCl pH 1.5 for 30 sec. Immobilization levels were evaluated following ethanolamine quenching.

Each cycle started with the non-covalent capture of the antibody which were diluted in running buffer to a final concentration of 5 μg/mL and injected for 120 sec with a flow rate of 10 μL/min. Antibodies being measured were captured in channels 1-8, leaving Fc 1 as a blank Fc. Capture levels were evaluated following a 120 sec stabilization period after each antibody injection. ACI-7071-809F12-Ab1-rec2 was used as control, binding to the non-phosphorylated sequences represented in peptides 28 and 29.

Injections of peptides 28 or 29 were performed using single-cycle kinetics with increasing concentrations ranging from 1.2 to 100 nM prepared as a 3-fold serial dilution. Injections were performed with contact times of 300 sec/injection at a flow rate of 30 μL/min. A dissociation phase of 900 sec followed the final injection. Sensor surface was regenerated by two injection of 10 mM Glycine-HCl pH 1.7 at a flow rate of 10 μL/min for 60 sec, followed by a stabilization period of 120 sec. Results obtained from single-cycle kinetics were double-referenced using the blank Fc 1 and buffer cycles and evaluated by Biacore 8K evaluation software (Cytiva). The following kinetic parameters were obtained using the 1:1 binding fit model with variable Reflective Index (RI): association rate constant (ka), dissociation rate constant (kd), affinity constant (KD) and saturation response (Rmax). All parameters (except Rmax) are reported in Table 7.

TABLE 7
Association, dissociation, and affinity constants (ka, kd, and KD, respectively).

| | Peptide 28 | | | Peptide 29 | | |
|---|---|---|---|---|---|---|
| | ka | kd | KD | ka | kd | KD |
| Antibody | (1/Ms) | (1/s) | (nM) | (1/Ms) | (1/s) | (nM) |
| ACI-8071-943.12A8-Ab1 | 1.56E+08 | 1.45E+00 | 9.28 | No detectable affinity measured | | |
| ACI-8071-943.7H9-Ab1 | 1.05E+06 | 2.40E−04 | 0.23 | No detectable affinity measured | | |
| ACI-8071-943.7D3-Ab1 | 8.24E+04 | 4.04E−04 | 4.91 | No detectable affinity measured | | |
| ACI-8071-943.2E6-Ab1 | 7.74E+04 | 9.86E−04 | 12.7 | No detectable affinity measured | | |
| ACI-8072-946.8H6-Ab1 | No detectable affinity measured | | | 1.43E+06 | 4.69E−03 | 3.28 |
| ACI-8072-946.4G5-Ab1 | No detectable affinity measured | | | 4.87E+05 | 8.17E−04 | 1.68 |
| ACI-8072-946.9D6-Ab1* | No detectable affinity measured | | | 3.09E+05 | 7.07E−04 | 2.29 |
| ACI-7071-809F12-Ab1- | 5.14E+05 | 2.31E−03 | 4.50 | 4.90E+05 | 3.43E−03 | 7.00 |
| rec2 | | | | | | |

Data using purified hybridoma supernatant (*).

For antibodies ACI-8070-942.30D12-Ab1 and ACI-7071-809F12-Ab1-rec2 affinity was measured on Biacore 5200 instrument (Cytiva, Switzerland). The instrument was primed with running buffer HBS-P+ (10× HBS-P+ diluted to 1× in Milli-Q water) and flow-cells (Fc) 1-4 of a CM7 Series S sensor chip (Cytiva, 29147020) were activated with a fresh solution of EDC/NHS (Amine Coupling Kit type 2, BR100633, Cytiva) 1:1 ratio of both reagents at 10 μL/min for 420 sec and polyclonal rabbit anti-mouse IgG antibody (Cytiva, 29215281) was immobilized at 30 g/mL in 10 mM sodium acetate pH 5.0 for 420 sec. Next, all Fc were quenched with 1 M ethanolamine (Cytiva, BR100633) for 420 sec. Immobilization levels were evaluated following ethanolamine quenching. A mouse IgG2a isotype control antibody and ACI-8070-942.30D12-Ab1 were captured on Fc 1 and Fc 2, respectively. Antibodies were diluted in 10 mM sodium acetate pH5.5 to a final concentration of 50 μg/mL and injected using the aim immobilization level functionality with a target of 10000 RU. Once maximum capture level was reached, a 300 sec stabilization period was applied followed by an injection of a fresh solution of EDC/NHS (Cytiva, BR100633) at 10 μL/min for 30 sec to crosslink captured antibodies. Next, all Fc were quenched with 1 M ethanolamine (Cytiva, BR100633) for 30 sec. Immobilization levels on Fc 1 and Fc 2 channels were 4519 and 6638 RU, respectively. The instrument was primed with running buffer PBS-P+ and injected at 30 μL/min for 2 h to stabilize the sensor chip surface. Single-cycle kinetics injections were done with increasing concentrations of target peptide, ranging from 12.3 to 1000 nM prepared as a 3-fold serial dilution. Injections were performed with contact times of 300 sec/injection at a flow rate of 30 μL/min. A dissociation phase of 600 sec followed the final injection. Sensor surface was regenerated by injection of 10 mM Glycine-HCl pH 1.7 at a flow rate of 10 μL/min for 30 sec, followed by a stabilization period of 300 sec. Results obtained from single-cycle kinetics were double-referenced using the blank Fc 1 and buffer cycles and evaluated using the Biacore Insight Evaluation software (Cytiva). Sensorgram data were fitted with a 1:1 binding fit model with variable Reflective Index (RI). The kinetic parameters (except Rmax) are reported in Table 8.

TABLE 8
Association, dissociation, and affinity
constants (ka, kd, and KD, respectively).

| | TDP-43 peptide consisting of amino | | |
|---|---|---|---|
| | acid sequence (SEQ ID NO: 5) | | |
| | (comprising pS375 and pS379) | | |
| Antibody | ka (1/Ms) | kd (1/s) | KD (nM) |
| ACI-8070-942.30D12-Ab1 | 2.08E+04 | 4.48E−04 | 21.5 |

| | |
|---|---|
| ACI-7071-809F12-Ab1-rec2 | No detectable affinity measured |

Sensorgram data were fitted with a 1:1 biding fit model using a parameter of global RI. Antibodies with a signal below 5 RU at the highest concentration were considered as non-binders, indicated as no detectable affinity in Table 7 and Table 8. Antibody ACI-8070-942.30D12-Ab1 bound to phospho peptide 370-384 (SEQ ID NO: 5) containing pS375 and pS379 with affinity (KD) of 21.5 nM. Antibodies ACI-8071-943.12A8-Ab1, ACI-8071-943.7H9-Ab1, ACI-8071-943.7D3-Ab1, and ACI-8071-943.2E6-Ab1 bound to Peptide 28 with affinities (KD) ranging from 0.23 to 12.7 nM. Antibodies ACI-8072-946.8H6-Ab1, ACI-8072-946.4G5-Ab1, and ACI-8072-946.9D6-Ab1 bound to Peptide 29 with affinities (KD) ranging from 1.68 to 3.28 nM. Antibody ACI-7071-809F12-Ab1-rec2 bound to Peptide 28 and Peptide 29 (both containing the epitope of ACI-7071-809F12-Ab1-rec2, human TDP-43 residues 199-213) with affinities (KD) of 4.50 and 7.00 nM, respectively.

### Example 6: Avidity Measurements Using SPR

Binding avidity of ACI-7071-4665-B5-R3B-Ab2 to soluble FL TDP-43 was evaluated by determining the KD value using SPR (Biacore T200, GE Healthcare Life Sciences). Recombinant human soluble FL TDP-43 was immobilized on a CM5 Series S sensor chip (GE Healthcare Life Sciences) by amine coupling. Soluble TDP-43 was immobilized at a concentration of 5 μg/ml in 10 mM sodium acetate (pH 4.5) with a flow rate of 5 μl/min for 420 seconds resulting in an immobilization level of 150 RU. To evaluate KD values, antibodies ACI-7071-4665-B5-R3B-Ab2, or a control anti-human TDP-43 antibody, were injected at 3-fold dilutions in PBS-P+ starting from 333 nM and dilute down to 0.15 nM. Antibodies were injected at a flow rate of 50 L/min for 90 seconds contact time and 700 seconds dissociation phase followed by three regenerations with 10 mM glycine-HCl (pH 1.7). For the optimized SPR protocol the antibodies were diluted 3-fold starting from 300 nM and dilute down to 1.2 nM and injected for 300 seconds at 30 μL/min followed by 600 seconds of dissociation. The chip surface was regenerated by one injection with 10 mM glycine-HCl (pH 1.7). Results obtained from binding kinetics were double referenced using a blank flow cell and a buffer cycle, and were evaluated with a global 1:1 fitting model with RI. Antibody ACI-7071-4665-B5-R3B-Ab2 bound to soluble human TDP-43 with a KD of 0.39 nM (ka=4.29E+04 1/Ms and kd=1.66E-05 1/s).

Binding avidity of antibody ACI-8070-942.30D12-Ab1 was performed on a Biacore 8K instrument (Cytiva, Switzerland). Biotinylated TDP-43 phospho peptides 370-384 containing pS375 and pS379, pS375, or pS379 were immobilized in individual channels on a SA Series S sensor chip (Cytiva, BR100531) at a concentration of 190 μg/ml in 10 mM sodium acetate (pH 4.0) with a flow rate of 5 μL/min for 60 seconds resulting in immobilization levels ranging from 68 to 104 RU. Antibody ACI-8070-942.30D12-Ab1, or a control anti-human TDP-43 antibody, were injected in single-cycle kinetics of increasing concentrations ranging from 1.2 to 100 nM prepared as 3-fold serial dilutions in PBS-P+. Injections were performed for 300 sec at 30 μL/min followed by 1800 sec of dissociation. The chip surface was regenerated by one injection with 10 mM glycine-HCl pH 1.7 for 45 sec. Results obtained from binding kinetics were double referenced using a reference flow cell and a buffer cycle, and were evaluated with a global 1:1 fitting model with variable RI by the Biacore Insight Evaluation software. Antibody ACI-8070-942.30D12-Ab1 bound to phospho peptide 370-384 containing pS375 and pS379 with a KD of 0.23 nM, to phospho peptide 370-384 containing pS375 with a KD of 75.5 nM, and to a phospho peptide 370-384 containing pS379 with a KD of 0.28 nM.

### Example 7: Detection of TDP-43 Inclusions in Brain Tissue Sections from FTD Type a Subjects by Immunohistochemistry

Target engagement was evaluated by immunohistochemistry experiments on brain section from patients diagnosed with frontotemporal dementia type A (FTD Type A) with TDP-43 pathology. Human FTD Type A brain tissues were obtained from the University of California San Francisco (UCSF) Neurodegenerative Disease Brain Bank. All material was collected from post-mortem donors from whom a written informed consent for brain autopsy and the use of the material and clinical information for research purposes was obtained by the UCSF Neurodegenerative Disease Brain Bank. The brain samples were cut at 10 m thickness, mounted on microscope slides and stored at −80° C. until used for staining.

Frozen brain sections were first thawed at room temperature, fixed with 4% paraformaldehyde (PFA) at 4° C. for 15 min and washed 3× with phosphate buffered saline (PBS). Tissues were then encircled with a hydrophobic pen to minimize the sample volume and avoid spreading. Blocking was performed with 5% bovine serum albumin (BSA) in PBS with 0.25% TritonX-100 for 1 h at room temperature. The sections were incubated overnight at 4° C. with primary antibodies diluted in 2.5% BSA in PBS with 0.25% TritonX-10×. A commercial anti-phospho TDP-43 antibody used as a positive control was diluted at 1:200. The phospho TPD-43 antibodies selected for characterization were tested at 1 μg/mL. Labeling performed with buffer instead of primary antibody was used as a negative control (for secondary antibody only). The next day, sections were washed 3× with PBS. Alexa Fluor-633-labeled goat anti-rat was used for the positive control and Alexa Fluor-647-labeled goat anti-mouse was used for the anti-phospho antibodies. The sections were incubated with secondary antibodies, diluted at 1:500, in PBS for 30 min at room temperature. After three wash steps with PBS, 0.1% Sudan Black dissolved in 70% ethanol was added to the brain sections for 15 min at room temperature to minimize tissue auto-fluorescence. After three final wash steps in PBS, Prolong Antifade Reagent with DAPI was added and the sections were mounted with coverslips. The brain sections were dried in the dark and imaged with the Panoramic 150 Slide Scanner using the DAPI and Cy3 channel. Exposure times in Cy3 and DAPI channels remained the same for all samples. No staining was observed in the negative control condition.

Staining of TDP-43 inclusions in FTD Type A brain sections is shown in Table 9. Relative signal intensity of inclusion-specific binding and staining is indicated as absent (−), low (+), medium (++), or high (+++).

Antibodies ACI-8071-943.12A8-Ab1, ACI-8071-943.7D3-Ab1, ACI-8072-946.4G5-Ab1, and ACI-8072-946.9D6-Ab1 specifically bound to aggregated TDP-43 cytoplasmic inclusions in FTD Type A pathology (Table 8, first results column). No phospho TDP-43 antibody of the present invention bound to nuclear, non-aggregated, physiological TDP-43 (Table 8, second results column). Antibody ACI-7071-4665-B5-R3B-Ab2, which binds to non-phosphorylated TDP-43, stained cytoplasmic TDP-43 inclusion, and stained nuclear TDP-43 as expected.

TABLE 9
Staining of TDP-43 pathology in brain
tissue from FTD Type A subjects

| | IHC detection of | |
|---|---|---|
| | TDP-43 cytoplasmic | IHC detection of |
| Antibody | inclusions | nuclear TDP-43 |
| ACI-8071-943.12A8-Ab1 | +++ | − |
| ACI-8071-943.7D3-Ab1 | ++ | − |
| ACI-8072-946.4G5-Ab1 | + | − |
| ACI-8072-946.9D6-Ab1* | + | − |
| ACI-7071-4665-B5-R3B-Ab2 | + | + |

Relative staining of TDP-43 pathology indicated as absent (−), low (+), medium (++), or high (+++). Data using purified hybridoma supernatant (*).

### Example 8: Detection and Quantification of TDP-43 in Biofluids

### Example 8A: Immunoassays for Detection and Quantification of TDP-43 in Biofluids with ACI-7071-4665-B5-R3B-Ab2 and ACI-7071-809F12-Ab1-rec2

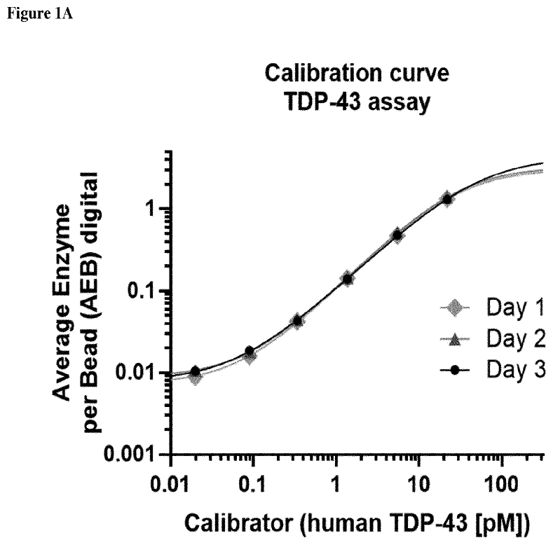

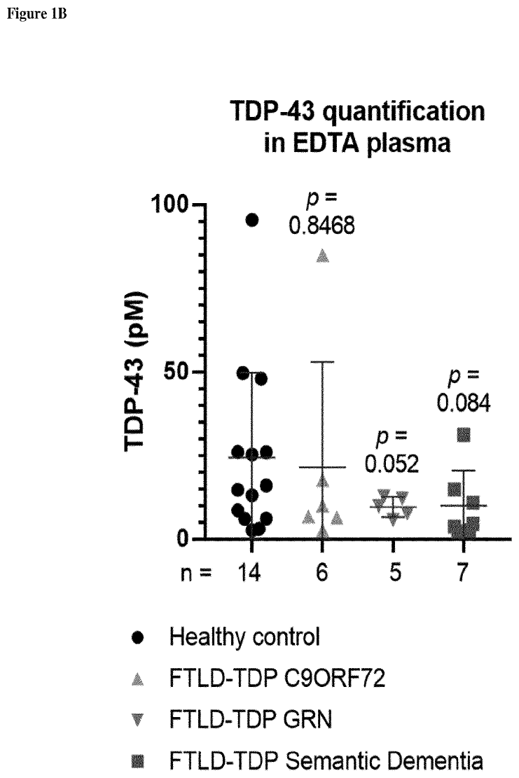

ACI-7071-4665-B5-R3B-Ab2 was used to develop immunoassays to quantify TDP-43 in human plasma. The Single Molecule Array (SIMOA®; Quanterix Corporation, U.S.A.) technology was used to select the best TDP-43 antibodies for the development of high-sensitivity biofluid assays. Briefly, in the SIMOA assay, target-specific antibodies (capture antibody) are coupled to paramagnetic particles (capture reagent). The analyte is captured in semi-homogenous solution and forms an immunocomplex with detection antibodies (detector antibody), capable of generating fluorescent product. At low concentrations, each bead-complex will contain 1 or 0 protein and is trapped into one of 200,000 femtoliter sized microcavities on the array. The digital return signal (on/off) offers enhanced sensitivity over conventional Multiplex ELISA assays. ACI-7071-809F12-Ab1-rec2 was conjugated to paramagnetic beads to serve as capture reagent, and ACI-7071-4665-B5-R3B-Ab2 was biotinylated (#21335 or #21362, ThermoFisher Scientific) to serve as detector antibody. Assays were performed using a three-step protocol with all incubation steps performed at 25° C. on a microplate shaker (Quanterix Corporation) with shaking set at 800 rpm. Samples and calibrators were incubated 30 min with antibody-conjugated capture beads, 10 min with the biotinylated detection antibody, and 10 min with Streptavidin-β-D-Galactosidase (SBG; Quanterix Corporation). The plate was washed between each incubation and at the end of the assay using a magnetic microplate washer (Quanterix Corporation) and read with an SR-X SIMOA® instrument (Quanterix Corporation). Assay optimizations were accomplished by adjusting the concentrations of antibody conjugated to the paramagnetic beads, the concentrations of biotinylated antibody, the molar ratio of biotin and linker used for antibody biotin conjugations, and the concentrations of SBG. Paramagnetic beads conjugated to the capture antibody were diluted in bead diluent (Quanterix Corporation), calibrators and biotinylated antibodies were diluted in a customized diluent specifically formulated for optimum assay performance, and SBG was diluted in SBG diluent (Quanterix Corporation). Following Quanterix SIMOA® assay definition, the lower limit of detection (LLOD) was determined as the mean blank for a readout of average enzyme per bead (AEB)+2.5-times the standard deviation, and corresponded to 0.021 pM. The lower limit of quantification (LLOQ) was set at 0.09 pM, which was the lowest concentration of the calibrator with a pooled CV≤20% and a concentration back calculated from the calibration curve between 80 and 120% of the expected concentration. Analysis of inter-assay variability (FIG. 1A) shows that calibration curves are reproducible. Antibody ACI-7071-4665-B5-R3B-Ab2 can be paired with a second TDP-43 antibody and used to quantify low concentrations of TDP-43 in biofluids, using the SIMOA® immunoassay technology.

To demonstrate the performance of the selected antibodies in measuring TDP-43 in human plasma samples, the assay was further used to quantify TDP-43 in EDTA plasma from healthy control human subjects and from FTLD-TDP (semantic dementia, C9orf72, or GRN) patients. Results suggest a lower plasma TDP-43 concentration in a sub-group of FTLD-TDP patients with a progranulin (GRN) variant or mutation when compared to plasma from healthy controls (FIG. 1B, p=0.052). Values are shown as means±SD, with p values determined using One-way analysis of variance (ANOVA) followed by the Two-stage step-up method of Benjamini, Krieger and Yekutieli to control for false discovery rate (FDR). The results show that the antibodies described herein successfully detected/quantified TDP-43 in human plasma samples, both of diseased subjects and healthy controls.

### Example 8B: Immunoassays for Detection and Quantification of Phospho TDP-43 in Biofluids with ACI-7069-636E5-Ab1, ACI-7071-4665-B5-Ab2, ACI-8071-943.7H9-Ab1, ACI-8072-946.4G5-Ab1 and ACI-7071-809F12-Ab1-rec2

For an assay measuring TDP-43, ACI-7071-809F12-Ab1-rec2 was conjugated to paramagnetic beads serving as a capture reagent and ACI-7071-4665-B5-R3B-Ab2 was biotinylated using EZ-Link™ Sulfo-NHS-LC-Biotin or EZ-Link™ NHS-PEG4-Biotin (both by ThermoFisher Scientific) to serve as a detector antibody. For an assay targeting pS403/pS404 TDP-43, ACI-7069-636E5-Ab1 or ACI-7071-809F12-Ab1-rec2 was conjugated to paramagnetic beads serving as a capture reagent and ACI-8071-943.7H9-Ab1 was biotinylated using EZ-Link™ Sulfo-NHS-LC-Biotin or EZ-Link™ NHS-PEG4-Biotin (both by ThermoFisher Scientific) to serve as a detector antibody. For an assay targeting pS409/pS410 TDP-43, ACI-7071-809F12-Ab1-rec2 was conjugated to paramagnetic beads serving as a capture reagent and ACI-8072-946.4G5-Ab1 was biotinylated using EZ-Link™ Sulfo-NHS-LC-Biotin or EZ-Link™ NHS-PEG4-Biotin (both by ThermoFisher Scientific) to serve as a detector antibody. Assays were performed as described in Example 8A. Following the Quanterix SIMOA® assay definition, for each assay the lower limit of detection (LLOD) was determined as the mean blank for a readout of average enzyme per bead (AEB) plus 2.5-times the standard deviation. The lower limit of quantification (LLOQ) was determined as the lowest concentration of the calibrator with a pooled coefficient of variation (CV)≤20% and a concentration back-calculated from the calibration curve between 80 and 120% of the expected concentration. Assays were repeated on separate occasions to evaluate inter-assay variability.

The characteristic assay quality attributes for selected antibody pairs (for both Example 8A and 8B) measuring TDP-43 or phospho TDP-43 in human biofluids using the Quanterix SIMOA © immunoassay platform are presented in Table 10. Immunoassays were developed with different antibody pairs covering different epitope regions, allowing to quantify TDP-43 and phospho-TDP-43 with a femtomolar lower limit of quantification.

TABLE 10
TDP-43 and phospho TDP-43 biofluid assays setup and developed
on the Quanterix SIMOA ® immunoassay platform.

| | | TDP-43 | | TDP-43 | | |
|---|---|---|---|---|---|---|
| TDP-43 assay | Capture | capture | Detect | detect | LLOD | LLOQ |
| targets | antibody | epitope | antibody | epitope | (pM) | (pM) |
| RRM2/ | ACI-7071-809F12-Ab1-rec2 | 199-213 | ACI-7071-4665-B5-R3B-Ab2 | 366-369 | 0.02 | 0.09 |
| C-term | | | | | | |
| C-term/ | ACI-7069-636E5-Ab1 | 358-361 | ACI-8071-943.7H9-Ab1 | pS403/pS404 | 0.13 | 0.50 |
| pS403/pS404 | | | | | | |
| RRM2/ | ACI-7071-809F12-Ab1-rec2 | 199-213 | ACI-8071-943.7H9-Ab1 | pS403/pS404 | 0.17 | 0.61 |
| pS403/pS404 | | | | | | |
| RRM2/ | ACI-7071-809F12-Ab1-rec2 | 199-213 | ACI-8072-946.4G5-Ab1 | pS409/pS410 | 0.02 | 0.80 |
| pS409/pS410 | | | | | | |

TDP-43 RNA recognition motif 2 (RRM2). TDP-43 carboxy-terminus (C-term). Lower limit of detection (LLOD). Lower limit of quantification (LLOQ).

### Example 9: Detection and Quantification of TDP-43 in Platelets

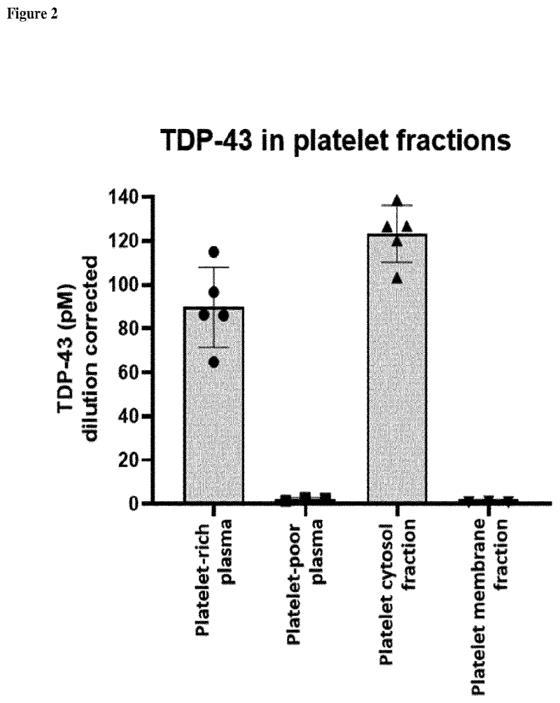

The TDP-43 detection assay of Example 8A (pairing antibodies ACI-7071-4665-B5-R3B-Ab2 as detect antibody and ACI-7071-809F12-Ab1-rec2 as capture antibody) was used to measure TDP-43 in blood compartments from healthy control subjects by isolating platelet fractions using differential centrifugation and measuring TDP-43 in platelet-rich plasma, platelet-poor plasma, and in cytosolic and membrane enriched platelet fractions. Briefly, fresh blood from healthy subjects was collected, and within 2 hr from collection, was centrifuged at 200×g for 10 min at 22° C. Taking care not to disturb the buffy coat, an aliquot of supernatant was collected as platelet-rich plasma and transferred into vials for storage at −80° C. until use. The remaining supernatant was centrifuged at 5′000×g for 20 min at 22° C. to separate platelet-poor plasma from the platelet pellet. Supernatant was carefully collected and transferred into vials for storage at −80° C. until use, and the pellet was gently washed in freshly prepared EDTA in Earle's Balanced Salt Solution (EBSS). To separate platelet cytosol from the membrane fraction, the pellet was lysed in 1×RIPA buffer (Pierce), containing protease (cOmplete, Roche) and phosphatase (PhosSTOP, Roche) inhibitor cocktails using the same volume as initially used to separate platelet-poor from platelet-rich plasma. Samples were sonicated five times for 10 seconds on ice using a probe sonicator at 30% amplitude, and then centrifuged 20′000×g for 10 min at 4° C. to separate the platelet cytosol from the membrane fraction. The supernatant was stored at −80° C. until use, and the pellet resuspended in assay diluent and stored at −80° C. until use. Human TDP-43 was measured in each fraction using a SIMOA® assay as described in Example 8A. Quantifications for TDP-43 were adjusted for the dilution differences of each fraction. Results demonstrated that TDP-43 in blood is almost entirely represented by TDP-43 in platelets, primarily in the platelet cytosol fraction (FIG. 2). Data are represented as means±SD. The results show that the antibodies described herein successfully detected/quantified TDP-43 in human platelet samples and are well-suited for the detection and measurements of TDP-43 in high-sensitivity biofluid immunoassays.

These examples show that the characteristics of these TDP-43 binding molecules (antibodies) are well-suited for their intended uses such as the detection and measurements of pathological TDP-43 post-translational species, particularly known to be implicated in TDP-43 proteinopathies.

These characteristics include phospho-site specificity, high affinity, binding to human TDP-43 pathology in brain tissues, and utility as part of high-sensitivity biofluid immunoassays.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

1.-74. (canceled)

75. A TDP-43 binding molecule, preferably which binds phosphorylated TDP-43, which comprises:
a. a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
b. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
d. a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
e. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
f. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
g. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, comprising a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
h. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
i. a VH-CDR1 comprising the amino acid sequence of SEQ ID NO: 111, a VH-CDR2 comprising the amino acid sequence of SEQ ID NO: 112, a VH-CDR3 comprising the amino acid sequence of SEQ ID NO: 113, a VL-CDR1 comprising the amino acid sequence of SEQ ID NO: 115, a VL-CDR2 comprising the amino acid sequence of SEQ ID NO: 116 and a VL-CDR3 comprising the amino acid sequence of SEQ ID NO: 117.

76. The TDP-43 binding molecule of claim 75, which specifically binds phosphorylated TDP-43, wherein the binding molecule comprises:
a) a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
b) a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
c) a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16 and a VL-CDR3 comprising the amino acid sequence of SEQ ID NO: 37; or
d) a VH-CDR1 comprising the amino acid sequence of SEQ ID NO: 41, a VH-CDR2 comprising the amino acid sequence of SEQ ID NO: 42, a VH-CDR3 comprising the amino acid sequence of SEQ ID NO: 33, a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO:47; or
e) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57; or
f) a VH-CDR1 comprising the amino acid sequence of SEQ ID NO: 61, a VH-CDR2 comprising the amino acid sequence of SEQ ID NO: 62, VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
g) a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 75, a VL-CDR2 comprising the amino acid sequence of SEQ ID NO: 76 and a VL-CDR3 comprising the amino acid sequence of SEQ ID NO: 77; or
h) a VH-CDR1 comprising the amino acid sequence of SEQ ID NO: 111, a VH-CDR2 comprising the amino acid sequence of SEQ ID NO: 112, a VH-CDR3 comprising the amino acid sequence of SEQ ID NO: 113, a VL-CDR1 comprising the amino acid sequence of SEQ ID NO: 115, a VL-CDR2 comprising the amino acid sequence of SEQ ID NO: 116 and a VL-CDR3 comprising the amino acid sequence of SEQ ID NO: 117.

77. The TDP-43 binding molecule of claim 76, wherein the binding molecule according to any one of (a), (c), (f) or (g) binds TDP-43 positive inclusions, optionally wherein binding to TDP-43 positive inclusions is determined by immunohistochemistry.

78. The TDP-43 binding molecule of claim 75, which binds to an epitope comprising:
a. phosphorylated amino acid residues pS375, pS379, pS403, pS404, pS409 and/or pS410 of human TDP-43 (SEQ ID NO: 1) or to an equivalent epitope in non-human TDP-43;
b. phosphorylated amino acid residues pS403 and/or pS404 of human TDP-43 (SEQ ID NO: 1) or to an equivalent epitope in non-human TDP-43, optionally wherein the TDP-43 binding molecule binds to the epitope with a KD of 13 nM or less, preferably 5 nM or less, preferably 2 nM or less, preferably wherein the epitope comprises or consists of GFNGGFG(pS)(pS)MDSKS (SEQ ID NO: 8) corresponding to amino acid positions 396 to 409 of SEQ ID NO: 1, more preferably wherein the KD is measured by surface plasmon resonance;
c. phosphorylated amino acid residues pS409 and/or pS410 of human TDP-43 (SEQ ID NO: 1) or to an equivalent epitope in non-human TDP-43, optionally wherein the TDP-43 binding molecule binds the epitope with a KD of 3.5 nM or less, preferably 2.5 nM or less, preferably 1.7 nM or less, preferably wherein the epitope comprises or consists of FGSSMDSK(pS)(pS)GWG (SEQ ID NO: 9) corresponding to amino acid positions 401 to 413 of SEQ ID NO: 1, more preferably wherein the KD is measured by surface plasmon resonance; or
d. phosphorylated amino acid residues pS375 and/or pS379 of human TDP-43 (SEQ ID NO: 1) or to an equivalent epitope in non-human TDP-43, optionally wherein the TDP-43 binding molecule binds the epitope with a KD of 21.5 nM or less, preferably wherein the epitope comprises or consists of GNNSY(pS)GSN(pS)GAAIG (SEQ ID NO: 5) corresponding to amino acid positions 370 to 384 of SEQ ID NO: 1, more preferably wherein the KD is measured by surface plasmon resonance.

79. The TDP-43 binding molecule of claim 75 which binds TDP-43 positive inclusions, wherein the binding molecule comprises:
a. a VH-CDR1 comprising the amino acid sequence of SEQ ID NO: 11, a VH-CDR2 comprising the amino acid sequence of SEQ ID NO: 12, a VH-CDR3 comprising the amino acid sequence of SEQ ID NO: 13, a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17; or
b. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino acid sequence of SEQ ID NO: 35, a VL-CDR2 comprising the amino acid sequence of SEQ ID NO: 16; and a VL-CDR3 comprising the amino acid sequence of SEQ ID NO: 37; or
c. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26, and a VL-CDR3 comprising the amino acid sequence of SEQ ID NO: 57; or
d. a VH-CDR1 comprising the amino acid sequence of SEQ ID NO: 51, a VH-CDR2 comprising the amino acid sequence of SEQ ID NO: 52, a VH-CDR3 comprising the amino acid sequence of SEQ ID NO: 53, a VL-CDR1 comprising the amino acid sequence of SEQ ID NO: 75, a VL-CDR2 comprising the amino acid sequence of SEQ ID NO: 76, and a VL-CDR3 comprising the amino acid sequence of SEQ ID NO: 77; or
e. a VH-CDR1 comprising the amino acid sequence of SEQ ID NO: 81, a VH-CDR2 comprising the amino acid sequence of SEQ ID NO: 82, VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16, and a VL-CDR3 comprising the amino acid sequence of SEQ ID NO: 87.

80. The TDP-43 binding molecule of claim 75 which binds misfolded aggregated TDP-43 and non-aggregated physiological TDP-43, wherein the TDP-43 binding molecule comprises a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87.

81. The TDP-43 binding molecule of claim 80, which binds to an epitope within amino acids residues 361-414 of human TDP-43 (SEQ ID NO: 1) or to an equivalent epitope in non-human TDP-43, optionally wherein the TDP-43 binding molecule binds human TDP-43 (SEQ ID NO: 1) with a KD of 0.39 nM or less, preferably wherein the KD is measured by surface plasmon resonance.

82. The TDP-43 binding molecule of claim 75 which comprises:
a. a Heavy Chain Variable Region (VH) comprising the sequence of SEQ ID NO: 10 or a Heavy Chain Variable Region (VH) having at least 96%, 97%, 98% or 99% sequence identity to the amino acid sequence of SEQ ID NO: 10; and a Light Chain Variable Region (VL) comprising the sequence of SEQ ID NO: 14 or a Light Chain Variable Region (VL) having at least 95%, 96%, 97%, 98% or 99% sequence identity to the amino acid sequence of SEQ ID NO: 14; or
b. a Heavy Chain Variable Region (VH) comprising the sequence of SEQ ID NO: 20 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 20; and
a Light Chain Variable Region (VL) comprising the sequence of SEQ ID NO: 24 or a Light Chain Variable Region (VL) having at least 96%, 97%, 98% or 99% sequence identity to the amino acid sequence of SEQ ID NO: 24; or
c. a Heavy Chain Variable Region (VH) comprising the sequence of SEQ ID NO: 30 or a Heavy Chain Variable Region (VH) having at least 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Heavy Chain Variable Region (VH) comprising the sequence of SEQ ID NO: 40 or a Heavy Chain Variable Region (VH) having at 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 94%, 95%, 96%, 97%, 98% or 99% sequence identity to the amino acid sequence of SEQ ID NO: 44; or
e. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 54; or
f. a Heavy Chain Variable Region (VH) comprising the sequence of SEQ ID NO: 60 or a Heavy Chain Variable Region (VH) having at least 98% or 99% sequence identity to the amino acid sequence of SEQ ID NO: 60; and a Light Chain Variable Region (VL) comprising the sequence of SEQ ID NO: 64; or
g. a Heavy Chain Variable Region (VH) comprising the sequence of SEQ ID NO: 50 or a Heavy Chain Variable Region (VH) having at least 93%, 94%, 95%, 96%, 97%, 98% or 99% sequence identity to the amino acid sequence of SEQ ID NO: 50; and a Light Chain Variable Region (VL) comprising the sequence of SEQ ID NO: 74; or
h. a Heavy Chain Variable Region (VH) comprising the sequence of SEQ ID NO: 80 or a Heavy Chain Variable Region (VH) having at least 94%, 95%, 96%, 97%, 98%, or 99% sequence identity to the amino acid sequence of SEQ ID NO: 80; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
i. a Heavy Chain Variable Region (VH) comprising the sequence of SEQ ID NO: 110 or a Heavy Chain Variable Region (VH) having at least 92%, 93%, 94%, 95%, 96%, 97%, 98%, or 99% sequence identity to the amino acid sequence of SEQ ID NO: 110; and a Light Chain Variable Region (VL) comprising the sequence of SEQ ID NO: 114.

83. The TDP-43 binding molecule of claim 75, which is a) an antibody or an antigen-binding fragment thereof, or b) an IgA, IgD, IgE, IgM, IgG1, IgG2, IgG2a, IgG2b, IgG3 or IgG4 antibody or antigen-binding fragment thereof.

84. The TDP-43 binding molecule of claim 75, wherein the binding molecule is an immunoconjugate, optionally wherein the immunoconjugate comprises:
a. paramagnetic beads;
b. biotin; or
c. an additional therapeutic molecule.

85. The TDP-43 binding molecule of claim 75 for research use, in particular as an analytical tool or a reference molecule.

86. A method for preventing, alleviating or treating a TDP-43 proteinopathy in a subject in need thereof, the method comprising administering an effective amount of the TDP-43 binding molecule of claim 75 to the subject.

87. A method for diagnosing a TDP-43 proteinopathy in a subject in need thereof, the method comprising administering an effective amount of the TDP-43 binding molecule of claim 75 to the subject.

88. The method according to claim 86, wherein the TDP-43 proteinopathy is either:
a. a disease, disorder and/or abnormality associated with TDP-43 aggregates selected from the group consisting of Amyotrophic Lateral Sclerosis (ALS), Frontotemporal Dementias (FTDs, including Argyrophilic grain disease), Frontotemporal Lobar Degeneration with Motor Neuron Disease FTLD-MND (also known as ALS-FTD), Behavioural Variant Frontotemporal Dementia (bvFTD), Semantic Variant Primary Progressive Aphasia (svPPA), Nonfluent/Agrammatic Primary Progressive Aphasia (naPPA), Alzheimer's Disease (AD), Down Syndrome (DS), familial British dementia, Parkinson's Disease (PD) and related disorders (including PD with Dementia (PDD), dementia with Lewy Bodies (DLB), multiple system atrophy (MSA)), Corticobasal degeneration (CBD), Niemann-Pick disease (NP, including NP type C), Facial-Onset Sensory Motor Neuronopathy (FOSMN), limbic-predominant age-related TDP-43 encephalopathy (LATE), Chronic Traumatic Encephalopathy, Perry syndrome, Paget disease, polyglutamine diseases (such as Huntington's disease (HD) and spinocerebellar ataxia type 3 (SCA3, also known as Machado Joseph disease)), hippocampal sclerosis with dementia, myofibrillar myopathies (e.g. inclusion body myositis, inclusion body myopathy, oculopharyngeal muscular dystrophy with rimmed vacuoles), Primary Lateral Sclerosis (PLS), Progressive Muscular Atrophy; or
b. a disease arising from mutations or variant-associated risk alleles of the progranulin (GRN), TARDBP, C9ORF72, valosin-containing protein (VCP), angiogenin (ANG), desmin (DES), myotilin (MYOT), TMEM106B, huntingtin (HTT), ataxin 3 (ATXN3) genes.

89. The method according to claim 88, wherein the disease, disorder and/or abnormality associated with TDP-43, or TDP-43 proteinopathy, is:
a. amyotrophic lateral sclerosis (ALS);
b. Alzheimer's disease (AD);
c. Frontotemporal dementia (FTD);
d. limbic-predominant age-related TDP-43 encephalopathy (LATE); or
e. Frontotemporal Lobar Degeneration with Motor Neuron Disease FTLD-MND.

90. A pharmaceutical composition comprising the TDP-43 binding molecule of claim 75 and a pharmaceutically acceptable carrier or excipient.

91. A diagnostic composition comprising the TDP-43 binding molecule of claim 75 and an acceptable carrier or excipient.

92. A nucleic acid molecule encoding the TDP-43 binding molecule of claim 75, optionally comprising the nucleotide sequence set forth as SEQ ID NO: 18, SEQ ID NO: 19, SEQ ID NO: 28, SEQ ID NO: 29, SEQ ID NO: 38, SEQ ID NO: 39, SEQ ID NO: 48, SEQ ID NO: 49, SEQ ID NO: 58, SEQ ID NO: 59, SEQ ID NO: 68, SEQ ID NO: 69, SEQ ID NO: 79, SEQ ID NO: 88, SEQ ID NO: 89, SEQ ID NO: 118 or SEQ ID NO: 119.

93. A recombinant vector or expression vector comprising the nucleic acid of claim 92.

94. A host cell comprising the nucleic acid of claim 92, or a host cell comprising a recombinant vector or expression vector comprising the nucleic acid, optionally wherein the host cell expresses the TDP-43 binding molecule.

95. A cell-free expression system containing the expression vector of claim 93.

96. A method for producing a TDP-43 binding molecule, in particular an antibody or antigen-binding fragment thereof, comprising the steps of:
a. culturing a host cell or cell-free expression system comprising the expression vector of claim 93 under conditions suitable for producing the binding molecule, in particular the antibody or antigen-binding fragment thereof; and
b. isolating the binding molecule, in particular the antibody or antigen-binding fragment thereof.

97. A method for detection and/or quantification of TDP-43 or phosphorylated TDP-43 in a sample, the method comprising contacting the sample with the TDP-43 binding molecule of claim 75, wherein the sample is saliva, urine, nasal secretion, blood (including whole blood, plasma and serum, platelets rich plasma, platelets cytosol fraction), brain and/or CSF sample, brain and/or ISF sample, more particularly blood, brain, CSF and/or ISF sample.

98. A method of using a TDP-43 binding molecule of claim 75 in a pairing assay comprising the steps of:
a. incubating a sample with a capture antibody and a detect antibody;
b. incubating the mixture obtained in step a with a reagent suitable for detection by the detect antibody;
c. measuring the signal emitted by the detect antibody;
optionally wherein the capture and/or detect antibody is selected from an antibody as defined in claim 75, further optionally wherein the reagent is Streptavidin-β-D-Galactosidase.

99. The method of claim 98, wherein:
a. the capture antibody and/or detect antibody binds phosphorylated TDP-43, preferably specifically binds phosphorylated TDP-43; or
b. the capture antibody and/or detect antibody binds misfolded aggregated TDP-43 and non-aggregated physiological TDP-43.

100. The method of claim 98, wherein the capture antibody comprises:
i. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
ii. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
iii. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.

101. The method of claim 98, wherein the detect antibody comprises:
i.a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87; or
ii. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
iii. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107; or
iv. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27; or
v. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57.

102. The method of claim 98, wherein the detect antibody comprises:
a. a VH-CDR1 comprising the amino acid sequence of SEQ ID NO: 81, a VH-CDR2 comprising the amino acid sequence of SEQ ID NO: 82, a VH-CDR3 comprising the amino acid sequence of SEQ ID NO: 83, a VL-CDR1 comprising the amino acid sequence of SEQ ID NO: 85, a VL-CDR2 comprising the amino acid sequence of SEQ ID NO: 16 and a VL-CDR3 comprising the amino acid sequence of SEQ ID NO: 87;
and the capture antibody comprises:
b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107; or
wherein the detect antibody comprises:
a. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27;
and the capture antibody comprises:
b. a VH-CDR1 comprising the amino acid sequence of SEQ ID NO: 91, a VH-CDR2 comprising the amino acid sequence of SEQ ID NO: 92, a VH-CDR3 comprising the amino acid sequence of SEQ ID NO: 93, a VL-CDR1 comprising the amino acid sequence of SEQ ID NO: 95, a VL-CDR2 comprising the amino acid sequence of SEQ ID NO: 96 and a VL-CDR3 comprising the amino acid sequence of SEQ ID NO: 97; or
wherein the detect antibody comprises:
a. a VH-CDR1 comprising the amino acid sequence of SEQ ID NO: 21, a VH-CDR2 comprising the amino acid sequence of SEQ ID NO: 22, a VH-CDR3 comprising the amino acid sequence of SEQ ID NO: 23, a VL-CDR1 comprising the amino acid sequence of SEQ ID NO: 25, a VL-CDR2 comprising the amino acid sequence of SEQ ID NO: 26 and a VL-CDR3 comprising the amino acid sequence of SEQ ID NO: 27;
and the capture antibody comprises:
b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107; or
wherein the detect antibody comprises:
a. a VH-CDR1 comprising the amino acid sequence of SEQ ID NO: 61, a VH-CDR2 comprising the amino acid sequence of SEQ ID NO: 62, a VH-CDR3 comprising the amino acid sequence of SEQ ID NO: 63, a VL-CDR1 comprising the amino acid sequence of SEQ ID NO: 55, a VL-CDR2 comprising the amino acid sequence of SEQ ID NO: 26 and a VL-CDR3 comprising the amino acid sequence of SEQ ID NO: 57;
and the capture antibody comprises:
b. a VH-CDR1 comprising the amino acid sequence of SEQ ID NO: 101, a VH-CDR2 comprising the amino acid sequence of SEQ ID NO: 102, a VH-CDR3 comprising the amino acid sequence of SEQ ID NO: 103, a VL-CDR1 comprising the amino acid sequence of SEQ ID NO: 105, a VL-CDR2 comprising the amino acid sequence of SEQ ID NO: 26 and a VL-CDR3 comprising the amino acid sequence of SEQ ID NO: 107.

103. A method of quantifying phosphorylated TDP-43 in a sample, the method comprising contacting the sample with a TDP-43 binding molecule according to claim 75, optionally wherein the sample was obtained from a subject and wherein the sample is human blood, cerebrospinal fluid (CSF), interstitial fluid (ISF), saliva, nasal secretion and/or urine, preferably blood, CSF or ISF, optionally wherein the method comprises comparing detected TDP-43 levels from the sample to a control, optionally wherein the control comprises phosphorylated TDP-43, further optionally wherein the control was determined using a known amount of calibrator for phosphorylated TDP-43.

104. A method for monitoring a disease, disorder and/or condition associated with TDP-43 at two or more time points using samples from a subject comprising contacting the samples with a TDP-43 binding molecule of claim 75, wherein;
a. a change of levels of TDP-43 and/or phosphorylated TDP-43 in the later sample compared with one or more earlier samples is indicative of modification of a disease, disorder and/or condition associated with TDP-43; or
b. no significant change of levels of TDP-43 and/or phosphorylated TDP-43 in the later sample compared with one or more earlier samples are indicative of lack of modification of a disease, disorder and/or condition associated with TDP-43; or
c. higher levels of TDP-43 and/or phosphorylated TDP-43 in the later sample compared with one or more earlier samples are indicative of progression of a disease, disorder and/or condition associated with TDP-43; or
d. lower levels of TDP-43 and/or phosphorylated TDP-43 in the later sample compared with one or more earlier samples are indicative of regression of a disease, disorder and/or condition associated with TDP-43; or
e. no significant change of levels of TDP-43 and/or phosphorylated TDP-43 in the later sample compared with one or more earlier samples are indicative of lack of progression of a disease, disorder and/or condition associated with TDP-43; or
f. higher levels of TDP-43 and/or phosphorylated TDP-43 in the later sample compared with one or more earlier samples are indicative of regression of a disease, disorder and/or condition associated with TDP-43; or
g. lower levels of TDP-43 and/or phosphorylated TDP-43 in the later sample compared with one or more earlier samples are indicative of progression of a disease, disorder and/or condition associated with TDP-43; or
optionally wherein the method performed at multiple time points in matched samples between treatment and placebo groups in order to monitor the effectiveness of a candidate therapy over a defined time period.

105. A kit for diagnosis of a disease, disorder and/or abnormality associated with TDP-43, or a TDP-43 proteinopathy, or for use in a method of quantifying phosphorylated TDP-43 in a sample, comprising a TDP-43 binding molecule according to claim 75, optionally wherein the kit of claim comprises a TDP-43 binding molecule according to claim 75 as capture antibody and a different TDP-43 binding molecule according to claim 75 as detect antibody, optionally wherein the kit further comprises magnetic particles to which the capture antibody is, or can be, attached, further optionally wherein the detect antibody is labelled, either directly or indirectly, optionally wherein the kit further comprises a container that contains the TDP-43 binding molecule(s).
